(19) United States
(12) Patent Application Publication
(10) Pub. No.: US 2013/0017151 A1
(43) Pub. Date: Jan. 17, 2013

(54) SUBSETS OF ANTIGEN-PRESENTING CELLS (APCs) IN THE HUMAN VAGINA AND THEIR DISTINCT FUNCTIONS

(71) Applicants: SangKon Oh, Baltimore, MD (US); Dorothee Duluc, Dallas, TX (US); Julien Gannevat, Dallas, TX (US)

(72) Inventors: SangKon Oh, Baltimore, MD (US); Dorothee Duluc, Dallas, TX (US); Julien Gannevat, Dallas, TX (US)

(73) Assignee: BAYLOR RESEARCH INSTITUTE, Dallas, TX (US)

(21) Appl. No.: 13/546,190

(22) Filed: Jul. 11, 2012

Related U.S. Application Data
(60) Provisional application No. 61/506,496, filed on Jul. 11, 2011.

(51) Int. Cl. C12N 5/0784; C12N 5/0786; A61K 49/00; A61P 15/02; A61K 39/395; A61K 35/12

(52) U.S. Cl. 424/9.2; 424/172.1; 424/133.1; 424/178.1; 424/93.7; 435/325; 435/372; 435/375

(57) ABSTRACT

Compositions and methods for generating dendritic cell (DC)-targeting vaccines against vaginal infections, including but not limited to sexually transmitted diseases, are disclosed herein. The present invention reports the isolation of at least four major subsets of myeloid-originated antigen-presenting cells (APCs) that possess distinct phenotypes and functions in directing immune responses, namely, Langerhans cells (LCs: E-cadherin⁺CD207⁺CD205⁺), CD1c⁺CD14⁻ DCs (DC-ASGPR⁺CD209^{+/-}Dectin-1^{+/-}), and CD1c⁺CD14⁺ DCs (CD209^{+/-}DC-ASGPR^{+/-}) all expressing high levels of CD11c, CD83, and CCR6, and are more potent than CD1c⁻CD14⁺ macrophages (CD163⁺CD209⁺DC-ASGPR^{+/-}Dectin-1^{+/-}LOX-1⁺CD1d⁺) at eliciting naïve T cell proliferation The compositions, methods and vaccines of the present invention are directed towards the four functionally distinct major subsets of antigen-presenting cells (APCs) that can differentially contribute to the host immune response in the female genital tract, including the vagina.

### CROSS-REFERENCE TO RELATED APPLICATIONS

This application claims priority to U.S. Provisional Application Ser. No. 61/506,496 filed Jul. 11, 2011, the entire contents of which are incorporated herein by reference.

### STATEMENT OF FEDERALLY FUNDED RESEARCH

This invention was made with U.S. Government support under Contract No. 1RC1AI087379-01 awarded by the National Institutes of Health (NIH). The government has certain rights in this invention.

### TECHNICAL FIELD OF THE INVENTION

The present invention relates in general to immunology, dendritic cell (DC)-targeting vaccines and therapeutics and more particularly to the discovery of four functionally distinct major subsets of antigen-presenting cells (APCs) the human vaginal mucosa that can differentially contribute to the host immune response in the female genital tract, including the vagina.

### REFERENCE TO A SEQUENCE LISTING

None.

### BACKGROUND OF THE INVENTION

Without limiting the scope of the invention, its background is described in connection with compositions, methods, and studies related to the immunological profile of the vagina.

European Patent Application No. EP0779817 issued to Clancy (1997) discloses an enteral non-adjuvanated vaccine comprising a killed microorganism, which infects the vagina. The microorganism may be a fungus such as Candida albicans, a bacterium such as Gardnerella vaginalis or Neisseria gonorrhea, a protozoan such as Trichomonas vaginalis, a virus such as herpes genitalis. The absence of the adjuvant gives a significant improvement in clearance of the microorganisms, compared to the adjuvanated compositions.

Iijima et al. (2008) have provided a comprehensive review of DCs and their function in the genitourinary tract in females. According to the Iijima paper, the genitourinary tract is constantly exposed to numerous agents of sexually transmitted infections (STIs). To combat these STIs, several subsets of DCs and macrophages are strategically localized within the GU tract. In the female genital mucosa, recruitment and function of these APCs are uniquely governed by sex hormones. The paper also discusses the divergent roles of these cells in immune defense against STIs as well as in maternal tolerance to the fetus.

The immune events in the vagina of mice intravaginally infected with highly virulent herpes simplex virus type 2 (HSV-2) strain 186 were studied by Ohara et al. (2000). Ohara and co-workers compared HSV-2 strain 186 with those induced by HSV type 1 strain KOS, a widely known laboratory strain. Although there was no significant difference between 186 and KOS in the viral replication in the initial stage of infection, inadequate and delayed clearance of virus from the vaginal mucosa was observed in 186-challenged mice. The induction of antigen-presenting cells (APC) such as dendritic cells (DC) and macrophages (MØ) in the vagina was slow in 186-challenged mice, and the number of T cells in the vagina in 186-challenged mice was much lower than that in KOS-challenged mice. Furthermore, the level of IL-12 as well as that of IFN-gamma was significantly lower in 186-challenged mice than in KOS-challenged mice, while the level of IL-4 in 186-challenged mice was higher than that in KOS-challenged mice. The Ohara paper suggests that the weak activation of epithelial cells and the delayed induction of APC by 186-infection may be involved in the inadequate activation of T cells and the ineffective virus clearance from the vaginal mucosa. Miller et al. (1992) studied the morphology of the mucosa-associated lymphoid tissue in the genital tract of rhesus monkeys. The findings of the Miller study indicated that CD1a⁺ Langerhans cells were present in the stratified squamous epithelium of the vagina (14 animals) and ectocervix (11 animals). Surprisingly, CD1a⁺ dendritic cells were also found within the columnar epithelium of the endocervix (5 animals). Moderate numbers of CD68⁺ macrophages were located in the submucosa of the vagina, ectocervix, and endocervix of all the monkeys. In all of the animals, moderate numbers of CD8⁺ lymphocytes were present in the submucosa and squamous epithelia of the vagina and ectocervix. Variable numbers of CD20⁺ B cells and CD4⁺ lymphocytes were located in the submucosa of all the areas examined.

Finally, Seavey and Mosmann (2009) have explored the use of vaginal immunization as a strategy to induce mucosal immunity in the female reproductive tract. Seavey and Mosmann suggest that female reproductive tract displays unique immunological features that has probably evolved to inhibit anti-paternal T cell responses after insemination to allow successful pregnancy. The authors confirmed their hypothesis by using estradiol that prevented antigen loading of vaginal APCs after vaginal immunization.

### SUMMARY OF THE INVENTION

Compositions and methods for prophylaxis, treatment, or amelioration of symptoms of vaginal infections, including but not limited to sexually transmitted diseases are disclosed in various embodiments herein. The compositions and methods disclosed herein include the isolation, and targeting, of four major subsets of myeloid-originated antigen-presenting cells (APCs) that possess distinct phenotypes and functions in directing immune responses, namely, Langerhans cells (LCs: E-cadherin⁺CD207⁺CD205⁺), CD1c⁺CD14⁻ DCs (DC⁻ASGPR⁺CD209^{+/-}Dectin-1^{+/-}), and CD1c⁺CD14⁺DCs (CD209^{+/-}DC⁻ASGPR^{+/-}) all expressing high levels of CD11c, CD83, and CCR6, and are more potent than CD1c⁻CD14⁺ macrophages (CD163⁺CD209⁺DC⁻ASGPR^{+/-}Dectin-1^{+/-}LOX-1⁺CD1d⁺) at eliciting naïve T cell proliferation.

An isolated immunogenic composition is disclosed herein, that comprises at least one subset of antigen presenting cells (APCs), wherein the APCs possess a distinct phenotype and the subset is selected from at least one of Langerhans cells (LCs) E-cadherin⁺CD207⁺CD205⁺, vaginal CD1c⁺CD14⁻DC-ASGPR⁺CD209^{+/-}Dectin-1^{+/-} dendritic cells (DCs), vaginal CD1c⁺CD14⁺CD209^{+/-}DC-ASGPR^{+/-}DCs, CD1c⁻CD14⁺ CD163⁺CD209⁺DC-ASGPR^{+/-}Dectin-1^{+/-}LOX-1⁺CD1d⁺ macrophages, or CD1c⁻CD14⁻ DCs.

The APCs disclosed hereinabove are myeloid-originated APCs and are isolated from a vaginal tissue or a vaginal mucosa from a human or animal subject. In one aspect the immunogenic composition disclosed hereinabove induces proliferation of one or more T cells towards a Th-1 type, or a Th2-type, or both. The T cell proliferation by the immunogenic composition disclosed hereinabove results in an induction in expression of mucosal homing receptors, CD103, β7 integrin, CCR4, CXCR3, or any combinations thereof by the T cells.

Another embodiment disclosed herein relates to a composition comprising at least one antigen presenting cell (APC) subset, wherein the composition comprises: (i) one or more vaginal Langerhans cells (LCs) E-cadherin⁺CD207⁺CD205⁺, wherein the LCs express one or more surface molecules selected from the group consisting of CD1a, E-cadherin, or both, CD86, CD83 or any combinations thereof, (ii) one or more vaginal CD1c⁺CD14⁻ DC-ASGPR⁺CD209^{+/-}Dectin-1^{+/-} dendritic cells (DCs), wherein the CD1c⁺CD14⁻ DCs express CD86, CD83, or both, (iii) one or more vaginal CD1c⁺CD14⁺ CD209^{+/-}DC-ASGPR^{+/-} DCs, wherein the CD1c⁺CD14⁺ DCs express CD86, CD83, or both and (iv) one or more vaginal CD1c⁻CD14⁺CD163⁺CD209⁺DC-ASGPR^{+/-}Dectin-1^{+/-}LOX-1⁺CD1d⁺ macrophages, wherein the CD1c⁻CD14⁺ macrophages express CD86, CD163, or both.

In yet another embodiment the instant invention discloses an immunogenic composition comprising a combination of antigen presenting cell (APC) subsets isolated from a vaginal tissue or mucosa from a human or animal subject comprising: one or more vaginal Langerhans cells (LCs) E-cadherin⁺CD207⁺CD205⁺, wherein the LCs express one or more surface molecules selected from the group consisting of CD1a, E-cadherin, or both, CD86, CD83 or any combinations thereof, one or more vaginal CD1c⁺CD14⁻ DC-ASGPR⁺CD209^{+/-}Dectin-1^{+/-} dendritic cells (DCs), wherein the CD1c⁺CD14⁻ DCs express CD86, CD83, or both, one or more vaginal CD1c⁺CD14⁺ CD209^{+/-}DC-ASGPR^{+/-} DCs, wherein the CD1c⁺CD14⁺ DCs express CD86, CD83, or both, and one or more vaginal CD1c⁻CD14⁺CD163⁺CD209⁺DC-ASGPR^{+/-}Dectin-1^{+/-LOX-}1⁺CD1d⁺ macrophages, wherein the CD1c⁻CD14⁺ macrophages express CD86, CD163, or both.

In one embodiment the instant invention describes an immunostimulatory composition for generating a vaginal immune response, for a prophylaxis, a therapy or any combination thereof in a human or animal subject comprising: one or more vaginal anti-dendritic cell (DC)-specific antibodies or fragments thereof directed towards one or more specific vaginal DC subsets/macrophages loaded or chemically coupled with one or more antigenic peptides, wherein the antigenic peptides are representative of one or more epitopes of the one or more antigens implicated or involved in a disease or a condition against which the immune response, the prophylaxis, the therapy, or any combination thereof is desired, wherein the antibodies or fragments are directed towards one or more antigens selected from the group consisting of E-cadherin⁺, CD207⁺, CD205⁺ CD1c⁺, CD14⁻, DC-ASGPR^{+/-}, CD209^{+/-}, Dectin-1^{+/-}, CD86, CD83, CD209^{+/-}, CD1c⁻, CD14⁺, CD163⁺, LOX-1, CD1d⁺, CD1c⁻, CD14, CD103, β7 integrin, CCR4, CXCR3, and any combinations thereof and a pharmaceutically acceptable carrier, wherein the composition is effective to produce the vaginal immune response, for prophylaxis, for therapy or any combination thereof in the human or animal subject in need of vaginal immunostimulation.

In one aspect of the composition hereinabove the DC subsets/macrophages are selected from the group consisting of Langerhans cells (LCs) E-cadherin⁺CD207⁺CD205⁺, vaginal CD1c⁺CD14⁻DC-ASGPR⁺CD209^{+/-}Dectin-1^{+/-} DCs, vaginal CD1c⁺CD14⁺ CD209^{+/-}DC-ASGPR^{+/-} DCs, CD1c⁻CD14⁺ CD163⁺CD209⁺DC-ASGPR^{+/-}Dectin-1⁺LOX-1⁺CD1d⁺ macrophages, CD1c⁻CD14⁻ DCs, and any combinations thereof. In yet another aspect the DC subsets are present in a vaginal tissue or a vaginal mucosa in the human or animal subject.

In a related aspect the antigenic peptides comprise human immunodeficiency virus (HIV) antigens and gene products selected from the group consisting of gag, pol, and env genes, the Nef protein, reverse transcriptase, string of HIV peptides (Hipo5), a HIVgag-derived p24-PLA HIV gag p24 (gag), and other HIV components, cytomegaloviral antigens, herpes simplex viral antigens, human papilloma virus (HPV) E6 and E7 antigens, antigens from bacteria and fungi selected from the group consisting of Prevotella bivia, Prevotella melaminogenica, Gardnerella vaginalis, Trichomonas vaginalis, Mycoplasma hominis, Mobiluncus species, Neisseria gonorrhoeae, Chlamydia trachomatis, Ureaplasma urealyticus, Candida species, Streptococcus species, and Enterobacteriaceae, or combinations and modifications thereof. In a specific aspect the antigenic peptides are cancer peptides are selected from tumor associated antigens comprising antigens from genitourinary tumors such cervix, uterus, ovarian cancer, vaginal cancer, head and neck cancers caused by HPV infections, or combinations and modifications thereof.

In one aspect the anti-DC-specific antibody is humanized. In another aspect the composition is adapted for intravaginal administration to induce a proliferation of one or more T cells towards Th-1 type, Th2-type, or both. The T cell proliferation described herein results in an induction in expression of mucosal homing receptors, CD103, β7 integrin, CCR4, CXCR3, or any combinations thereof by the T cells.

Another embodiment of the instant invention discloses an immunostimulatory composition for generating a vaginal immune response, for a prophylaxis, a therapy or any combination thereof in a human or animal subject comprising: (i) a vaginal anti-dendritic cell (DC)-specific antibody or fragment thereof with two or more antigen binding sites directed towards one or more specific vaginal DC subsets/macrophages loaded or chemically coupled with one or more antigenic peptides, wherein the antigenic peptides are representative of one or more epitopes of the one or more antigens implicated or involved in a disease or a condition against which the vaginal immune response, the prophylaxis, the therapy, or any combination thereof is desired, wherein the antibody or fragment is directed towards one or more antigens selected from the group consisting of E-cadherin⁺, CD207⁺, CD205⁺CD1c⁺, CD14⁻, DC-ASGPR^{+/-}, CD209^{+/-}, Dectin-1^{+/-, CD}86, CD83, CD209^{+/-}, CD1c⁻, CD14⁺, CD163⁺, LOX-1, CD1d⁺, CD1c⁻, CD14⁻, CD103, β7 integrin, CCR4, CXCR3, and any combinations thereof and (ii) a pharmaceutically acceptable carrier, wherein the composition is effective to produce the vaginal immune response, for prophylaxis, for therapy or any combination thereof in the human or animal subject in need of vaginal immunostimulation. In one aspect the antibody or the fragment has 2, 3, 4, 5, 6, 7, 8, 9, 10, or more antigen binding sites. In another aspect the anti-DC-specific antibody is humanized. In yet another aspect the composition is adapted for intravaginal administration.

One embodiment of the present invention relates to a vaccine composition comprising: (i) one or more vaginal anti-dendritic cell (DC)-specific antibodies or fragments thereof directed towards one or more specific vaginal DC subsets/macrophages loaded or chemically coupled with one or more antigenic peptides, wherein the antigenic peptides are representative of one or more epitopes of the one or more antigens implicated or involved in a disease or a condition against which the immune response, the prophylaxis, the therapy, or any combination thereof is desired, wherein the antibodies or fragments are directed towards one or more antigens selected from the group consisting of E-cadherin⁺, CD207⁺, CD205⁺ CD1c⁺, CD14⁻, DC-ASGPR^{+/-}, CD209^{+/-}, Dectin-1^{+/-}, CD86, CD83, CD209^{+/-}, CD1c⁻, CD14⁺, CD163⁺, LOX-1, CD1d⁺, CD1c⁻, CD14⁻, CD103, β7 integrin, CCR4, CXCR3, and any combinations thereof and (ii) one or more optional pharmaceutically acceptable carriers and adjuvants, wherein the composition is effective to produce an immune response, for a prophylaxis, a therapy or any combination thereof in a human or animal vagina.

In one aspect of the vaccine composition disclosed above the DC subsets/macrophages are selected from the group consisting of Langerhans cells (LCs): E-cadherin⁺CD207⁺CD205⁺, vaginal CD1c⁺CD14⁻ DC-ASGPR⁺CD209^{+/-}Dectin-1^{+/-} DCs, vaginal CD1c⁺CD14⁺ CD209^{+/-}DC-ASGPR^{+/-} DCs, CD1c⁻CD14⁺CD163⁺CD209⁺DC-ASGPR^{+/-}Dectin-1^{+/-}LOX-1⁺CD1d⁺ macrophages, CD1c⁻ CD14⁻ DCs, and any combinations thereof. In another aspect the composition comprises one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines, or combinations and modifications thereof. In yet another aspect the DC subsets are present in a vaginal tissue or a vaginal mucosa in the human or animal subject. In a related aspect the antigenic peptides comprise human immunodeficiency virus (HIV) antigens and gene products selected from the group consisting of gag, pol, and env genes, the Nef protein, reverse transcriptase, string of HIV peptides (Hipo5), a HIVgag-derived p24-PLA HIV gag p24 (gag), and other HIV components, cytomegaloviral antigens, herpes simplex viral antigens, human papilloma virus (HPV) E6 and E7 antigens, or combinations and modifications thereof. Specifically, the antigenic peptides are cancer peptides are selected from tumor associated antigens comprising antigens genitourinary tumors such cervix, uterus, ovarian cancer, vaginal cancer, or combinations and modifications thereof.

The present invention further describes a vaginal vaccine composition for generating an immune response for a prophylaxis, a therapy, amelioration of symptoms or any combinations thereof against one or more vaginal diseases in a human or animal subject comprising: (i) one or more vaginal anti-dendritic cell (DC)-specific antibodies or fragments thereof directed towards one or more specific vaginal DC subsets/macrophages, wherein the antibodies or fragments are directed towards one or more antigens selected from the group consisting of E-cadherin⁺, CD207⁺, CD205⁺CD1c⁺, CD14⁻, DC-ASGPR^{+/-}, CD209^{+/-}, Dectin-1^{+/-}, CD86, CD83, CD209^{+/-}, CD1c⁻, CD14⁺, CD163⁺, LOX-1, CD1d⁺, CD1c⁻, CD14⁻, CD103, β7 integrin, CCR4, CXCR3, and any combinations thereof, (ii) one or more antigenic peptides loaded or chemically coupled with the DC-specific antibodies or fragments thereof, wherein the antigenic peptides are representative of one or more epitopes of the one or more antigens implicated or involved in the vaginal disease or a condition against which the immune response for the prophylaxis, the therapy, amelioration of symptoms, or any combination thereof is desired, and (iii) an optional pharmaceutically acceptable carrier, adjuvants, or any combinations thereof wherein the composition is effective to produce an immune response, for a prophylaxis, a therapy or any combination thereof against the vaginal disease or condition in the human or animal subject.

In one aspect the vaccine is adapted for use in the prophylaxis, the therapy, amelioration of symptoms against a bacterial vaginal infection, a viral vaginal infection, a fungal vaginal infection, one or more sexually transmitted diseases, genitourinary cancers, or any combinations thereof. In another aspect the antigenic peptides comprise antigens produced by organisms selected from the group consisting of Prevotella bivia, Prevotella melaminogenica, Gardnerella vaginalis, Trichomonas vaginalis, Mycoplasma hominis, Mobiluncus species, Neisseria gonorrhoeae, Chlamydia trachomatis, Ureaplasma urealyticus, Candida species, Treponema pallidum, Streptococcus species, and Enterobacteriaceae. In yet another aspect the antigenic peptides comprise human immunodeficiency virus (HIV) antigens and gene products selected from the group consisting of gag, pol, and env genes, the Nef protein, reverse transcriptase, string of HIV peptides (Hipo5), a HIVgag-derived p24-PLA HIV gag p24 (gag), and other HIV components, cytomegaloviral antigens, herpes simplex viral antigens, human papilloma virus (HPV) E6 and E7 antigens, or combinations and modifications thereof. In another aspect the antigenic peptides are cancer peptides are selected from tumor associated antigens comprising antigens from genitourinary tumors such cervix, uterus, ovarian cancer, vaginal cancer, head and neck cancers caused by HPV infections, or combinations and modifications thereof.

The present invention also provides a method for increasing effectiveness of antigen presentation by a vaginal antigen presenting cell (APC) in vitro or in vivo comprising: (i) contacting one or more vaginal dendritic cell (DC) subsets/macrophages with a composition in vitro or administering the composition to a human or animal subject, wherein the composition comprises:

(a) one or more vaginal anti-dendritic cell (DC)-specific antibodies or fragments thereof directed towards one or more specific vaginal DC subsets/macrophages, wherein the antibodies or fragments are directed towards one or more antigens selected from the group consisting of E-cadherin⁺, CD207⁺, CD205⁺ CD1c⁺, CD14⁻, DC-ASGPR^{+/-}, CD209^{+/-}, Dectin-1^{+/-}, CD86, CD83, CD209^{+/-}, CD1c⁻, CD14⁺, CD163⁺, LOX-1, CD1d⁺, CD1c⁻, CD14⁻, CD103, β7 integrin, CCR4, CXCR3, and any combinations thereof; and
(b) one or more native or engineered antigenic peptides chemically coupled or linked to the vaginal DC-specific antibody or fragment to form an antibody-antigen conjugate;
(ii) measuring a level of one or more agents following contact with the one or more vaginal DC subsets/macrophages in vitro or in a biological sample obtained from the human or animal subject, wherein the agents are selected from the group consisting of IFN-γ, TNF-α, IL-5, IL-17, and IL-13, and (iii) determining increased effectiveness of antigen presentation by the conjugate, wherein a change in the level of the one or more agents is indicative of the increase in the effectiveness antigen presentation by the vaginal APCs.

A method for a treatment, a prophylaxis, amelioration of symptoms, or any combinations thereof against one or more diseases or conditions in a human subject is also described herein. The method of the present invention comprises the steps of: (i) identifying the human subject in need of the treatment, the prophylaxis, amelioration of symptoms, or any combinations thereof against the one or more diseases or conditions and (ii) administering a vaccine composition comprising:

(a) one or more vaginal anti-dendritic cell (DC)-specific antibodies or fragments thereof directed towards one or more specific vaginal DC subsets/macrophages, wherein the antibodies or fragments are directed towards one or more antigens selected from the group consisting of E-cadherin⁺, CD207⁺, CD205⁺ CD1c⁺, CD14⁻, DC-ASGPR^{+/-}, CD209^{+/-}, Dectin-1^{+/-}, CD86, CD83, CD209^{+/-}, CD1c⁻, CD14⁺, CD163⁺, LOX-1, CD1d⁺, CD1c⁻, CD14⁻, CD103, β7 integrin, CCR4, CXCR3, and any combinations thereof;
(b) one or more antigenic peptides loaded or chemically coupled with the DC-specific antibodies or fragments thereof, wherein the antigenic peptides are representative of one or more epitopes of the one or more antigens implicated or involved in the disease or the condition against which the immune response for the prophylaxis, the therapy, the amelioration of symptoms, or any combination thereof is desired, and
(c) one or more optional pharmaceutically acceptable carriers and adjuvants wherein the combination of the antibodies and the antigenic peptides is effective to produce an immune response, for a prophylaxis, a therapy, amelioration of symptoms or any combinations thereof against the disease or condition in the human subject.

The present invention in one embodiment describes a method for increasing effectiveness of antigen presentation by one or more dendritic cells (DCs) in a human subject comprising the steps of: isolating one or more DCs or DC subsets from the human subject, wherein the DCs or the DC subsets are isolated from a vaginal tissue or a vaginal mucosa in the human subject, exposing the isolated DCs or DC subsets to activating amounts of an immunostimulatory composition or a vaccine comprising: (i) one or more vaginal anti-dendritic cell (DC)-specific antibodies or fragments thereof directed towards one or more specific vaginal DC subsets/macrophages, wherein the antibodies or fragments are directed towards one or more antigens selected from the group consisting of E-cadherin⁺, CD207⁺, CD205⁺ CD1c⁺, CD14⁻, DC-ASGPR^{+/-}, CD209^{+/-}, Dectin-1^{+/-}, CD86, CD83, CD209^{+/-}, CD1c⁻, CD14⁺ CD163⁺, LOX-1, CD1d⁺, CD1c⁻, CD14⁻, CD103, β7 integrin, CCR4, CXCR3, and any combinations thereof, (ii) one or more antigenic peptides loaded or chemically coupled with the DC-specific antibodies or fragments thereof; and (iii) a pharmaceutically acceptable carrier to form an activated complex; and reintroducing the activated DC complex into the human subject.

One aspect of the method hereinabove comprises the optional step of measuring a level of one or more agents selected from the group consisting of IFN-γ, TNF-α, IL-5, IL-17, and IL-13, wherein a change in the level of the one or more agents is indicative of the increase in the effectiveness of the one or more DCs or DC subsets.

In another aspect the method further comprises the optional steps of: (i) adding one or more Toll-Like Receptor (TLR) agonist which is selected from the group consisting of TLR1, TLR2, TLR3, TLR4, TLR5, TLR6, TLR7, and TLR8 agonists, (ii) adding one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines or combinations and modifications thereof to activated complex prior to exposing the DCs or DC subsets, and (iii) adding one or more optional anti-DC-specific antibodies or fragments thereof selected from antibodies specifically binding to MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASGPR.

In one aspect of the method the antigenic peptides comprise antigens produced by organisms selected from the group consisting of Prevotella bivia, Prevotella melaminogenica, Gardnerella vaginalis, Trichomonas vaginalis, Mycoplasma hominis, Mobiluncus species, Neisseria gonorrhoeae, Chlamydia trachomatis, Ureaplasma urealyticus, Candida species, Treponema pallidum, Streptococcus species, and Enterobacteriaceae, tumor associated antigens comprising antigens from genitourinary tumors such cervix, uterus, ovarian cancer, vaginal cancer, or combinations and modifications thereof, and human immunodeficiency virus (HIV) antigens and gene products selected from the group consisting of gag, pol, and env genes, the Nef protein, reverse transcriptase, string of HIV peptides (Hipo5), a HIV gag-derived p24-PLA HIV gag p24 (gag), and other HIV components, cytomegaloviral antigens, herpes simplex viral antigens, human papilloma virus (HPV) E6 and E7 antigens, or combinations and modifications thereof.

Another embodiment of the present invention provides a method of providing vaginal immunostimulation by activation of one or more vaginal dendritic cell (DC) subsets/macrophages in a human subject for a prophylaxis, a therapy, amelioration of symptoms or any combinations thereof against one or more bacterial, viral, or fungal vaginal infections, one or more sexually transmitted diseases, genitourinary cancers, head and neck cancers caused by HPV infections, or any combinations thereof comprising the steps of: (a) identifying the human subject in need of vaginal immunostimulation for the prophylaxis, the therapy, or a combination thereof against the one or more bacterial, viral, or fungal vaginal infections, one or more sexually transmitted diseases, genitourinary cancers, or any combinations thereof, (b) isolating one or more vaginal DC subsets/macrophages from the human subject, (c) exposing the isolated vaginal DC subsets/macrophages to activating amounts of an immunostimulatory composition or a vaccine comprising:

(i) one or more vaginal anti-dendritic cell (DC)-specific antibodies or fragments thereof directed towards one or more specific vaginal DC subsets/macrophages, wherein the antibodies or fragments are directed towards one or more antigens selected from the group consisting of E-cadherin⁺, CD207⁺, CD205⁺ CD1c⁺, CD14⁻, DC-ASGPR^{+/-}, CD209^{+/-}, Dectin-1^{+/-}, CD86, CD83, CD209^{+/-}, CD1c⁻, CD14⁺, CD163⁺, LOX-1, CD1d⁺, CD1c⁻, CD14⁻, CD103, β7 integrin, CCR4, CXCR3, and any combinations thereof;
(ii) one or more antigenic peptides loaded or chemically coupled with the DC-specific antibodies or fragments thereof; and
(iii) a pharmaceutically acceptable carrier to form an activated complex; and (d) reintroducing the activated DC complex into the human subject.

Yet another embodiment of the present invention provides a vaginal immunostimulatory composition comprising: one or more vaginal anti-dendritic cell (DC)-specific antibodies or fragments thereof directed towards one or more specific vaginal DC subsets/macrophages loaded or chemically coupled with one or more antigenic peptides, wherein the antibodies or fragments are directed towards one or more antigens selected from the group consisting of E-cadherin⁺, CD207⁺, CD205⁺ CD1c⁺, CD14⁻, DC-ASGPR^{+/-}, CD209^{+/-}, Dectin-1^{+/-}, CD86, CD83, CD209^{+/-}, CD1c⁻, CD14⁺, CD163⁺, LOX-1, CD1d⁺, CD1c⁻, CD14⁻, CD103, β7 integrin, CCR4, CXCR3, and any combinations thereof, one or more additional ligands selected from the group consisting of heat-killed bacteria, lipoglycans, lipopolysaccharide, lipoteichoic acids, peptidoglycans, synthetic lipoproteins, zymosan, yeast cell wall components, or combinations and modifications thereof, and one or more optional pharmaceutically acceptable carriers and adjuvants, wherein the composition is effective to produce an immune response, for a prophylaxis, a therapy or any combination thereof in a human or an animal subject.

In one embodiment the present invention relates to a vaccine composition for generating a immune response for a prophylaxis, a therapy, amelioration of symptoms or any combinations thereof against one or more vaginal diseases in a human or an animal subject comprising:

(i) one or more vaginal anti-dendritic cell (DC)-specific antibodies or fragments thereof directed towards one or more specific vaginal DC subsets/macrophages, wherein the antibodies or fragments are directed towards one or more antigens selected from the group consisting of E-cadherin⁺, CD207⁺, CD205⁺ CD1c⁺, CD14⁻, DC-ASGPR^{+/-}, CD209^{+/-}, Dectin-1^{+/-}, CD86, CD83, CD209^{+/-}, CD1c⁻, CD14⁺, CD163⁺, LOX-1, CD1d⁺, CD1c⁻, CD14⁻, CD103, β7 integrin, CCR4, CXCR3, and any combinations thereof;
(ii) one or more antigenic peptides loaded or chemically coupled with the DC-specific antibodies or fragments thereof, wherein the antigenic peptides are representative of one or more epitopes of the one or more antigens implicated or involved in the vaginal disease or a condition against which the immune response for the prophylaxis, the therapy, amelioration of symptoms, or any combination thereof is desired;
(iii) one or more additional ligands selected from the group consisting of heat-killed bacteria, lipoglycans, lipopolysaccharide, lipoteichoic acids, peptidoglycans, synthetic lipoproteins, zymosan, yeast cell wall components, or combinations and modifications thereof; and
(iv) an optional pharmaceutically acceptable carrier, adjuvants, or any combinations thereof wherein the composition is effective to produce an immune response, for a prophylaxis, a therapy or any combination thereof against the vaginal disease or condition in the human or the animal subject.

In one aspect of the composition disclosed hereinabove the vaccine is adapted for use in the prophylaxis, the therapy, amelioration of symptoms against a bacterial vaginal infection, a viral vaginal infection, a fungal vaginal infection, one or more sexually transmitted diseases, genitourinary cancers, or any combinations thereof. In another aspect the antigenic peptides comprise antigens produced by organisms selected from the group consisting of Prevotella bivia, Prevotella melaminogenica, Gardnerella vaginalis, Trichomonas vaginalis, Mycoplasma hominis, Mobiluncus species, Neisseria gonorrhoeae, Chlamydia trachomatis, Ureaplasma urealyticus, Candida species, Streptococcus species, and Enterobacteriaceae. In a specific aspect the antigenic peptides are cancer peptides are selected from tumor associated antigens comprising antigens from genitourinary tumors such cervix, uterus, ovarian cancer, vaginal cancer, head and neck cancers caused by HPV infections, or combinations and modifications thereof. In yet another aspect the anti-DC-specific antibody is humanized. In another aspect the vaccine is adapted for intravaginal administration and enhances production of IL-22 producing CD4⁺ T cells.

Another embodiment of the present invention provides a method for increasing effectiveness of antigen presentation by a vaginal antigen presenting cell (APC) in vitro or in vivo comprising: (a) contacting one or more vaginal dendritic cell (DC) subsets/macrophages with a composition in vitro or administering the composition to a human or animal subject, wherein the composition comprises: (i) one or more vaginal anti-dendritic cell (DC)-specific antibodies or fragments thereof directed towards one or more specific vaginal DC subsets/macrophages, wherein the antibodies or fragments are directed towards one or more antigens selected from the group consisting of E-cadherin⁺, CD207⁺, CD205⁺ CD1c⁺, CD14⁻, DC-ASGPR^{+/-}, CD209^{+/-}, Dectin-1^{+/-}, CD86, CD83, CD209^{+/-}, CD1c⁻, CD14⁺ CD163⁺, LOX-1, CD1d⁺, CD1c⁻, CD14⁻, CD103, ββ7 integrin, CCR4, CXCR3, and any combinations thereof; (ii) one or more native or engineered antigenic peptides chemically coupled or linked to the vaginal DC-specific antibody or fragment to form an antibody-antigen conjugate; and (iii) one or more ligands selected from the group consisting of heat-killed bacteria, lipoglycans, lipopolysaccharide, lipoteichoic acids, peptidoglycans, synthetic lipoproteins, zymosan, yeast cell wall components, or combinations and modifications thereof; (b) measuring a level of one or more agents following contact with the one or more vaginal DC subsets/macrophages in vitro or in a biological sample obtained from the human or animal subject, wherein the agents are selected from the group consisting of IFN-γ, TNF-α, IL-5, IL-17, IL-22, and IL-13, and (c) determining increased effectiveness of antigen presentation by the conjugate, wherein a change in the level of the one or more agents is indicative of the increase in the effectiveness antigen presentation by the vaginal APCs.

In yet another embodiment the instant invention discloses a method for a treatment, a prophylaxis, amelioration of symptoms, or any combinations thereof against one or more diseases or conditions in a human subject comprising the steps of: identifying the human subject in need of the treatment, the prophylaxis, amelioration of symptoms, or any combinations thereof against the one or more diseases or conditions; and administering a vaccine composition comprising:

(i) one or more vaginal anti-dendritic cell (DC)-specific antibodies or fragments thereof directed towards one or more specific vaginal DC subsets/macrophages, wherein the antibodies or fragments are directed towards one or more antigens selected from the group consisting of E-cadherin⁺, CD207⁺, CD205⁺ CD1c⁺, CD14⁻, DC-ASGPR^{+/-}, CD209^{+/-}, Dectin-1^{+/-}, CD86, CD83, CD209^{+/-}, CD1c⁻, CD14⁺ CD163⁺, LOX-1, CD1d⁺, CD1c⁻, CD14⁻, CD103, β7 integrin, CCR4, CXCR3, and any combinations thereof;
(ii) one or more antigenic peptides loaded or chemically coupled with the DC-specific antibodies or fragments thereof, wherein the antigenic peptides are representative of one or more epitopes of the one or more antigens implicated or involved in the disease or the condition against which the immune response for the prophylaxis, the therapy, the amelioration of symptoms, or any combination thereof is desired;
(iii) one or more ligands selected from the group consisting of heat-killed bacteria, lipoglycans, lipopolysaccharide, lipoteichoic acids, peptidoglycans, synthetic lipoproteins, zymosan, yeast cell wall components, or combinations and modifications thereof; and
(iv) one or more optional pharmaceutically acceptable carriers and adjuvants wherein the combination of the antibodies and the antigenic peptides is effective to produce an immune response, for a prophylaxis, a therapy, amelioration of symptoms or any combinations thereof against the disease or condition in the human subject.

One embodiment of the present invention relates to a method for increasing effectiveness of antigen presentation by one or more dendritic cells (DC) subsets/macrophages in a human subject comprising the steps of: isolating one or more DC subsets/macrophages from the human subject, wherein the DCs or the DC subsets are isolated from a vaginal tissue or a vaginal mucosa in the human subject, exposing the isolated DC subsets/macrophages to activating amounts of an immunostimulatory composition or a vaccine comprising: one or more vaginal anti-dendritic cell (DC)-specific antibodies or fragments thereof directed towards one or more specific vaginal DC subsets/macrophages, wherein the antibodies or fragments are directed towards one or more antigens selected from the group consisting of E-cadherin⁺, CD207⁺, CD205⁺ CD1c⁺, CD14⁻, DC-ASGPR^{+/-}, CD209^{+/-}, Dectin-1^{+/-}, CD86, CD83, CD209^{+/-}, CD1c⁻, CD14⁺, CD163⁺, LOX-1, CD1d⁺, CD1c⁻, CD14⁻, CD103, β7 integrin, CCR4, CXCR3, and any combinations thereof; one or more antigenic peptides loaded or chemically coupled with the DC-specific antibodies or fragments thereof; one or more ligands selected from the group consisting of heat-killed bacteria, lipoglycans, lipopolysaccharide, lipoteichoic acids, peptidoglycans, synthetic lipoproteins, zymosan, yeast cell wall components, or combinations and modifications thereof; and a pharmaceutically acceptable carrier to form an activated complex; and reintroducing the activated DC complex into the human subject.

Finally, the present invention discloses a method of providing vaginal immunostimulation by activation of one or more vaginal dendritic cell (DC) subsets/macrophages in a human subject for a prophylaxis, a therapy, amelioration of symptoms or any combinations thereof against one or more bacterial, viral, or fungal vaginal infections, one or more sexually transmitted diseases, genitourinary cancers, or any combinations thereof comprising the steps of: (a) identifying the human subject in need of vaginal immunostimulation for the prophylaxis, the therapy, or a combination thereof against the one or more bacterial, viral, or fungal vaginal infections, one or more sexually transmitted diseases, genitourinary cancers, or any combinations thereof; (b) isolating one or more vaginal DC subsets/macrophages from the human subject; (c) exposing the isolated vaginal DC subsets/macrophages to activating amounts of an immunostimulatory composition or a vaccine comprising: i) one or more vaginal anti-dendritic cell (DC)-specific antibodies or fragments thereof directed towards one or more specific vaginal DC subsets/macrophages, wherein the antibodies or fragments are directed towards one or more antigens selected from the group consisting of E-cadherin⁺, CD207⁺, CD205⁺ CD1c⁺, CD14⁻, DC-ASGPR^{+/-}, CD209^{+/-}, Dectin-1^{+/-, CD}86, CD83, CD209^{+/-}, CD1c⁻, CD14⁺ CD163⁺, LOX-1, CD1d⁺, CD1c⁻, CD14⁻, CD103, β7 integrin, CCR4, CXCR3, and any combinations thereof, ii) one or more antigenic peptides loaded or chemically coupled with the DC-specific antibodies or fragments thereof, iii) one or more ligands selected from the group consisting of heat-killed bacteria, lipoglycans, lipopolysaccharide, lipoteichoic acids, peptidoglycans, synthetic lipoproteins, zymosan, yeast cell wall components, or combinations and modifications thereof; and iv) a pharmaceutically acceptable carrier to form an activated complex, and (d) reintroducing the activated DC complex into the human subject.

The method of the present invention as disclosed above further comprising the optional step of measuring a level of one or more agents selected from the group consisting of IFN-γ, TNF-α, IL-5, IL-17, IL-22, and IL-13, wherein a change in the level of the one or more agents is indicative of immunostimulation. In specific aspects of the method above the ligand is zymosan and the DC-specific antibody is humanized. In yet another aspect the reintroduction of the activated DC complex is done intravaginally.

Yet another embodiment is a method of performing a clinical trial to evaluate a candidate drug believed to be useful in treating vaginal diseases, the method comprising: a) isolating at least one subset of antigen presenting cells (APCs), wherein the APCs possess a distinct phenotype, wherein the subset is selected from at least one of Langerhans cells (LCs) E-cadherin+CD207+CD205+, vaginal CD1c+CD14−DC−ASGPR+CD209+/−Dectin-1+/− dendritic cells (DCs), vaginal CD1c+CD14+CD209+/−DC-ASGPR+/−DCs, CD1c-CD14+CD163+CD209+DC-ASGPR+/−Dectin-1+/−LOX-1+CD1d+ macrophages, or vaginal CD1c-CD14− DCs, b) determining the T cell activating activity of the antigen presenting cells isolated from the patient; c) administering a candidate drug to a first subset of the patients, and a placebo to a second subset of the patients; a comparator drug to a second subset of the patients; or a drug combination of the candidate drug and another active agent to a second subset of patients; d) repeating step a) after the administration of the candidate drug or the placebo, the comparator drug or the drug combination; and e) monitoring the T cell activating activity of the antigen presenting cells, wherein a statistically significant change in T cell activating activity indicates that the candidate drug is useful for treating the vaginal disease.

### BRIEF DESCRIPTION OF THE DRAWINGS

For a more complete understanding of the features and advantages of the present invention, reference is now made to the detailed description of the invention along with the accompanying figures and in which:

FIGS. 1A-1E show that human vaginal mucosa contains at least four major subsets of APCs:

FIG. 1A is a flow cytometry analysis of cells in human vaginal tissues after enzymatic digestion. Live HLA⁻DR⁺ cells were gated (left panel) and Langerin⁺ cells were gated (middle panel). HLA⁻ DR⁺Langerin⁻ cells were further divided into four groups based on CD1c and CD14 expression (right panel). Five population of HLA⁻DR⁺ were defined (gate I: Langerin⁺; and among the Langerin⁻: gate II: CD1c⁺CD14⁻; gate III: CD1c⁺CD14′; gate IV: CD1c⁻CD14⁺ and gate V: CD1c⁻CD14⁻), FIG. 1B shows cell morphology of FACS-sorted subpopulations of vaginal cells (×100, bars are 20 μm), FIG. 1C shows frozen tissue sections were stained for Langerin (green); CD14 (red); CD1c (light blue) and cell nuclei (dark blue) (×20 bar is 100 μm). Data are representative of six independent studies using tissue sections from different donors, FIG. 1D shows the percentage of HLA⁻DR⁺ cells in total vaginal cells prepared by enzymatic digestion, and FIG. 1E shows the percentage of vaginal cell subsets (Langerin⁺, I, II, III, and IV in a) in HLA⁻DR⁺ cells. Each dot in FIGS. 1D and 1E indicates data generated with tissues from different donors;

FIG. 1F shows the isotype controls for FIG. 1C. Vaginal tissue sections from the donor tested in FIG. 1C were stained with isotype control antibodies (×20, bar 100 μm);

FIGS. 2A-2C show the phenotype of the subsets of vaginal APCs: FIG. 2A is a flow-cytometry analysis of vaginal APC subsets. Tissues were digested with enzymes and single cell suspension was stained with indicated antibodies and gated as in FIG. 1A. Individual subsets of APCs were analyzed for surface expression of CD1a, CD11c, CD86, CD83, CD163 and E-cadherin. Gray histograms represent isotype controls, FIGS. 2B and 2C show frozen tissue sections stained for CD1a, CD1c, and CD14 (2B) and CD1c, CD14 and CD163 (2C) (×20, bar is 100 μm);

FIGS. 2D and 2E show the isotype controls for FIGS. 2B and 2C. Vaginal tissue sections from the same donors tested in FIGS. 2A and 2B were stained with isotype control antibodies (×20, bar 100 μm). Isotype controls for FIG. 2B (2D) and FIG. 2C (2E);

FIGS. 3A-3F show C-type lectin-like receptor and CD1d expression in APC subsets localized in the vagina. Frozen tissue sections were stained for DEC-205, CD1c and Langerin (FIG. 3A), DC-SIGN, CD1c and CD14 (FIG. 3B), Dectin-1, CD1c, and CD163 (FIG. 3C), ASGPR, CD1c and CD14 (FIG. 3D), LOX-1, CD1c, and CD163 (FIG. 3E), and CD1d, CD1c, and CD14 (FIG. 3F) (×20, bar is 100 μm). For each panel (FIGS. 3A-3F), tissue sections from at least three different donors were tested and representative data are presented;

FIGS. 3G-3L shows isotype controls for FIGS. 3A-3F. Vaginal tissue sections from the same donors tested in FIGS. 3A-3F were stained with isotype control antibodies (×20, bar 100 μm);

FIG. 4 shows the analysis of chemokine receptor and β7 integrin expression on the surface of vaginal APC subsets. Single cell suspensions of whole vaginal mucosa were stained for CCR2, CCR4, CCR5, CCR6, CCR7, CXCR4, CX3CR1 and β7 integrin. Gray histograms represent isotype controls. Subsets of APCs were gated as in FIG. 1A. Six independent studies using cells from different donors were performed and representative data from one study are presented;

FIGS. 5A-5C shows the functional specialization of vaginal APC subsets for eliciting naïve CD4⁺ T cell responses. FACS-sorted CFSE-labeled allogeneic naïve T cells were co-cultured for 7 days with different numbers of vaginal APCs (left panel) or 2×10³ IFNDCs (right panel). FIG. 5A shows live CD4⁺ T cells were gated and CD4⁺ T cell proliferation was assessed by measuring CFSE dilution. Data are mean±SD of 4 independent studies with duplicates (* indicates p<0.05; ANOVA test), FIG. 5B after 7 days, T cells were stimulated with PMA and ionomycin in the presence of brefeldin A. Cells were then stained for intracellular IFNγ, TNFα, IL-13 and IL-5. Six independent studies using cells from different donors showed similar data. Data from one representative study are presented, and FIG. 5C Boolean gate analysis of CD4⁺ T cell populations expressing different cytokines CD4⁺ T cells expressing IFNγ, IL-13, and IL-5 (left panels) and CD4⁺ T cells expressing IFNγ, IL-13 and TNFα (right panels) are separately analyzed. IFNγ, IL-13, and IL-5 (N=6) and IFNγ, IL-13, and TNFα (N=3);

FIG. 5D shows vaginal LCs and CD1c⁺CD14⁻ DCs polarize naïve CD4⁺ T cells toward Th2-type, while CD1c⁻CD14⁺ macrophages polarize them toward Th1-type. CFSE-labeled allogeneic naïve T cells were co-cultured for 7 days with FACS-sorted vaginal APC subsets or in vitro generated monocyte-derived IFNDCs. T cells were restimulated with PMA and ionomycin in the presence of BFA. Cells were stained for intracellular cytokine expressions. Live CD4⁺ T cells were gated and expression of IFNγ, TNFα, IL-13, and IL-5 were analyzed. Each dot represents data from independent studies using APCs from different donors. * indicates p<0.05 (ANOVA test);

FIG. 5E shows vaginal LCs and CD1c⁺CD14⁻ DCs promote Th2, while IFNDCs promote Th1-type CD4⁺ T cell differentiation. CFSE-labeled allogeneic naïve T cells were co-cultured for 7 days with FACS-sorted vaginal APC subsets or in vitro generated monocyte-derived IFNDCs. CFSE low CD4⁺ T cells were sorted by FACS and restimulated with anti-CD3 and anti-CD28 for 48 h. Cytokines in culture supernatants were measured by Luminex. Data from one representative study with duplicates are presented;

FIGS. 6A-6D shows that subsets of vaginal APCs display distinct functions in eliciting CD8⁺ T cell responses. CFSE-labeled allogeneic naïve T cells were co-cultured for 7 days with vaginal APCs or IFNDCs: FIG. 6A shows CD8⁺ T cell proliferation was assessed by measuring CFSE dilution. Data are mean±SD of two independent studies with duplicates (* indicates p<0.05; ANOVA test), FIG. 6B after 7 days, T cells were stimulated with PMA and ionomycin in the presence of brefeldin A, and then stained for intracellular IFNγ, TNFα, and IL-5 expression. Six independent studies using APCs from different donors showed similar results. Representative data from one study presented, FIG. 6C shows the percentage of CD8⁺ T cells expressing IFNγ⁺TNFα⁺, IFNγ⁺TNFα⁻, and IFNγ⁻TNFα⁺. Data (mean±SD) from three independent studies using APCs from different donors are summarized, and FIG. 6D shows the percentage of CD8⁺ T cells expressing IFNγ⁺IL-5⁺, IFNγ⁻IL-5⁺, and IFNγ⁺IL-5⁻. Data (mean±SD) from six independent studies using APCs from different donors are summarized. (FIGS. 6C and 6D) (* indicates p<0.05; ANOVA test);

FIG. 6E shows vaginal CD1c⁺CD14⁻ DCs and LCs induce IL-5-producing CD8⁺ T cells. CFSE-labeled allogeneic naïve T cells were co-cultured for 7 days with FACS-sorted vaginal APC subsets or in vitro generated monocyte-derived IFNDCs. T cells were restimulated with PMA and ionomycin in the presence of BFA. Cells were stained for intracellular cytokine expressions. Live CD8⁺ T cells were gated and expression of IFNγ, TNFα and IL-5 were analyzed. Each dot represents data from independent studies using APCs from different donors. * indicates p<0.05 (ANOVA test);

FIGS. 7A-7D show that Zymosan can enhance vaginal LC-mediated IL-22-producing CD4⁺ T cell responses. CFSE-labeled naïve T cells were co-cultured for 7 days with vaginal APCs or IFNDCs in the presence or absence of 10 μg/ml zymosan: FIG. 7A CD4⁺ T cell proliferation was assessed by measuring CFSE dilution. Data are mean±SD of 10 independent studies using APCs from different donors, FIG. 7B after 7 days, CD4⁺ T cells were restimulated with PMA and ionomycin in the presence of brefeldin A, and then stained for intracellular IL-22. Ten independent studies using DCs from different donors showed similar results. Representative data from one study is presented, FIG. 7C shows summarized data generated with LCs from FIG. 7B each line represents the data from an independent study using APCs from ten different donors, and FIG. 7D shows the frequency of Th1, Th2 and Th17 cells among the IL-22-producing CD4⁺ T cells after co-culture with zymosan-activated LCs. Five independent studies using DCs from different donors showed similar results. Representative data from one study is presented (* indicates p<0.05; Student t-test);

FIGS. 7E-7G show CD4⁺ T cell responses induced by zymosan-activated vaginal APCs. CFSE-labeled allogeneic naïve T cells were co-cultured for 7 days with FACS-sorted vaginal APC subsets or in vitro generated monocyte-derived IFNDCs, in absence or presence of 10 μg/ml of zymosan. T cells were restimulated with PMA and ionomycin in the presence of BFA, and then stained for intracellular IFNγ (FIG. 7E), IL-5 (FIG. 7F), and IL-17 (FIG. 7G). Combined data of 10 independent studies using APCs from different donors are presented;

FIGS. 8A-8F show that Vaginal APCs can induce CD103 and CCR4, which are found to be expressed on T cells in the vagina: FIG. 8A CD103 expression analysis of CD4⁺ (top panel) and CD8⁺ (bottom panel) T cells from vaginal mucosal tissues, FIGS. 8B and 8C show frozen tissue sections were stained for CD3, CD4 and CD103 (8B) and CD3, CD8, CD103 (8C) (×20, bar is 100 μm), FIG. 8D show the expression of CD103 on naïve CFSE-labeled CD4⁺ (top panel) and CD8⁺ T cells (bottom panel) co-cultured for 7 days with vaginal APCs or IFNDCs, FIG. 8E shows CCR4 expression on CD4⁺ (top panel) and CD8⁺ (bottom panel) T cells from vaginal mucosal tissues, and FIG. 8F shows expression of CCR4 on naïve CFSE-labeled CD4⁺ (top panel) and CD8⁺ T cells (bottom panel) co-cultured for 7 days with vaginal APCs or IFNDCs. (FIGS. 8A-8C and 8E) T cells in the vagina from five donors showed similar results. (FIGS. 8D and 8F) show four independent studies using APCs from different donors showed similar results. Representative data from one study is presented;

FIGS. 8G and 8H show isotype controls for FIGS. 8B and 8C. Vaginal tissue sections from the same donor tested in FIGS. 8B and 8C were stained with isotype control antibodies (×20, bar 100 μm); and

FIGS. 9A-9D show vaginal APCs induce the expression of CXCR3 and β7 integrin on allogeneic naïve T cells: FIG. 9A is a flow-cytometry analysis of β7 integrin expression on CD4⁺ and CD8⁺ T cells sorted from vaginal samples, FIG. 9B shows the expression of β7 integrin on naïve CFSE-labeled CD4⁺ and CD8⁺ T cells co-cultured for 7 days with vaginal APCs or in vitro generated monocyte-derived IFNDCs. CFSE⁺ cells were gated, FIG. 9C show the flow-cytometry analysis of CXCR3 expression on CD4⁺ and CD8⁺ T cells sorted from vaginal samples, and FIG. 9D shows the expression of CXCR3 on naïve CFSE-labeled CD4⁺ and CD8⁺ T cells co-cultured for 7 days with vaginal APCs or in vitro generated monocyte-derived IFNDCs. CFSE^{low} cells were gated. One representative study out of three independent studies is presented.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “Antigen Presenting Cells” (APC) refers to cells that are capable of activating T cells, and include, but are not limited to, certain macrophages, B cells and dendritic cells. “Dendritic cells” (DCs) refers to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells). These cells can be isolated from a number of tissue sources, and conveniently, from peripheral blood, as described herein. Dendritic cell binding proteins refers to any protein for which receptors are expressed on a dendritic cell. Examples include GM-CSF, IL-1, TNF, IL-4, CD40L, CTLA4, CD28, and FLT-3 ligand.

The term “vaccine composition” as used in the present invention is intended to indicate a composition which can be administered to humans or to animals in order to induce an immune system response; this immune system response can result in a production of antibodies or simply in the activation of certain cells, in particular antigen-presenting cells, T lymphocytes and B lymphocytes. The vaccine composition can be a composition for prophylactic purposes or for therapeutic purposes or both.

The term “adjuvant” refers to a substance that enhances, augments, or potentiates the host's immune response to a vaccine antigen.

The term “antibodies” refers to immunoglobulins, whether natural or partially or wholly produced artificially, e.g. recombinant. An antibody may be monoclonal or polyclonal. The antibody may, in some cases, be a member of one or a combination immunoglobulin classes, including: IgG, IgM, IgA, IgD, and IgE. The invention includes also variants and other modification of an antibody (or “Ab”) of fragments thereof. As used herein, the term “antibodies or fragments thereof,” includes whole antibodies or fragments of an antibody, e.g., Fv, Fab, Fab′, F(ab′)2, Fc, and single chain Fv fragments (ScFv) or any biologically effective fragments of an immunoglobulins that binds specifically to an antigen or target. Antibodies from human origin or humanized antibodies have lowered or no immunogenicity in humans and have a lower number or no immunogenic epitopes compared to non-human antibodies. Antibodies and their fragments will generally be selected to have a reduced level or no antigenicity in humans.

As used herein, the terms “Ag” or “antigen” refer to a substance capable of either binding to an antigen binding region of an immunoglobulin molecule or of eliciting an immune response, e.g., a T cell-mediated immune response by the presentation of the antigen on Major Histocompatibility Antigen (MHC) cellular proteins. As used herein, “antigen” includes, but is not limited to, antigenic determinants, haptens, and immunogens, which may be peptides, small molecules, carbohydrates, lipids, nucleic acids or combinations thereof. The skilled immunologist will recognize that when discussing antigens that are processed for presentation to T cells, the term “antigen” refers to those portions of the antigen (e.g., a peptide fragment) that is a T cell epitope presented by MHC to the T cell receptor. When used in the context of a B cell mediated immune response in the form of an antibody that is specific for an “antigen”, the portion of the antigen that binds to the complementarity determining regions of the variable domains of the antibody (light and heavy) the bound portion may be a linear or three-dimensional epitope. In certain cases, the antigens delivered by the vaccine or a fusion protein and are internalized and processed by antigen presenting cells prior to presentation, e.g., by cleavage of one or more portions of the antibody or fusion protein.

As used herein, the term “antigenic peptide” refers to that portion of a polypeptide antigen that is specifically recognized by either B-cells or T-cells. B-cells respond to foreign antigenic determinants via antibody production, whereas T-lymphocytes are the mediate cellular immunity. Thus, antigenic peptides are those parts of an antigen that are recognized by antibodies, or in the context of an MHC, by T-cell receptors.

As used herein, the term “epitope” refers to any protein determinant capable of specific binding to an immunoglobulin or of being presented by a Major Histocompatibility Complex (MHC) protein (e.g., Class I or Class II) to a T-cell receptor. Epitopic determinants are generally short peptides 5-30 amino acids long that fit within the groove of the MHC molecule that presents certain amino acid side groups toward the T cell receptor and has certain other residues in the groove, e.g., due to specific charge characteristics of the groove, the peptide side groups and the T cell receptor. Generally, an antibody specifically binds to an antigen when the dissociation constant is 1 mM, 100 nM, or even 10 nM.

The term “gene” is used to refer to a functional protein, polypeptide or peptide-encoding unit. As will be understood by those in the art, this functional term includes genomic sequences, cDNA sequences or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

As used herein, the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA) or analogs of naturally-occurring nucleotides (e.g., a-enantiomeric forms of naturally-occurring nucleotides) or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

As used in this application, the term “amino acid” refers to the one of the naturally occurring amino carboxylic acids of which proteins are comprised. The term “polypeptide” as described herein refers to a polymer of amino acid residues joined by peptide bonds, whether produced naturally or synthetically. Polypeptides of less than about 10 amino acid residues are commonly referred to as “peptides.” A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

As used herein, the term “in vivo” refers to being inside the body. The term “in vitro” used as used in the present application is to be understood as indicating an operation carried out in a non-living system.

The term “tissue sample” (the term “tissue” is used interchangeably with the term “tissue sample”) should be understood to include any material composed of one or more cells, either individual or in complex with any matrix or in association with any chemical. The definition shall include any biological or organic material and any cellular subportion, product or by-product thereof. The definition of “tissue sample” should be understood to include without limitation sperm, eggs, embryos and blood components. Also included within the definition of “tissue” for purposes of this invention are certain defined acellular structures such as dermal layers of skin that have a cellular origin but are no longer characterized as cellular.

As used herein, “pharmaceutically acceptable carrier” refers to any material that when combined with an immunoglobulin (Ig) fusion protein of the present invention allows the Ig to retain biological activity and is generally non-reactive with the subject's immune system. Examples include, but are not limited to, standard pharmaceutical carriers such as a phosphate buffered saline solution, water, emulsions such as an oil/water emulsion, and various types of wetting agents. Certain diluents may be used with the present invention, e.g., for aerosol or parenteral administration, that may be phosphate buffered saline or normal (0.85%) saline.

The terms “administration of” or “administering a” compound as used herein refers to providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The terms “effective amount” or “therapeutically effective amount” as used herein should be understood to indicate the amount of the subject compound that will elicit the biological or medical response of a tissue, system, animal or human that is being sought by the researcher, veterinarian, medical doctor or other clinician.

As used herein, the term “treatment” or “treating” includes any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

The present invention describes the discovery of at least four major subsets of myeloid-originated antigen-presenting cells (APCs) that possess distinct phenotypes and functions in directing immune responses in the human vaginal mucosa. Langerhans cells (LCs: E-cadherin⁺CD207⁺CD205^{+x}), CD1c⁺CD14⁻ DCs (DC-ASGPR⁺CD209^{+/-}Dectin-1^{+/-}), and CD1⁺CD14⁺ DCs (CD209^{+/-}DC⁻ASGPR^{+/-}) all express high levels of CD11c, CD83, and CCR6, and are more potent than CD1c⁻CD14⁺ macrophages (CD163⁺CD209^{+/-}DC⁻ASGPR^{+/-}Dectin-1^{+/-LOX-}1⁺CD1d⁺) at eliciting naïve T cell proliferation. LCs and CD1c⁺CD14⁻ DCs polarize naïve T cells toward Th2-type, whereas CD1c⁺CD14⁺ DCs and CD1c⁻CD14⁺ macrophages polarize them toward Th1-type.

Furthermore, the present invention found that LCs activated with zymosan, a cell wall component of commensal yeasts in the vagina, enhances Th22-type T cell responses that contribute to innate immunity and maintenance of epithelial barriers. Studies conducted by the present inventors show that vaginal DCs efficiently induce the mucosal-homing receptors, CD103, β7 integrin, CCR4, and CXCR3, which are expressed on T cells in the human vagina.

Sexually transmitted diseases (STDs) cause high morbidity and mortality, and they still remain a major disease burden worldwide. Despite the fact that the human vagina represents a major route of entry and infection site for sexually transmitted pathogens, the immunology of the human vaginal mucosa is still poorly understood (Iwasaki, 2010; Mestecky et al., 2009).

Data from early studies in animal models showed that the immune system in the vagina exhibits several features distinct from other mucosal tissues, including the absence of organized lymphoepithelial inductive sites (Iwasaki, 2010; Mestecky et al., 2005). However, the vaginal mucosa, a type II mucosa, is covered with stratified squamous epithelium (Iijima et al., 2008b; Iwasaki, 2007; Iwasaki, 2010; Mestecky et al., 2005), which shares common features with the skin. For example, Langerhans cells (LCs) are found in the epithelial layer and CD11c⁺ dendritic cells (DCs) in the submucosa (Iwasaki, 2007; Iwasaki, 2010; Mestecky et al., 2009). In addition, the murine vagina contains four subgroups of LCs characterized by the expression of MHC class II molecules and other cell surface markers (I-A⁺F4/80⁺, I-A⁺F4/80−, I-A⁺CD205⁺, and I-A⁺CD205−) (Parr and Parr, 1991). None of these populations expresses CD11b, MOMA-1, or MOMA-2. More recently, DC-SIGN⁺CCR5⁺ submucosal DCs have been reported in animal models (Hu et al., 1998; Iijima et al., 2008b; Jameson et al., 2002), but no further information is available particularly for the human vaginal mucosa.

Functional diversity of local tissue-resident DC subsets has been previously described (Allan et al., 2006; den Haan et al., 2000; Huang et al., 2000; Johansson and Kelsall, 2005; Villadangos and Schnorrer, 2007). For the vagina, both lymphoid DCs and DCs from the vagina contribute to priming CD4⁺ and CD8⁺ T cell responses (Lee et al., 2009). More importantly, tissue-resident DCs play important roles in eliciting protective immunity in the vaginal mucosa, particularly when mice were intra-vaginally infected with HSV-2 (Lee et al., 2009). Other studies have also shown that intra-vaginal administration of vaccines (Kwant and Rosenthal, 2004; Lindqvist et al., 2009), including non-replicating antigens (Echchannaoui et al., 2008; Haneberg et al., 1994; Kozlowski et al., 1997; Wassen et al., 1996), can mount mucosal immunity in the vagina. The ability of the female genital tract to initiate immune responses is further supported by the data from studies performed in mice (Hedges et al., 1998; Rosenthal and Gallichan, 1997). Antigen-specific lymphocytes were also found in iliac lymph nodes after intra-vaginal immunization (Gupta et al., 2005). More recently, Zhao et al. (Zhao et al., 2003) have shown that vaginal submucosal DCs, but not LCs, induce protective Th1 responses to HSV-2 infection in mice. Taken together, these data suggest that APC subsets, including DCs, in the human vaginal mucosa might possess distinct functions in directing immune responses in the female genital tract.

To study the immunology of the human vagina, the present inventors characterized subsets of APCs that localize to vaginal tissues by both flow cytometry and immunofluorescence methods. Individual subsets of APCs were further characterized by assessing expression levels of costimulatory molecules, lectin-like receptors (LLRs; including DEC205, DC-SIGN, Dectin-1, LOX-1, and DC-ASGPR), chemokine receptors and other mucosal homing receptors. Distinct patterns of LLR expressed on different subsets of APCs are associated with their immunological functions (Brown, 2006; Delneste et al., 2002; Dudziak et al., 2007; Figdor et al., 2002; Geijtenbeek et al., 2004). T is further demonstrated that human vaginal APC subsets have distinct functions in directing T cell responses by polarizing CD4⁺ and CD8⁺ T cell responses and by inducing chemokine and other mucosal homing receptors, which are found to be expressed on T cells localized in the human vaginal mucosa.

Vaginal tissues were obtained from patients (26-88 years old) who have undergone vaginal repair surgeries under a protocol that has been approved by the Institutional Review Board of Baylor Research Institute. Patients were not infected with HIV, HCV, or TB. All tissues tested were not inflamed.

Enzymatic digestion of vaginal mucosa: Tissues were dissected free from fat, cut in small pieces (1-5 mm²) and digested 3 h at 37° C. with 0.6 unit/ml Dispase II, 2 mg/ml collagenase D (both from Roche Applied Science, Indianapolis, Ind.), 200 μg/ml DNase I (Invitrogen, Carlsbad, Calif.), 20 units/ml hyaluronidase (Sigma Aldrich, St. Louis, Mo.) in RPMI 1640 (Invitrogen) supplemented with 25 mM HEPES buffer (Invitrogen), 2 mM L-glutamine (Sigma), 1% nonessential amino-acids (Sigma), 1 mM sodium pyruvate (Sigma), antibiotic/antimycotic (Invitrogen), and 5% FCS (HyClone, Logan, Utah). Cell suspensions were filtered consecutively on 100 μm, 70 μm and 40 μm cell strainers (BD Biosciences, San Jose, Calif.) and washed.

Cell phenotype: Cells were stained with 7-AAD (Biolegend, San Diego, Calif.), anti-HLA-DR-AF700 (Biolegend), anti-Langerin PE (Beckman Coulter, Brea, Calif.) or anti-Langerin AF488 (in house), anti-CD1c-AF647 (Biolegend), CD14-eFluor450 (eBiocience, San Diego, Calif.) and anti-CD1a, anti-CD11c, anti-CD83, anti-CD86, anti-CCR6, anti-ecadherin antibodies (Abs) from Biolegend, anti-f37 integrin and anti-DCSIGN Abs from BD Biociences, anti-CCR2, anti-CCR4, anti-CCR5, anti-CCR7, and anti-CXCR4Abs from R&D Systems, anti-CD163 from BMA Biomedicals (Switzerland) and anti-CX3CR1 from MBL International (Woburn, Mass.). Phenotypes of vaginal APCs were analyzed by flow cytometry on an LSR II (BD Biosciences). Anti-CD103 and anti-CCR4Abs used for T cell phenotyping were from eBioscience and R&D Systems, respectively.

Isolation of vaginal APCs by in vitro migration: Tissues were dissected free from fat, cut in small pieces approximately 1 cm², and incubated in PBS containing 2 mM EDTA and antibiotic/antimycotic solution overnight at 4° C. or 2 h at 37° C. Epithelium and submucosa were then separated using forceps. Submucosa was cut in smaller pieces (1-5 mm²). Epithelial sheets and submucosal pieces were incubated for 2 days at 37° C. in RPMI 1640 supplemented with 25 mM HEPES buffer, 2 mM L-glutamine, 1% nonessential amino-acids, 1 mM sodium pyruvate, antibiotic/antimycotic, and 10% FCS. Migratory cells were recovered, filtered consecutively on 100 μm, 70 μm and 40 μm cell strainers and washed. Cells were stained with 7-AAD, anti-HLA-DR-AF700, anti-Langerin-PE, anti-CD1c-FITC (Invitrogen) and CD14-eFluor450. HLA-DR⁺ cells were gated and then Langerin⁺, CD1c⁺CD14⁻, CD1c⁺CD14⁺, and CD14⁺CD1c⁻ cells were sorted by FACS Aria II (BD Biosciences).

Morphology of vaginal APCs: Giemsa staining of sorted vaginal APCs was done using the Diff-Quik™ Stain Set according to the manufacturer's protocol (Siemens Healthcare Diagnostics, Newark, Del.). Images were acquired using an Olympus BX60 microscope with Planapo 100×/1.4oil objective and a Nikon DXM1200C digital color camera with Nikon NIS Elements F Version 2.30 software.

Preparation of T cells and monocyte-derived in vitro-generated IFNDCs: Peripheral blood mononuclear cells (PBMCs) from healthy volunteers were fractionated by elutriation, under a protocol that has been approved by the Institutional Review Board of Baylor Research Institute. IFNDCs were generated by culturing monocytes from healthy donor in serum free medium (Cellgenix, Freiburg, Germany) supplemented with GM-CSF (100 ng/ml) and IFNγ (500 U/ml) (IFNDCs). The medium was replenished with cytokines on day 1 for IFNDCs. IFNα and GM-CSF were from the Pharmacy at Baylor University Medical Center (Dallas, Tex.). T cells were enriched using EasySep Human T Cell Enrichment Kit (Stemcell, Vancouver, Canada). Naïve (CD45RA⁺CD45RO-CCR7⁺) T cells (purity>99.2%) were sorted on FACS Aria II.

Vaginal APC-mediated T cell responses: 1×10⁵ CFSE-labeled purified naïve CD4⁺ T cells and 0.5×10⁵ CFSE-labeled purified naïve CD8⁺ T cells were co-cultured with 2×10³ (or indicated number) APCs in RPMI 1640 supplemented with 25 mM HEPES buffer, 2 mM L-glutamine, 1% nonessential amino-acids, 1 mM sodium pyruvate, 50 units/ml penicillin, 50 μg/ml streptomycin and 10% AB serum (GemCell, West Sacramento, Calif.). In some studies, 10 μg/ml Zymosan (Invivogen, San Diego, Calif.) was added to the culture. After 7 days, cells were stained with anti-CD4 APC-Cy7 (Biolegend), anti-CD8 Pacific Blue (Biolegend) and LIVF/DEAD® Fixable Dead Cell Stain Kit (Invitrogen), and T cell proliferation was tested by measuring CFSE-dilution. For cytokine expression analysis, T cells were restimulated with 100 ng/ml phorbol 12-myristate 13-acetate (PMA; Sigma) and 1 μg/ml ionomycin (Sigma) for 6 h in the presence of GolgiPlug (BD Biosciences). They were then stained with anti-CD4, anti-CD8, LIVF/DEAD® Fixable Dead Cell Stain Kit, anti-IFNγ, anti-IL-5, anti-TNFα, anti-IL-13 (all from Biolegend), anti-IL-22 (R&D Systems) and/or anti-IL17 (eBioscience) antibodies labeled with fluorescent dyes. Intracellular staining was performed using BD Cytofix/Cytoperm™ Fixation/Permeabilization Solution Kit according to manufacturer's protocol. Cytokine expression was next detected by flow cytometry (FACS Canto II, BD Biosciences).

Immunofluorescence: Cryo-sections were fixed in cold acetone, dried and blocked for non-specific fluorescence with Fc Receptor Block and Background Buster (Innovex Biosciences, Richmond, Calif.). Sections were stained with the indicated antibodies and then subsequently stained with DAPI (Invitrogen). Digital images were taken using an Olympus BX51 with a Planapo20/0.7 or Planapo40/0.95 objective, a Roper Coolsnap HQ camera and Metamorph software (Molecular Devices, Sunnyvale, Calif.). Images were acquired using the same exposures for antibody and isotype staining and identical scaling was applied. Confocal images were taken with the Leica SP1 and Planapo63/1.32 objective.

Statistical Analysis: Statistical significance was determined using the Student's t-test and ANOVA test using Prism 5 software (GraphPad Software Inc, La Jolla, Calif.). Significance was set at P<0.05.

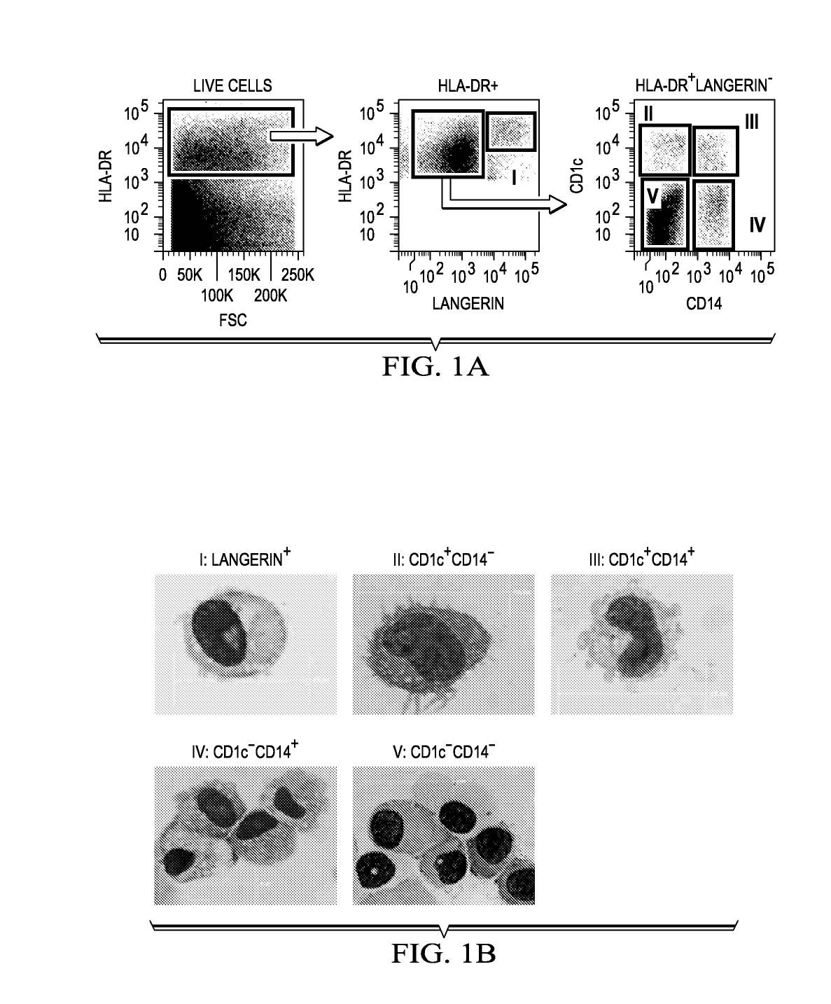

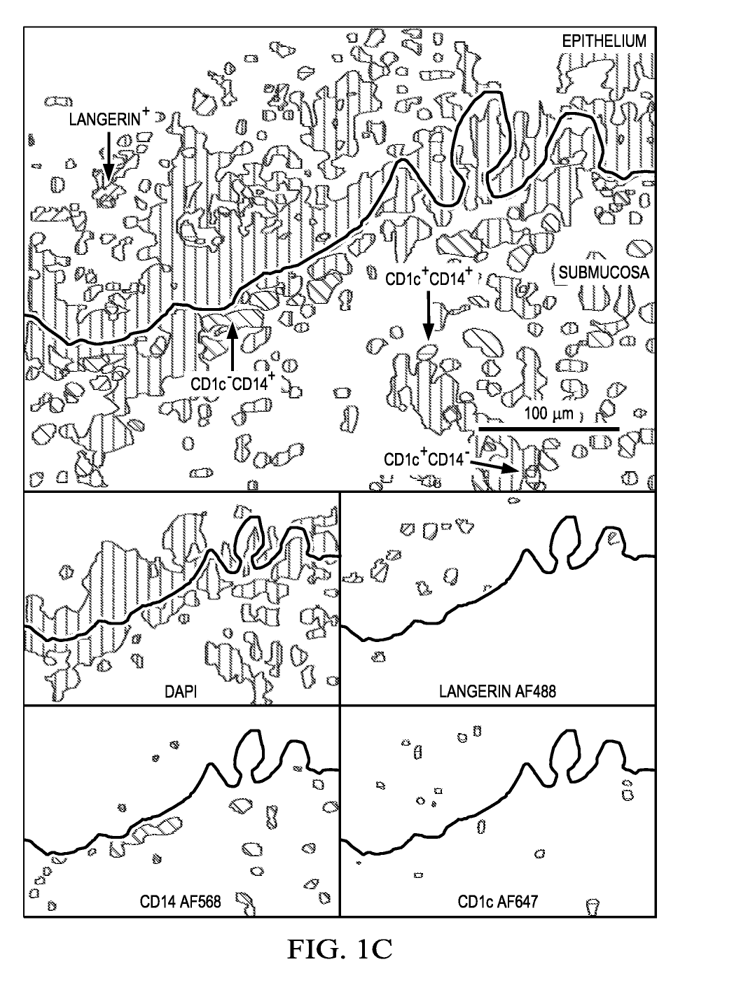

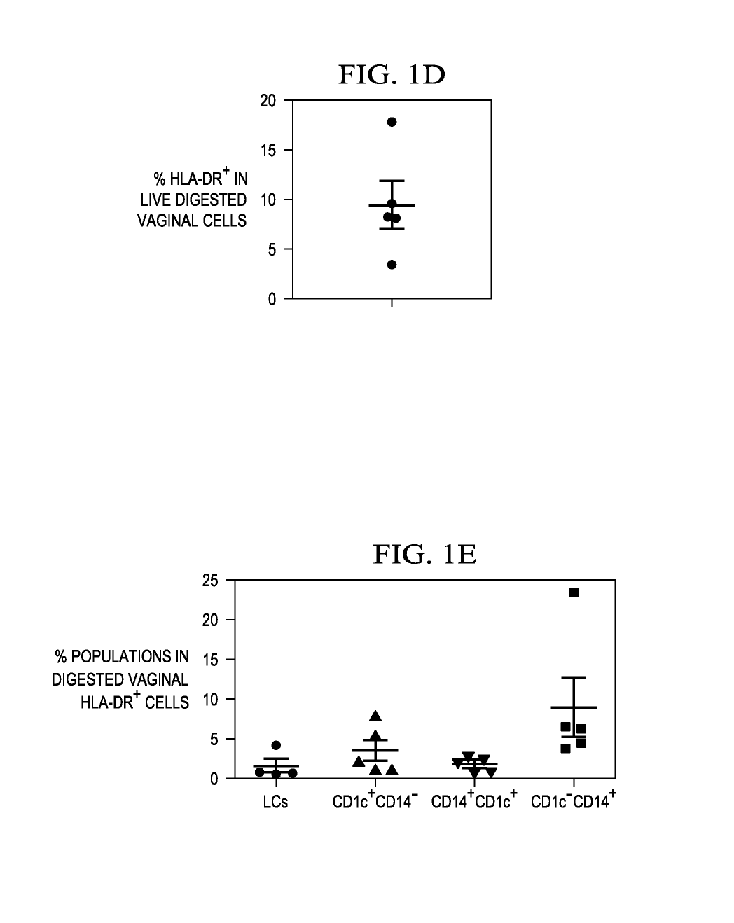

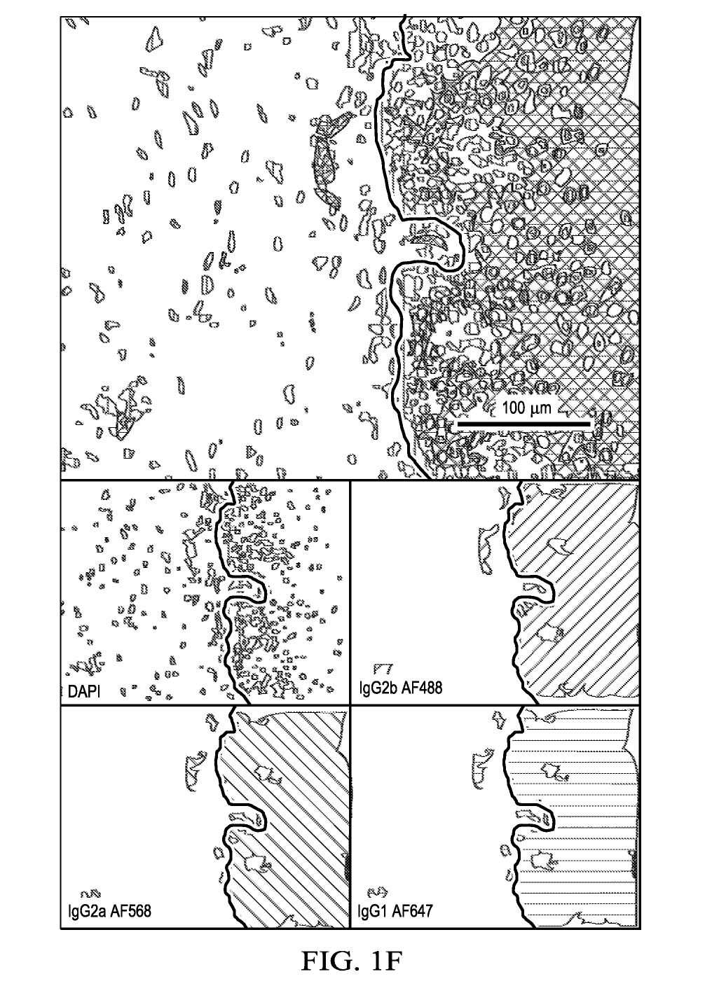

It was found that human vaginal mucosa contain four major subsets of APCs: APC subsets that localize in the human vaginal mucosa were characterized by both flow cytometry and immunofluorescence methods. Mucosal tissues were enzymatically digested and stained with 7-AAD and indicated antibodies (FIG. 1A). Live cells 7-AAD-HLA-DR⁺ cells were gated (left panel in FIG. 1A) and were separated into HLA-DR⁺angerin⁺ (CD207⁺) and HLADR⁺Langerin⁻ cells (middle panel in FIG. 1A). HLA-DR⁺langerin⁻ cells (I) were further divided into four subsets based upon CD1c and CD14 expression (right panel in FIG. 1A): CD1c⁺CD14⁻ (II), CD1c⁺CD14⁺ (III), CD1c⁻CD14⁺ (IV), and CD1c⁻CD14⁻ (V) cells. A total of five subsets of HLA-DR⁺ cells, including CD207⁺ cells, were FACS-sorted and their morphologies were examined (FIG. 1B). CD207⁺, CD1c⁺CD14⁻ and CD1c⁺CD14⁺ cells display dendrites, suggesting their classification as DCs. CD1c⁻CD14⁺ cells contain large vacuoles in the cytoplasm, which is one of the major characteristics of macrophages (MΦ). Cell morphology (FIG. 1B) as well as their expression of CD34, CD54 and CD123 (data not shown) suggest that CD1c⁻CD14⁻ cells are endothelial and/or epithelial cells.

APC subsets determined by flow cytometry were further confirmed by examining their tissue localizations with microscopy. CD207⁺ cells are mainly localized to the vaginal epithelium, while the other three subsets (CD1c⁺CD14⁻, CD1c⁺CD14⁺, and CD1c⁻CD14⁺) are in the submucosa (FIG. 1C) Tissue sections stained with isotype control antibodies are presented in FIG. 1F. The percentage of HLA-DR⁺ cells in total vaginal mucosal tissues is approximately 10% (FIG. 1D). Of the HLA-DR⁺ cells, CD1c⁻CD14⁺ cells are the most abundant APCs (FIG. 1E). The other three subsets of DCs are less than 5% of the total HLA-DR⁺ cells in the vagina. Thus, it can be concluded that human vaginal mucosa contains at least four major subsets of APCs, CD207⁺, CD1c⁺CD14⁻, CD1c⁺CD14⁺, and CD14⁺CD1c⁻. The inventors were also able to detect BDCA2⁺ plasmacytoid DCs (pDCs) and CD19⁺ B cells, but, in general, the frequency of pDCs (0.09±0.1% of total cells) and B cells (0.2±0.3% of total cells) was low. In three out of four donors tested, BDCA3 expression was measurable. However, only 0.19±0.18% of the total vaginal cells were BDCA3⁺, and more than 65% of BDCA3⁺ cells were also CD1c⁺. Thus, the data presented herein suggests that pDCs and the recently described BDCA3⁺ Clec9A⁺CD1c− DCs (Jongbloed et al., 2010; Poulin et al., 2010) are present, but at low levels in human vaginal tissues examined herein.

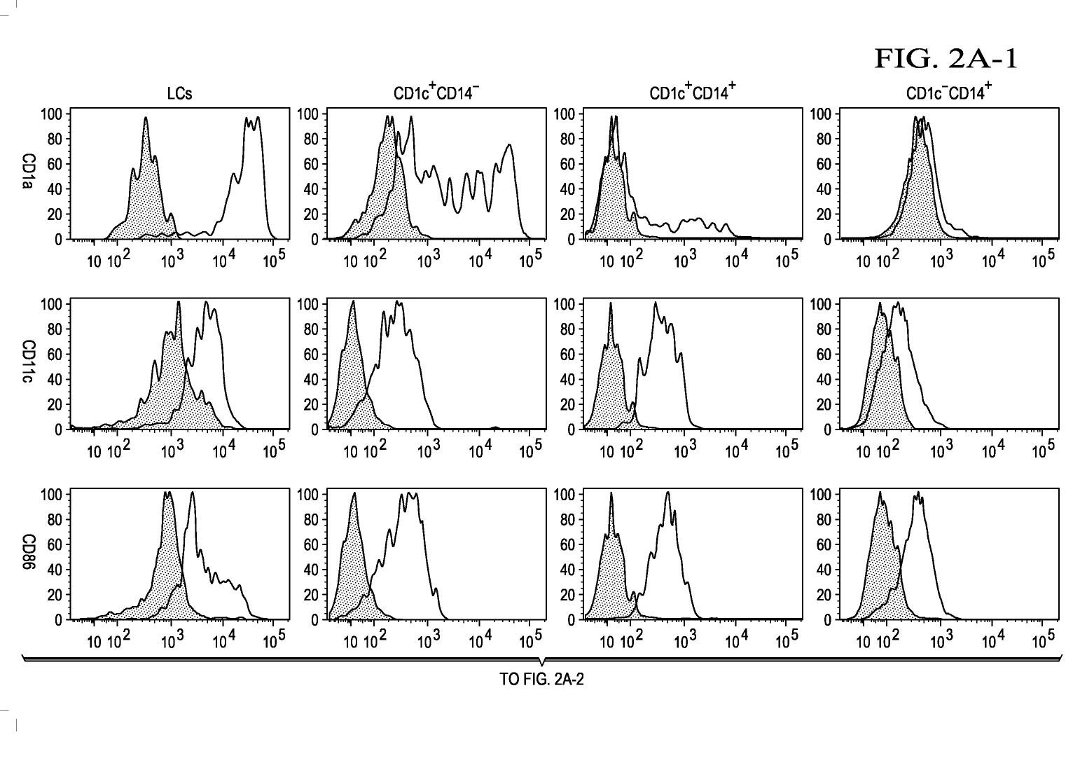

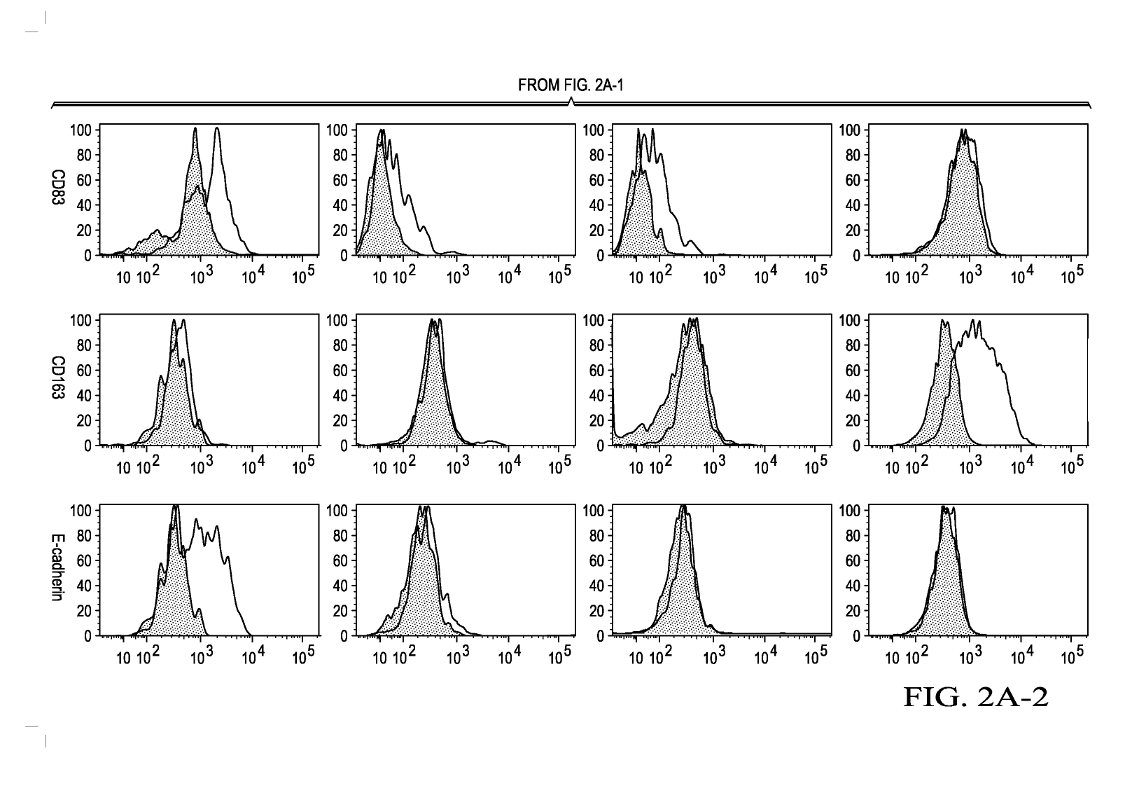

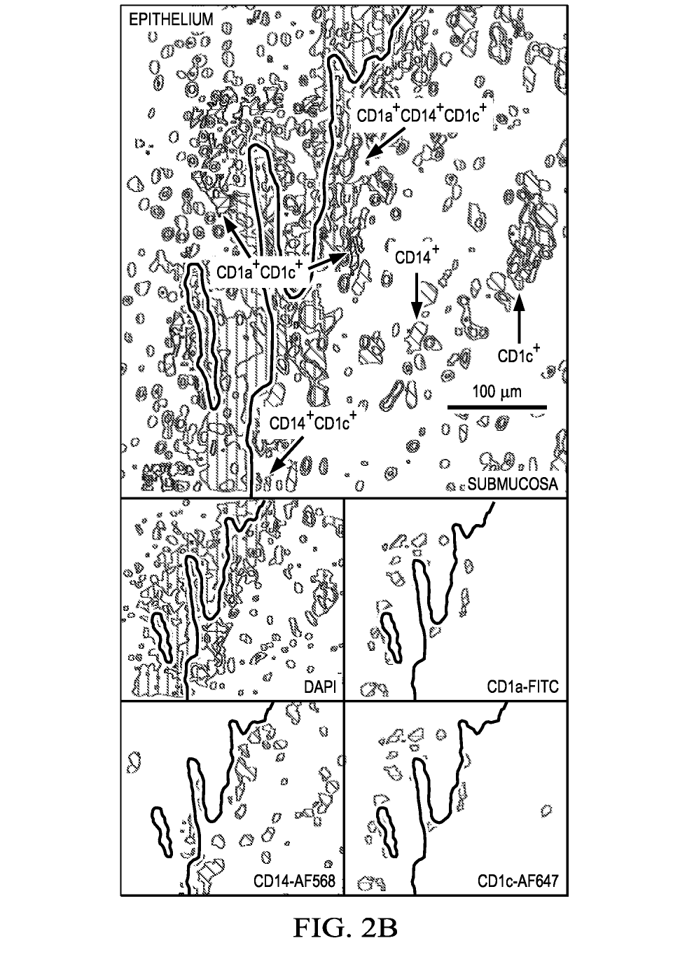

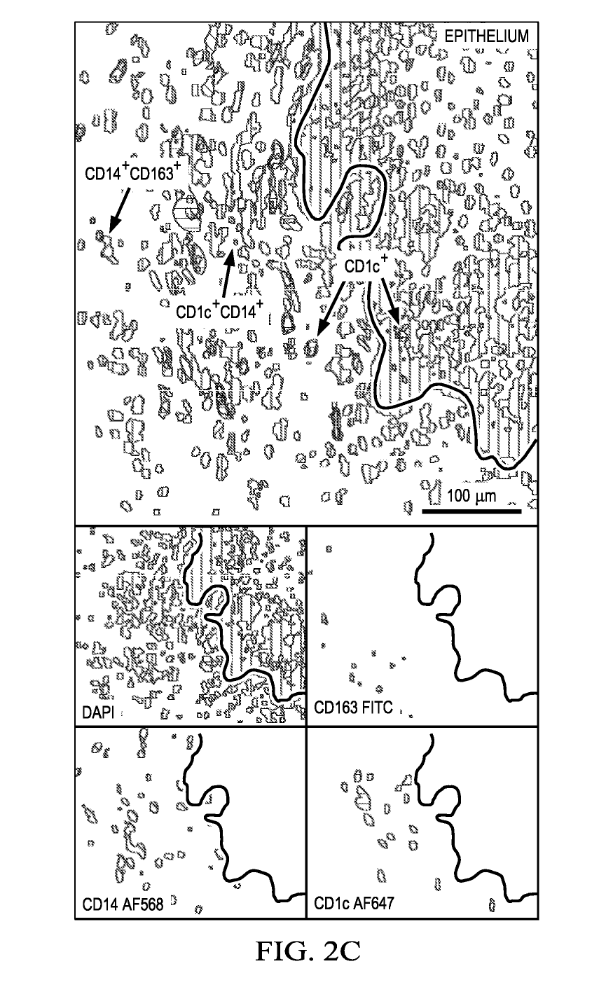

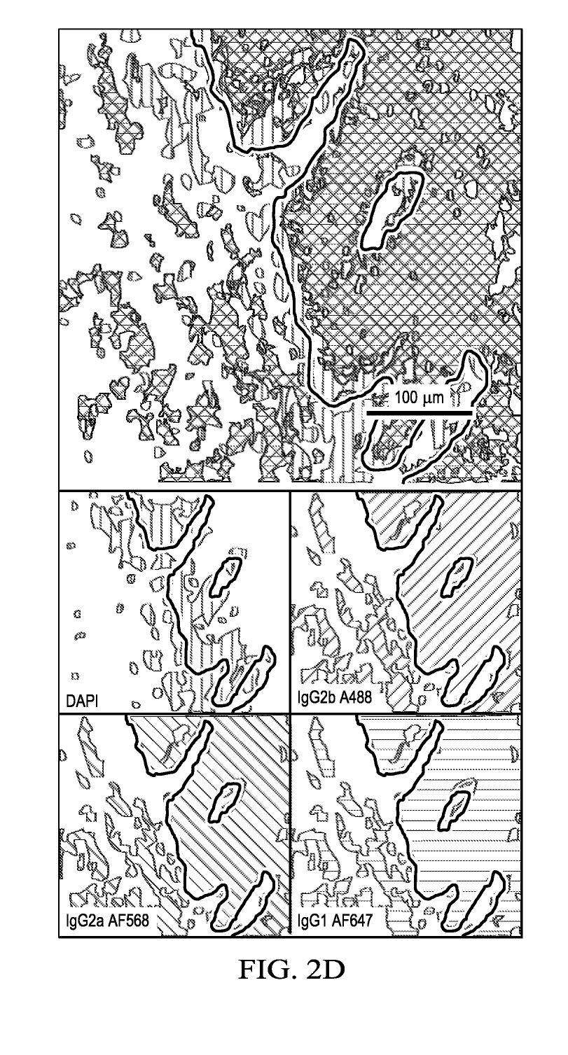

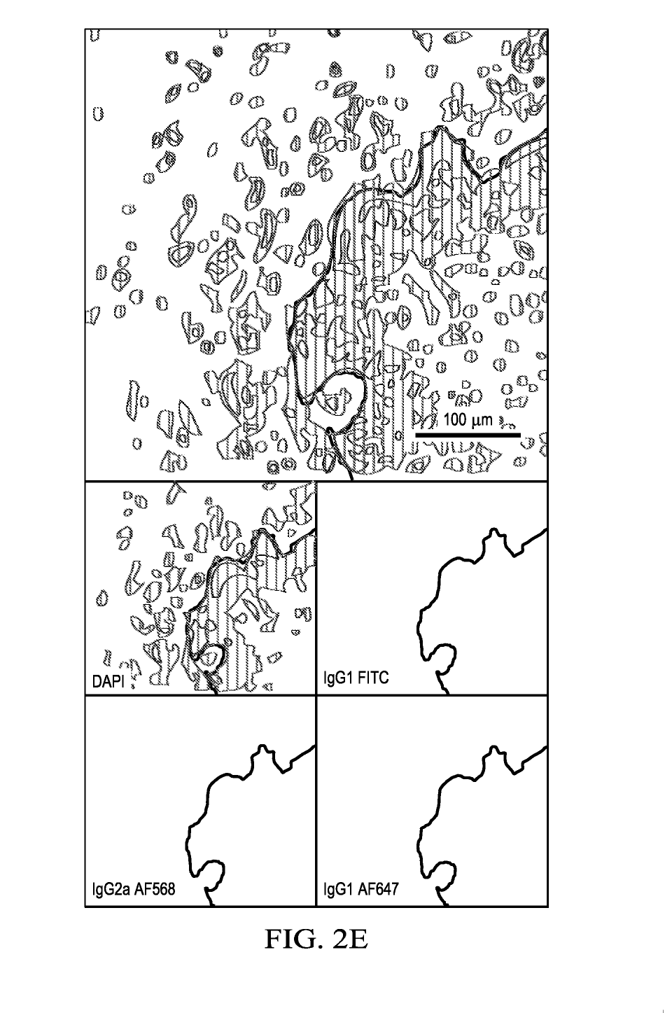

Phenotypes of Vaginal APC Subsets: The phenotypes of the four vaginal APC subsets were further characterized by examining the expression of other surface molecules (FIG. 2A). The vaginal CD207⁺ cells express both CD1a and E-cadherin, which are known to be expressed on the surface of LCs (Blauvelt and Katz, 1995). LCs, as well as the CD1c⁺CD14⁺ and CD1c⁺CD14− DC subsets, express both CD86 and CD83. CD1c⁻CD14⁺ cells express CD86, but not CD83. In addition, CD1c-CD14⁺ cells express CD163, which is known to be expressed on macorphages (Zaba et al., 2007). Compared to LCs, CD1c⁺CD14⁺ and CD1c⁺CD14⁻ DCs, CD1c-CD14⁺ cells express lower levels of CD11c, which is in accordance with the classification of these cells as macrophages.

Expression of CD1a and CD163 was further confirmed by examining APC subsets localized in the vaginal tissues (FIG. 2B). CD1c⁺CD1a⁺ DCs are localized in both epithelium and submucosa. The present inventors were also able to detect CD1a⁺CD1c⁺CD14⁺ cells, but not CD1a⁺CD1c-CD14⁺, in the submucosa. CD1c⁻CD14⁺ macrophages were mainly localized in the submucosa and they express CD163 (FIG. 2C). None of the CD1c⁺CD14⁻ DCs or CD1c⁺CD14⁺ DCs express CD163. The inventors also observed that CD1c⁺CD14⁻ DCs and CD1c⁺CD14⁺ submucosal DCs were mainly localized to the proximal site of the epithelium, whereas CD1c⁻CD14⁺ macrophages were found throughout the submucosa. Isotype control stainings for FIGS. 2B and 2C are presented in FIGS. 2D and 2E, respectively.

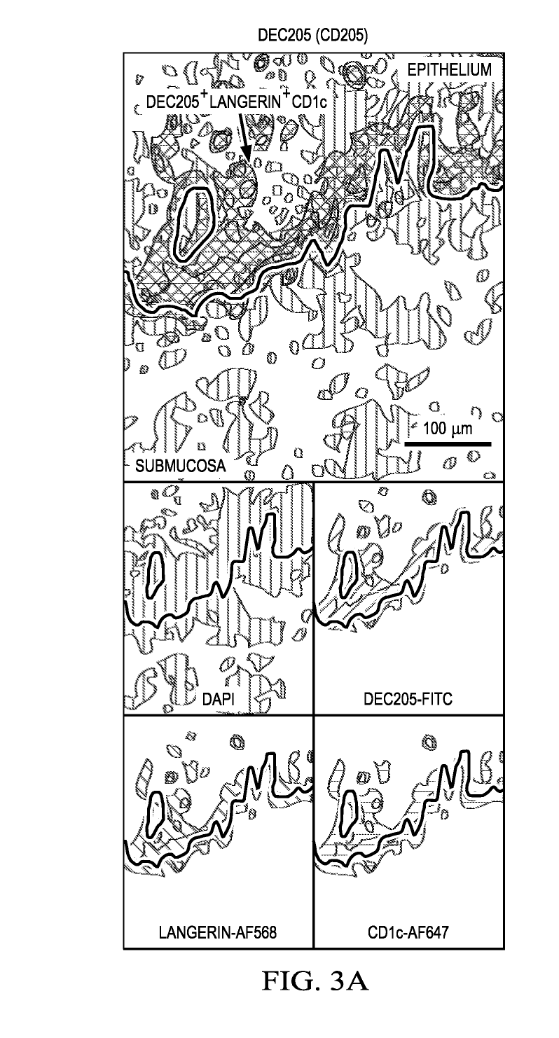

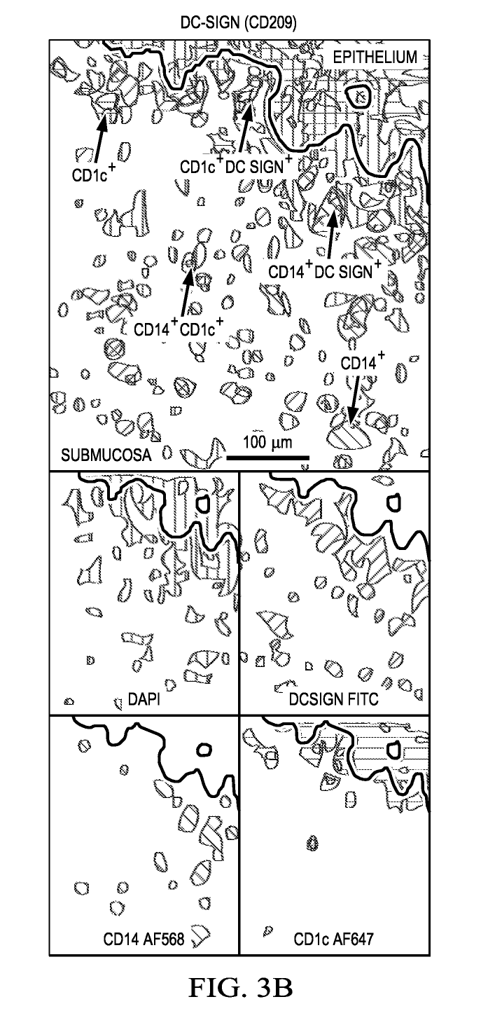

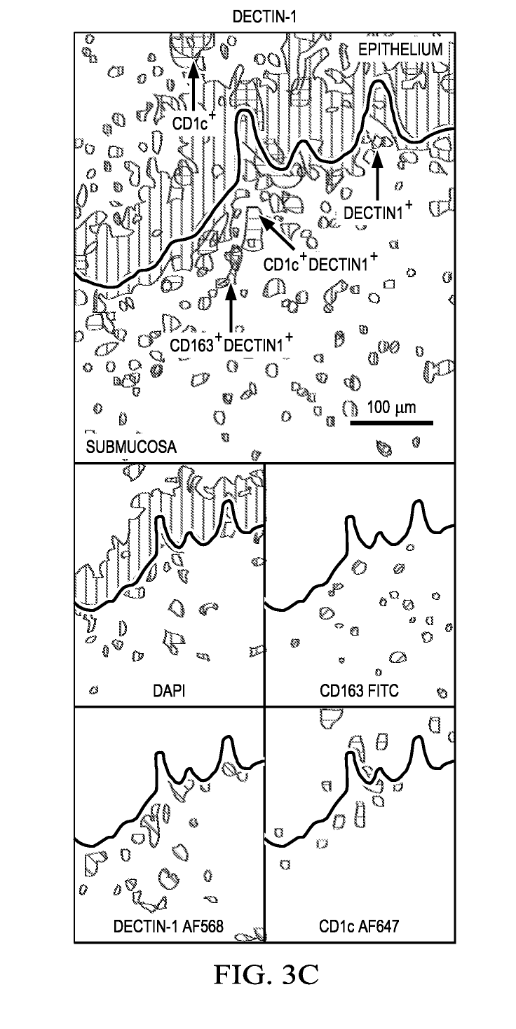

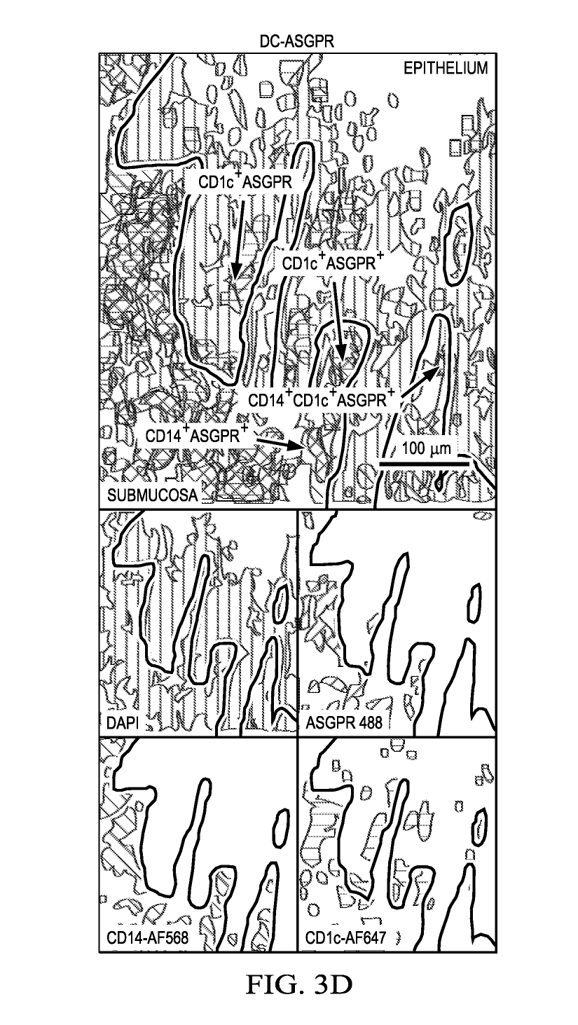

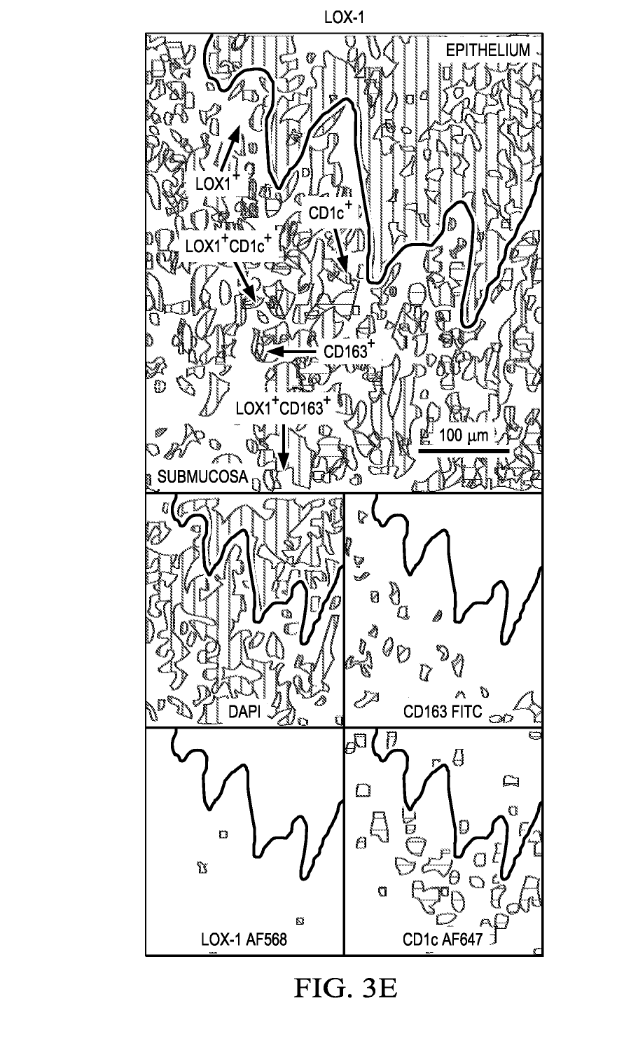

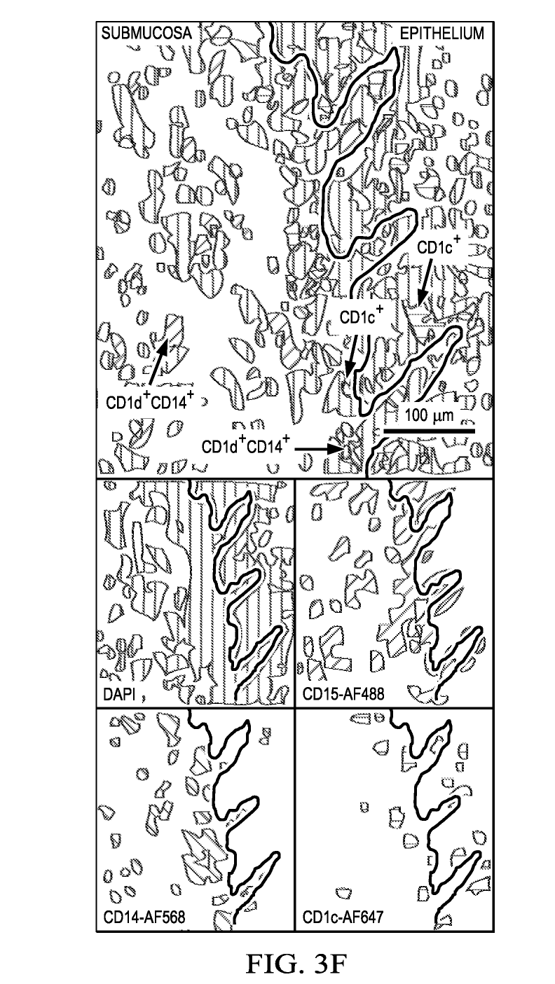

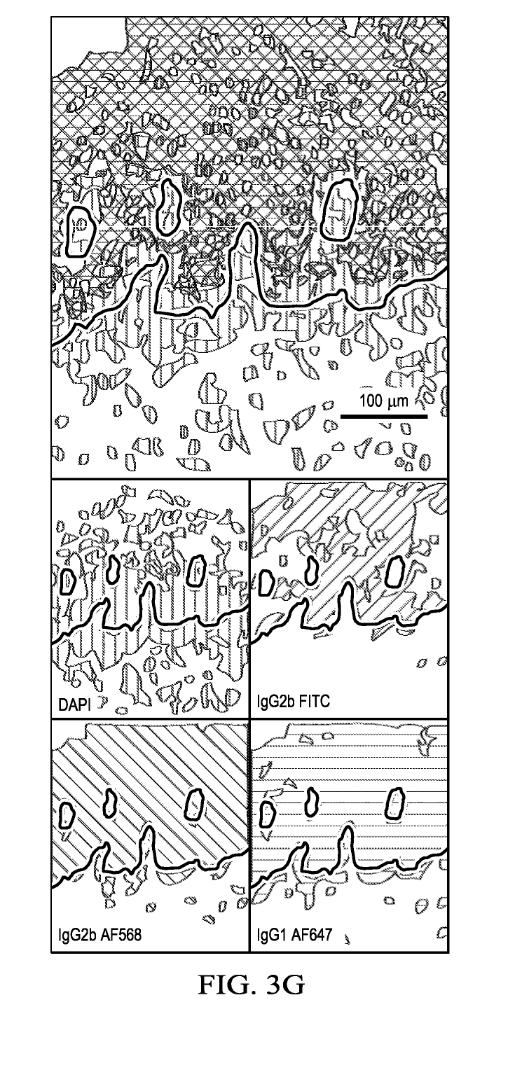

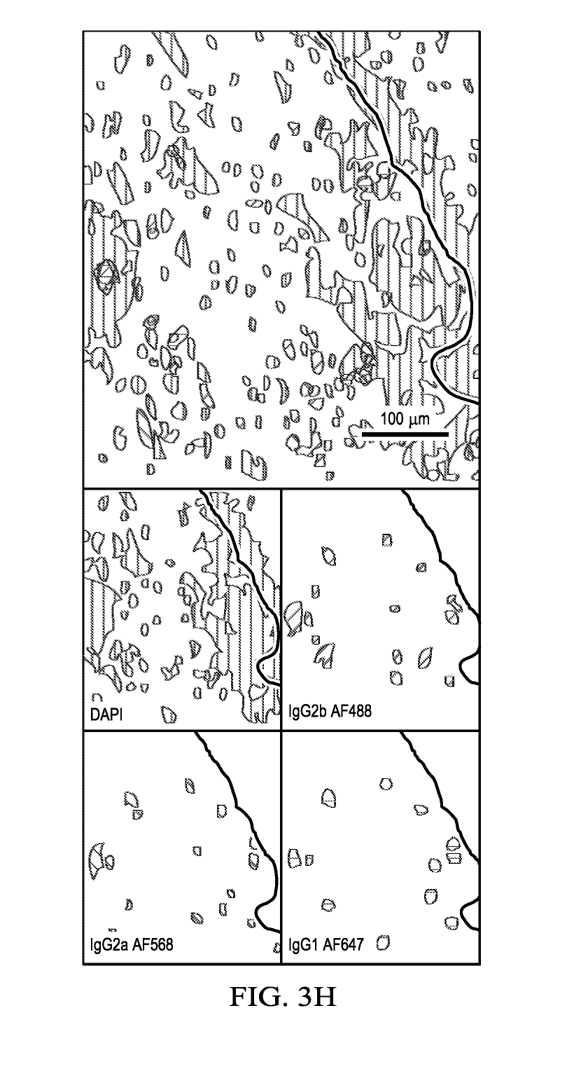

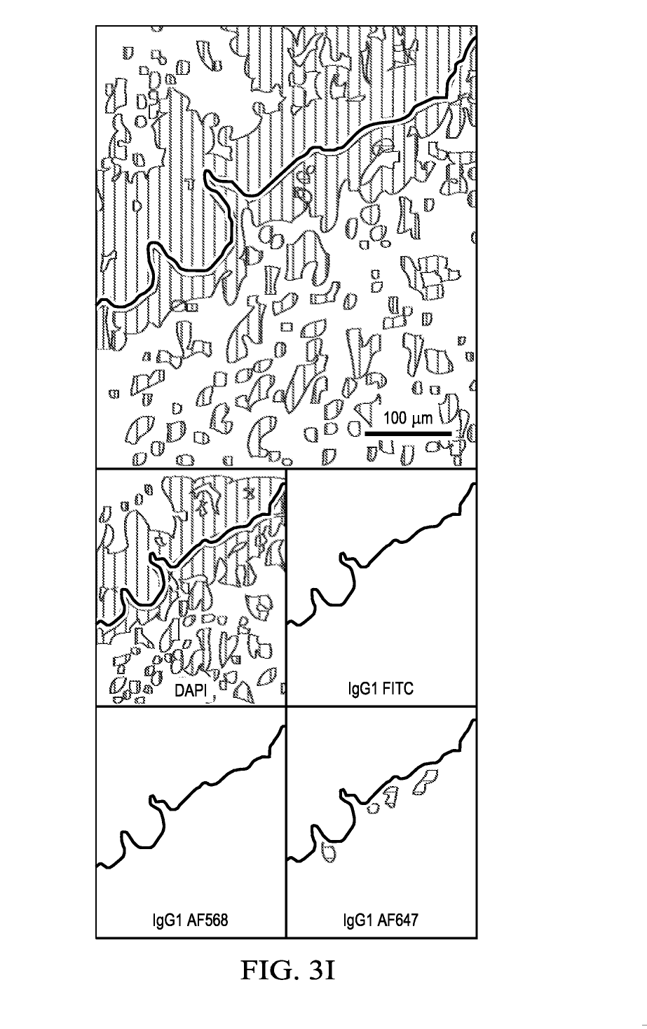

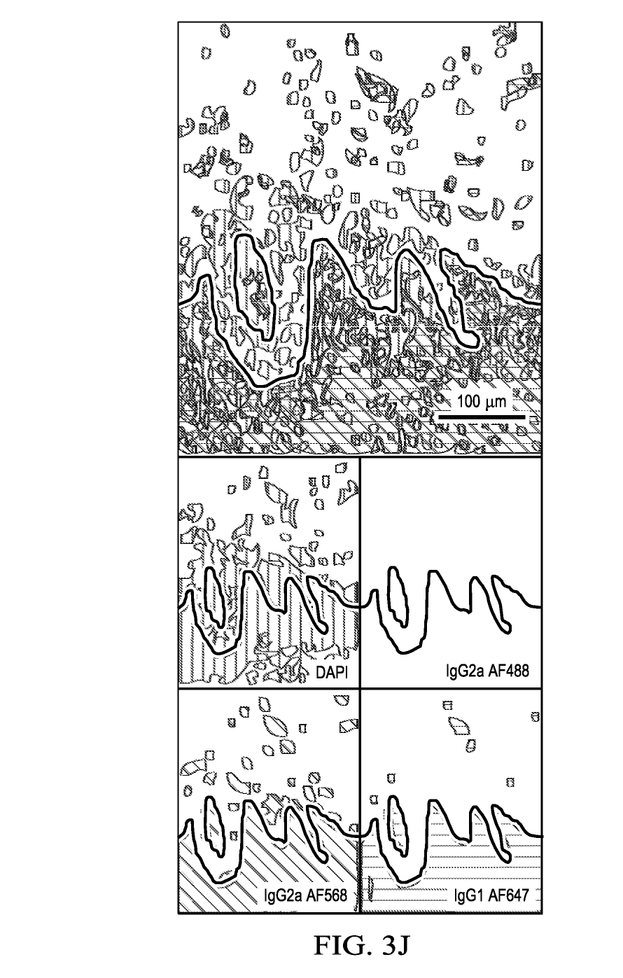

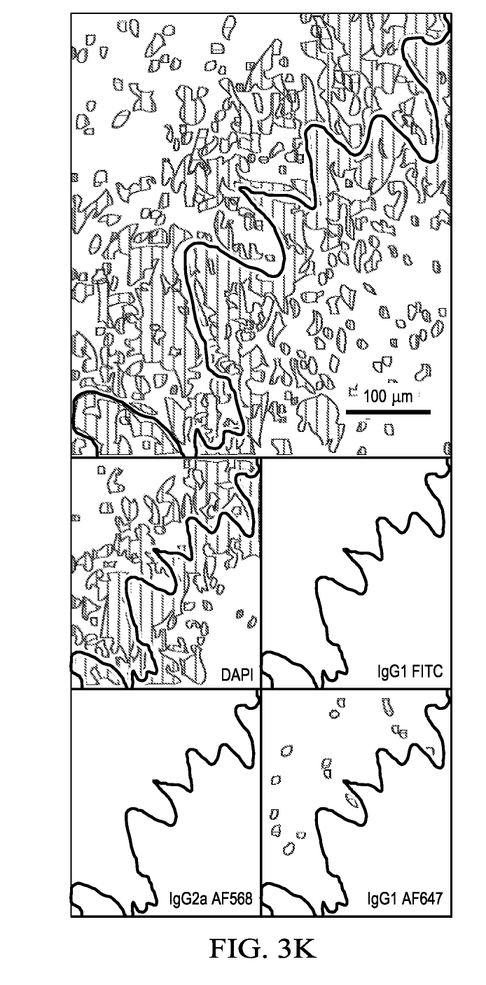

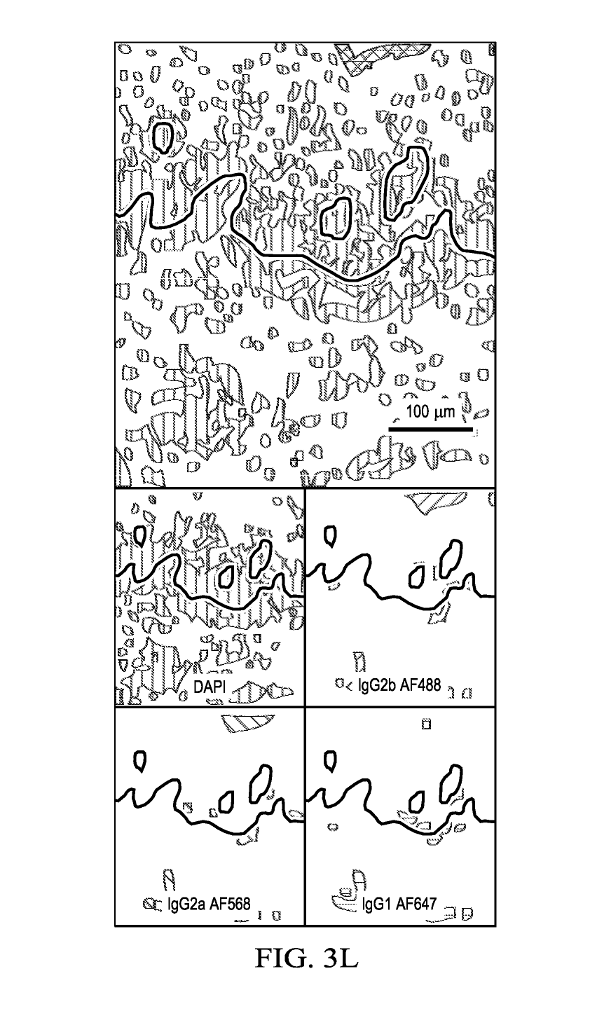

Lectin-Like Receptor (LLR) and CD1d Expression in Vaginal APC Subsets: Different subsets of DCs express distinct patterns of LLRs (Dudziak et al., 2007; Poulin et al., 2010; Shortman and Liu, 2002; Soares et al., 2007). Furthermore, individual LLRs possess common and distinct functions (Brown, 2006; Delneste et al., 2002; Figdor et al., 2002; Geijtenbeek and Gringhuis, 2009) in regulating immune responses. In addition, CD1d is highly associated with host immune responses to HSV infections (Yuan et al., 2006). Thus, the inventors tested whether the four subsets of vaginal APCs express DEC205 (CD205), DC-specific ICAM-3 grabbing non-integrin (DC-SIGN: CD209), Dectin-1, DC-asialoglycoprotein receptor (DC-ASGPR), lectin-like oxidized low-density lipoprotein receptor-1 (LOX-1) and CD1d. FIG. 3A shows that CD205 is mainly expressed in LCs localized in the epithelium. The majority of CD1c-CD14⁺ macrophages express CD209 (FIG. 3B), which is associated with HIV transmission (Geijtenbeek et al., 2000; Jameson et al., 2002). Only a part of CD1c⁺ submucosal DCs express CD209. In contrast to CD205 and CD209, Dectin-1 expression is widely distributed on both macrophages and submucosal DCs (FIG. 3C). Dectin-1 was not detected in LCs, but it was expressed in other cell types that do not express either CD163 or CD11c in the submucosa. Both DC-ASGPR and LOX-1 are scavenger receptors that contain an ITAM-like motif (Delneste et al., 2002; Valladeau et al., 2001) and thus can participate in host immune responses by taking up antigens and delivering intracellular signals to activate DCs. FIG. 3D shows that DC-ASGPR is expressed by both CD1c⁺CD14− and CD1c⁺CD14⁺ submucosal DCs and by CD1c-CD14⁺ macrophages. Not all, but some CD1c⁺ DCs in epithelium express DC-ASGPR. LOX-1 is expressed in CD163⁺ macrophages and some of CD1c⁺ submucosal DCs (FIG. 3E). Fractions of other cell types in submucosa that are CD1c− and CD163− also express LOX-1. The majority of CD14⁺ cells express CD1d, but CD1c⁺ submucosal DCs and LCs in the epithelium express low or undetectable levels of CDid (FIG. 3F). Isotype control stainings for the data in FIGS. 3A-3F are presented in FIGS. 3G-3L. Taken together, the data presented hereinabove demonstrates that individual APC subsets localized in the human vagina express distinct patterns of LLRs and CD molecules.

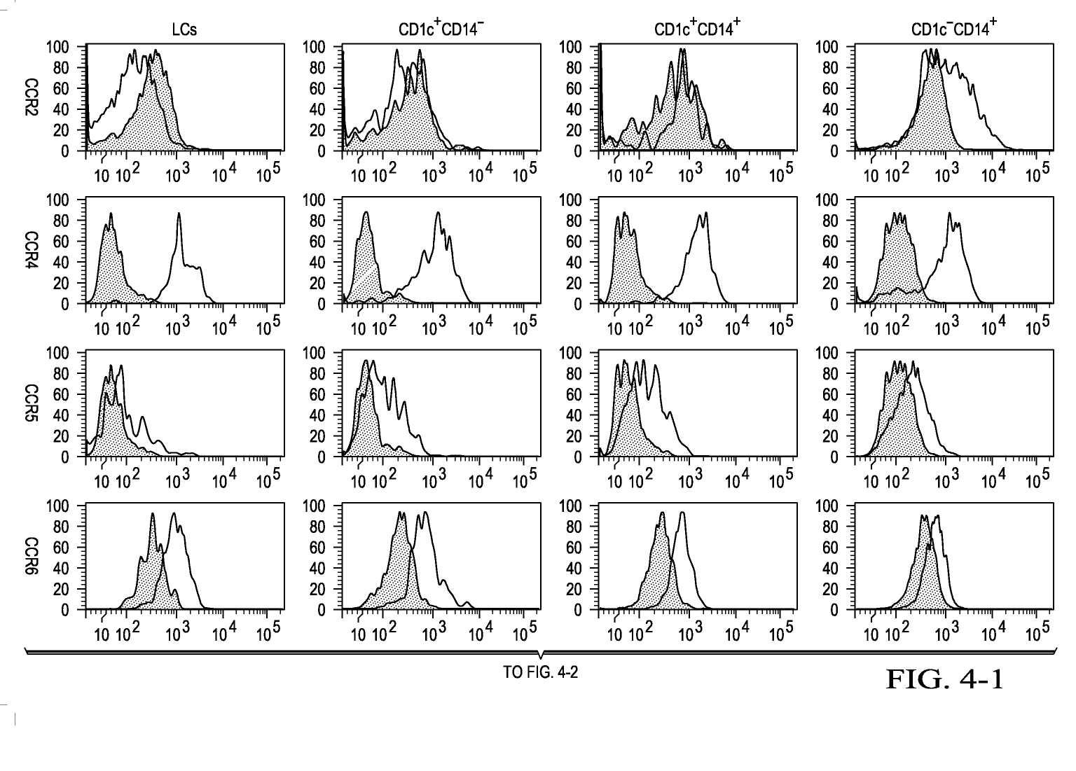

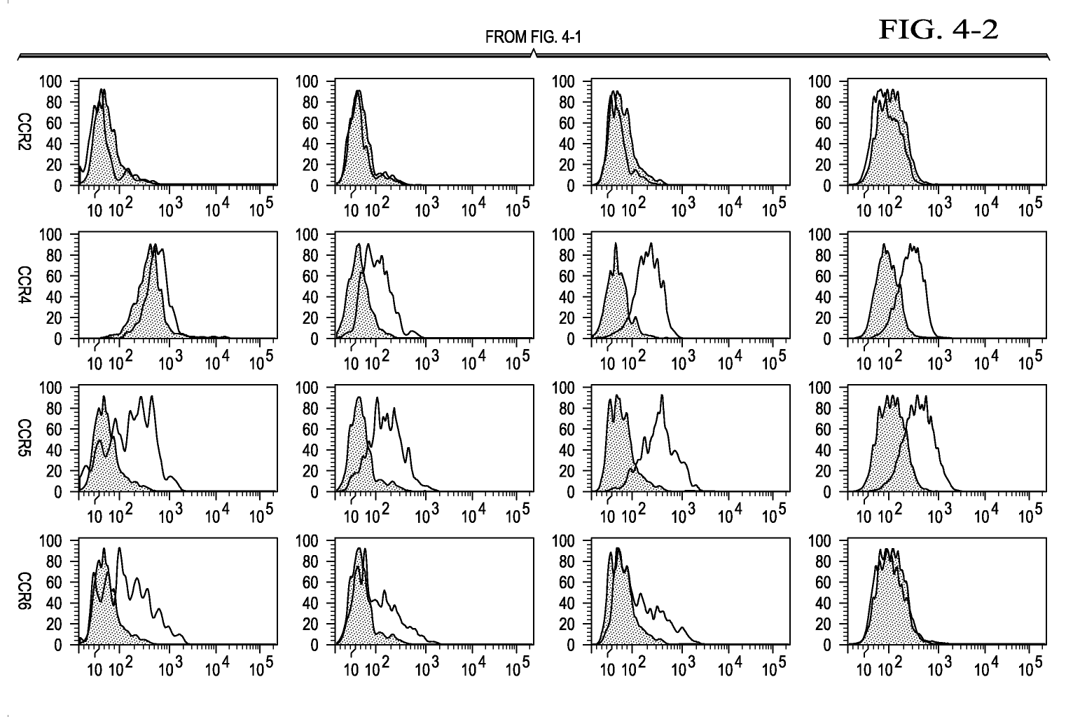

Chemokine Receptor and β7 Integrin Expression on Vaginal APC Subsets: The inventors also tested whether different subsets of vaginal APCs express distinct patterns of chemokine receptors and β7 integrin (FIG. 4). CCR2, a homing receptor for monocytes andmacrophages (Serbina et al., 2008), is detected only on the surface of CD1c-CD14⁺ macrophages, but not DC subsets. As previously reported (Hladik et al., 2007), CCR5 and CXCR4 are expressed on LCs as well as on the other three subsets of submucosal APCs in the vagina, even though the level of CXCR4 is minimal (FIG. 4). Compared to LCs, submucosal DCs express higher levels of both CCR5 and CXCR4. Both LCs and submucosal DCs exhibit similar surface expression levels of CCR6, a receptor found on intestinal DCs (Williams, 2004). CCR6 was also detected on the surface of CD1c-CD14⁺ macrophages, but the expression level was lower than those on the vaginal DC subsets. Moreover, LCs and submucosal DCs express β7 integrin, but CD1c-CD14⁺ macrophages did not. Both CCR4 and CX3CR1 were equally expressed on the surface of the four vaginal APC subsets. CCR7 was not detected on the surface of the vaginal APC subset. Taken together, the data demonstrates that subsets of human vaginal APCs express common as well as distinct patterns of chemokine receptors and β7 integrin.

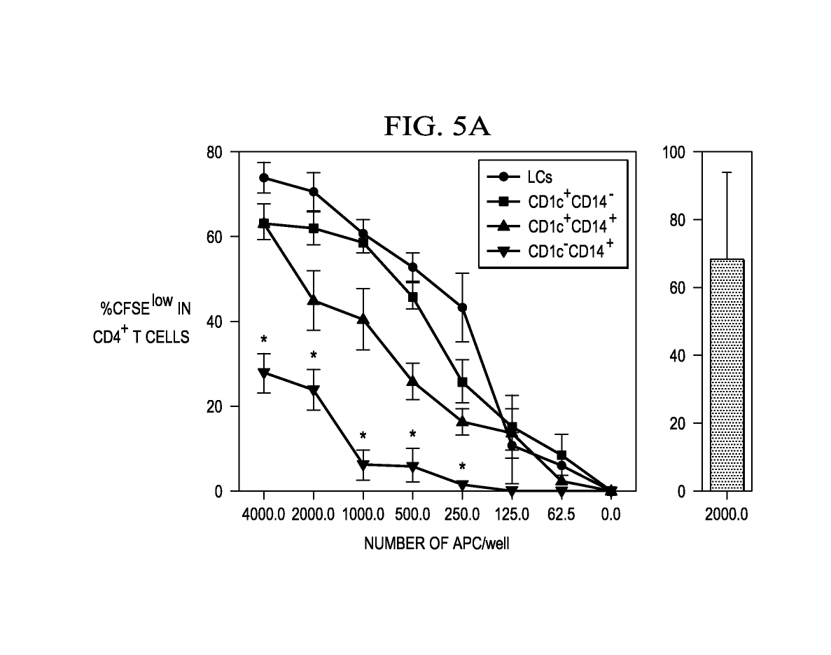

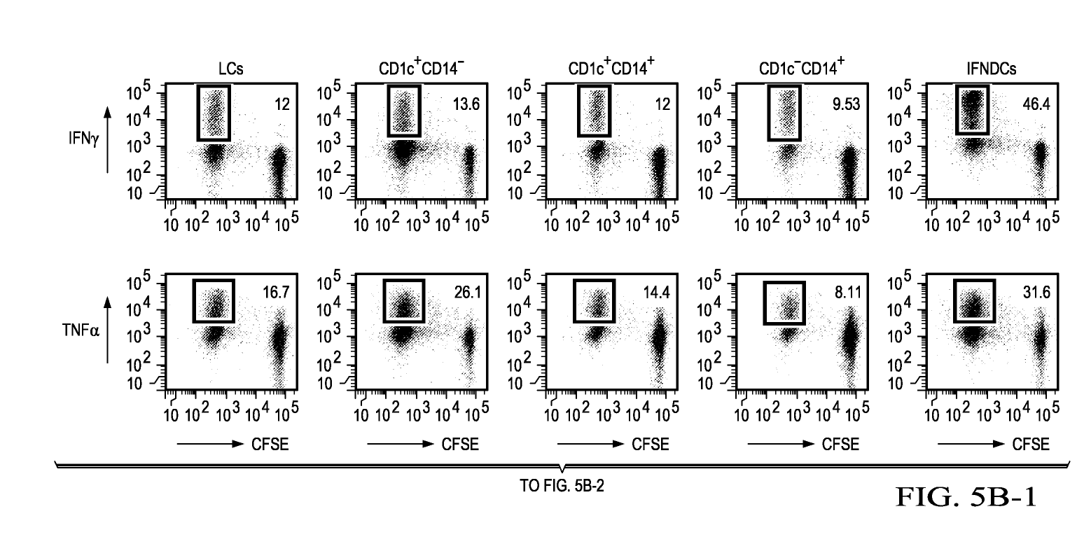

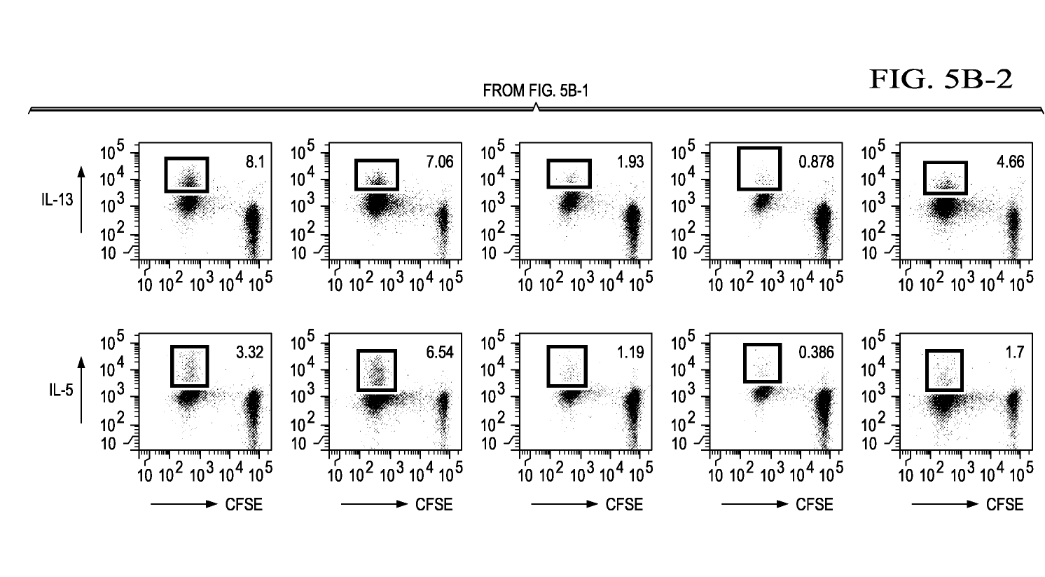

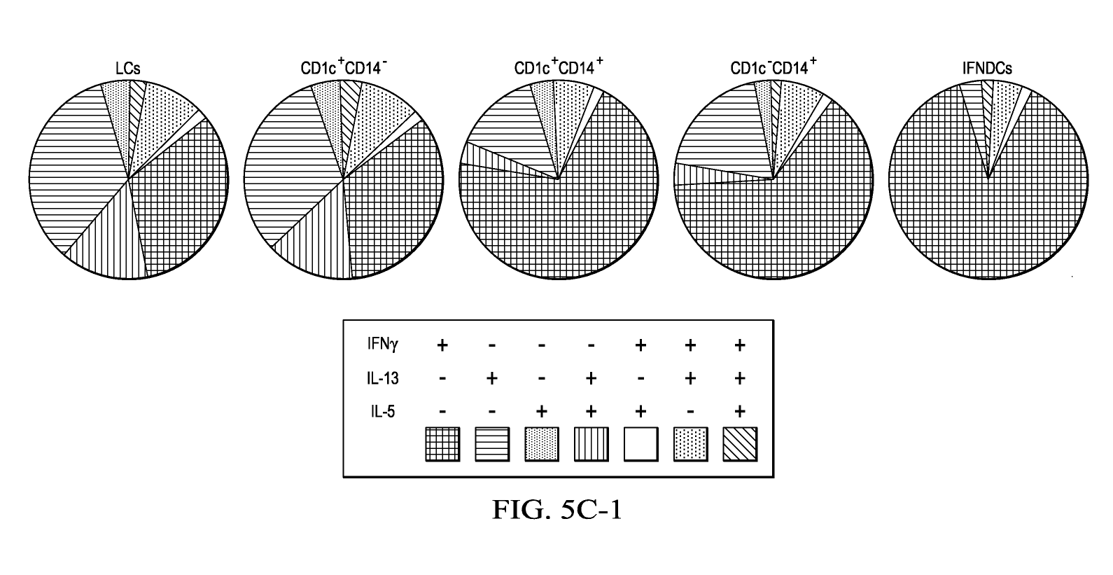

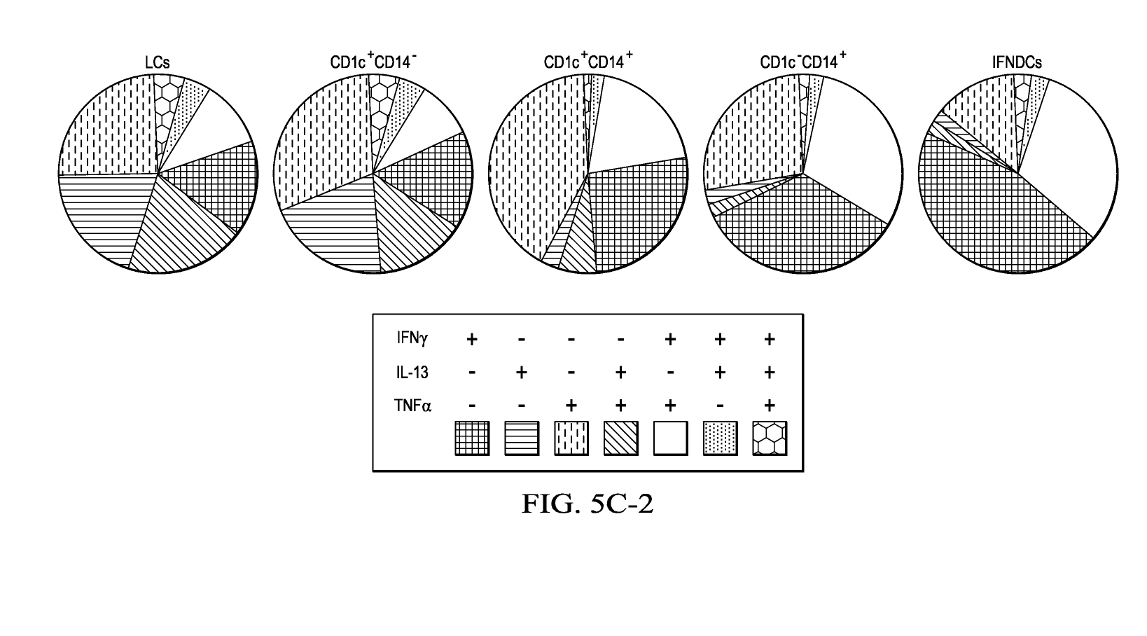

Polarization of Naïve Cd4⁺ T Cell Responses by Vaginal APC Subsets: To test the capacity of vaginal APC subsets in eliciting CD4⁺ T cell responses, the inventors first assessed proliferation of allogeneic naïve CD4⁺ T cells induced by the four subsets of APCs (left panel in FIG. 5A). CFSE-labeled T cells were co-cultured with increasing numbers of FACS-sorted APC subsets for seven days. CD4⁺ T cell proliferation was assessed by measuring CFSE dilution. Three subsets of vaginal DCs induced greater CD4⁺ T cell proliferation than did CD1c-CD14⁺ macrophages. However, LCs and CD1c⁺CD14⁻ DCs induced greater naïve CD4⁺ T cell proliferation than did CD1c⁺CD14⁺ DCs. In fact, 60.5±6.2%, 58.5±5.5%, 40.5±14% and 6.2±7% of naïve CD4⁺ T cells had undergone proliferation in the presence of 1×10³ LCs, CD1c⁺CD14+ DCs, CD1c⁺CD14⁺ DCs and CD1c-CD14⁺ macrophages, respectively (ratio 1:100; p<0.01 for LCs vs. CD1c⁺CD14⁺ DCs, p<0.05 for CD1c⁺ DCs vs. CD1c⁺CD14⁺ DCs and p<0.001 for each DC subset compare to CD1c-CD14⁺ macrophages). Monocyte-derived in vitro-cultured IFNDCs, a mixed population of DCs expressing Langerin, CD1c and/or CD14 (data not shown), were used as controls. The levels of CD4⁺ T cell proliferation induced by IFNDCs (2×10³) were similar to what was induced by LCs or CD1c⁺CD14⁻ DCs (right panel in FIG. 5B).

The inventors then assessed the quality of CD4⁺ T cells by measuring the percentage of CD4⁺ T cells expressing IFNγ, TNFα, IL-13, and IL-5. At day 7 after the co-cultures of allogeneic T cells with individual APC subsets, CD4⁺ T cells were stimulated with phorbol-12-myristate 13-acetate (PMA) and ionomycin in the presence of brefeldin A (BFA). Live CD4⁺ T cells were gated and the percentages of cytokine-expressing CD4⁺ T cells were measured (FIG. 5B). All four subsets of vaginal APCs induced similar percentages of IFNγ⁺CD4⁺ T cells, ranging from 8.7±6% to 14.7±7% of total CD4⁺ T cells (FIGS. 5B and 5D). The percentages of TNFα⁺CD4⁺ T cells induced by LCs (25±8.6%) and CD1c⁺CD14− DCs (32.7±4.2) were comparable, but greater than the ones induced by CD1c⁺CD14⁺ DCs (10.3±3.5%) and CD1c-CD14⁺ macrophages (9.2±6.2%). Interestingly, LCs and CD1c⁺CD14− DCs resulted in greater numbers of CD4⁺ T cells expressing Th2-type cytokines, IL-13 and IL-5. Summarized data from six independent studies are presented in FIG. 5D. IL-21⁺CD4⁺ T cells were similarly induced by all APC subsets (≈7.3±4.6%), while IL-10⁺CD4⁺ T cells were undetectable (data not shown). Overall, the percentage of IL-17⁺ cells induced by each subset was below 0.1% of total CD4⁺ T cells (0.06±0.04, 0.08±0.03, 0.08±0.1 and 0.04±0.02 for LCs, CD1c⁺CD14⁻DCs, CD1c⁺CD14⁺ DCs and CD1c-CD14⁺ macrophages, respectively, from six independent studies).

To further analyze the quality of CD4⁺ T cells induced by different subsets of vaginal APCs, the inventors applied a Boolean gating strategy. As shown in FIG. 5C (left panels), LCs and CD1c⁺CD14⁻ DCs were able to polarize naïve CD4⁺ T cells toward Th2-type, whereas CD1c⁺CD14⁺ DCs and CD1c⁻CD14⁺ macrophages polarized them toward Th1-type. LC-induced CD4⁺ T cells express IL-5 (1%), IL-13 (10%), or both IL-5 and IL-13 (4%). Some of Th2-type CD4⁺ T cells that are induced by LCs express IFNγ (4%). Only 10% of LC-induced CD4⁺ T cells were IFNγ single-positive cells. The capacity of LCs and CD1c⁺CD14⁻ DCs to induce Th2-type responses was further confirmed by measuring cytokines secreted from CFSE^{low} CD4⁺ T cells (FIG. 5E). After 7 days of the co-culture of CFSE-labeled naïve T cells and APC subsets (IFNDCs, LCs, or CD1c⁺CD14⁻ DCs), CFSE^{low}CD4⁺ T cells were FACS-sorted. They were then stimulated with anti-CD3 and anti-CD28 antibodies for 48 h, and the amounts of cytokines (IFNγ, IL-5, and IL-13) in culture supernatants were assessed. Consistent with the data in FIGS. 5A-5D, CD4⁺ T cells induced with LCs and CD1c⁺CD14⁻ DCs secreted greater amounts of both IL-5 and IL-13, but less IFNγ, than CD4⁺ T cells induced with IFNDCs. It is also of note that CD4⁺ T cells induced with LCs secrete greater amounts of both IL-5 and IL-13 than those induced with CD1c⁺CD14⁻ DCs. In contrast to LCs and CD1c⁺CD14⁻ DCs, CD1c⁺CD14⁺ DCs and CD1c⁻CD14⁺ macrophages were able to polarize naïve CD4⁺ T cells mainly toward Th1-type (left panel in FIG. 5C). Interestingly, the majority of IFNγ⁺ CD4⁺ T cells induced with either CD1c⁺CD14⁺ DCs or CD1c-CD14⁺ macrophages do not express IL-5 or IL-13. The majority of TNFα⁺CD4⁺ T cells induced by both CD1c⁺CD14⁺ DCs and CD1c⁻CD14⁺ macrophages were also TNFα single-positive, while the cells induced by LCs and CD1c⁺CD14⁻ DCs also expressed IFNγ, IL-13, or both IFNγ and IL-13 (right panels in FIG. 5C). Taken together, it can be concluded that LCs and submucosal DCs, specifically CD1c⁺CD14⁻ DCs, are more potent than CD1c⁻CD14⁺ macrophages for inducing naïve CD4⁺ T cell proliferation. Furthermore, LCs and submucosal CD1c⁺CD14⁻ DCs polarize naïve CD4⁺ T cells mainly towards Th2-type, whereas CD1c⁺CD14⁺ DCs and CD1c⁻CD14⁺ macrophages polarize them toward Th1-type.

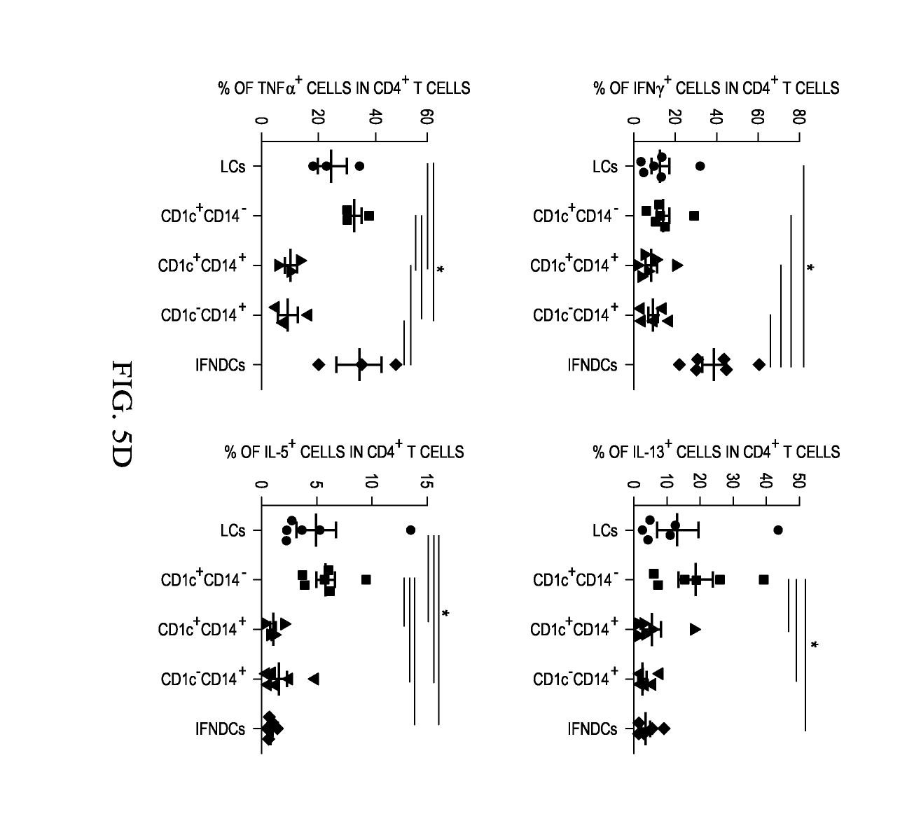

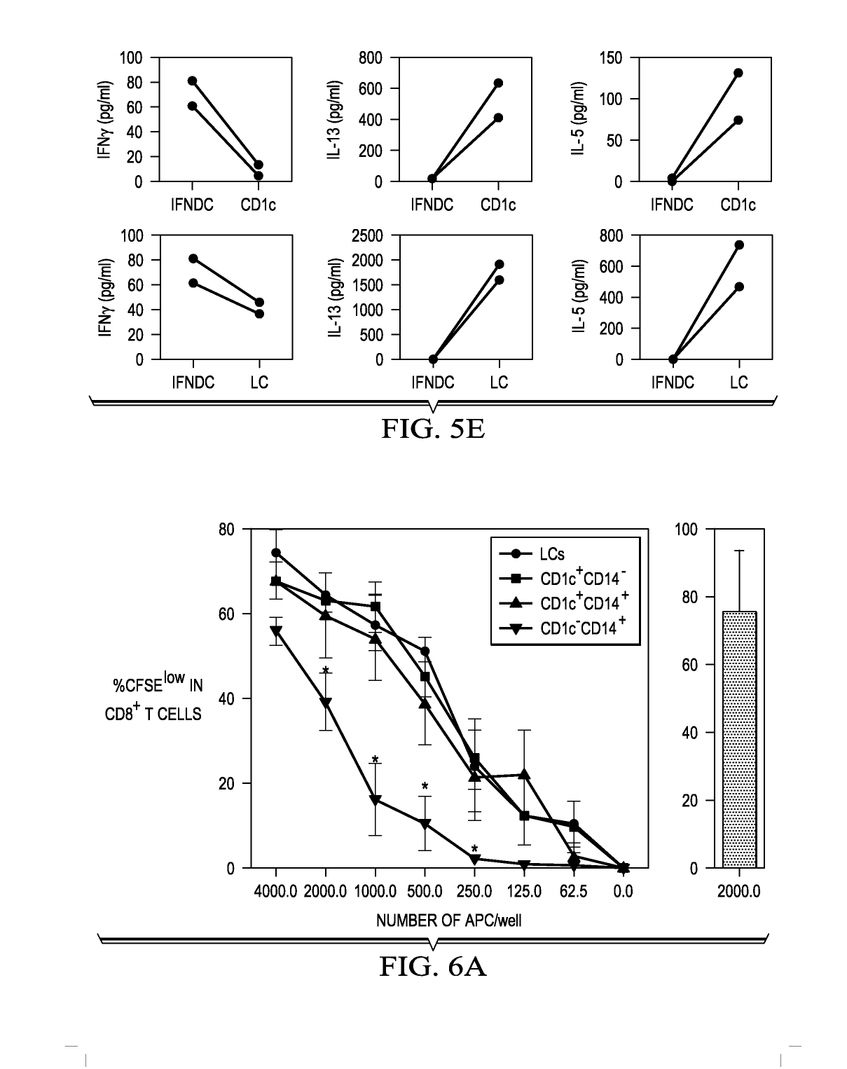

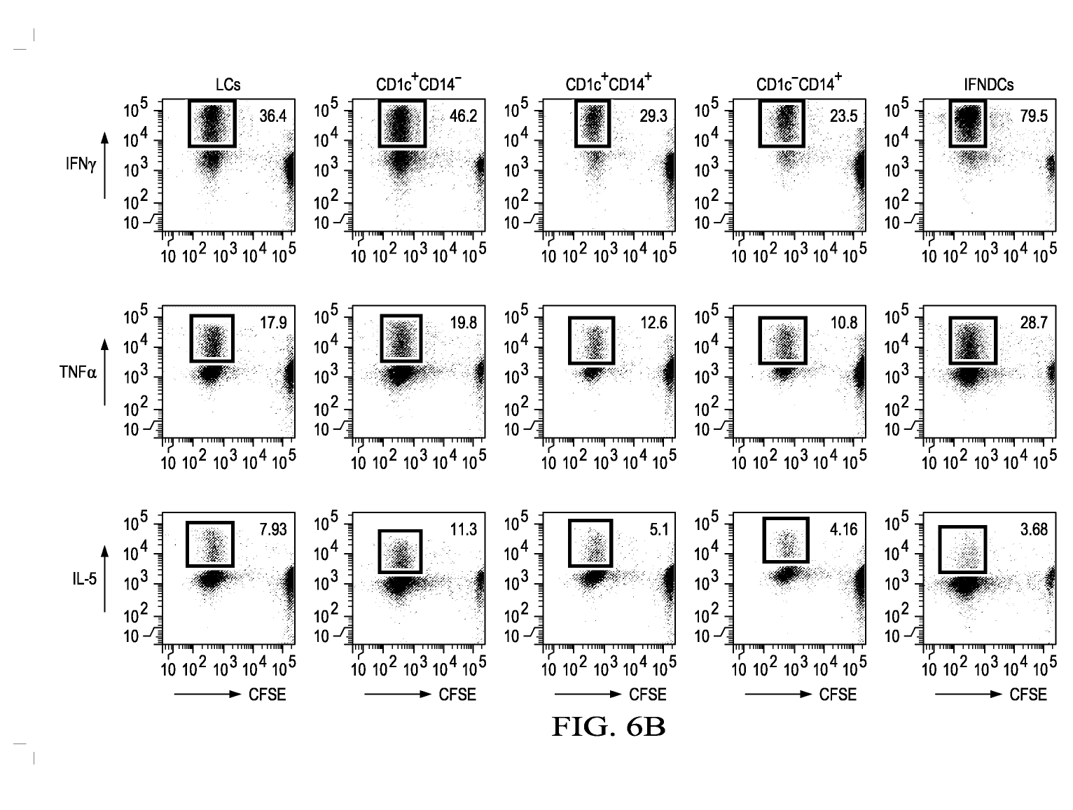

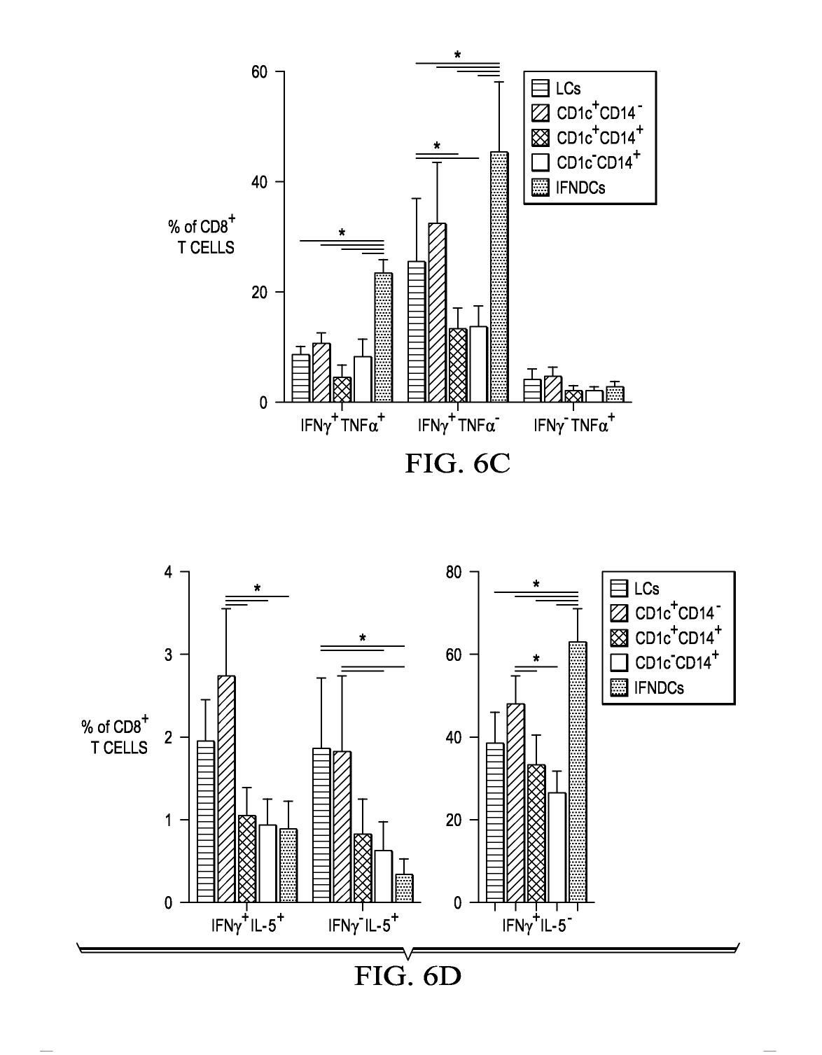

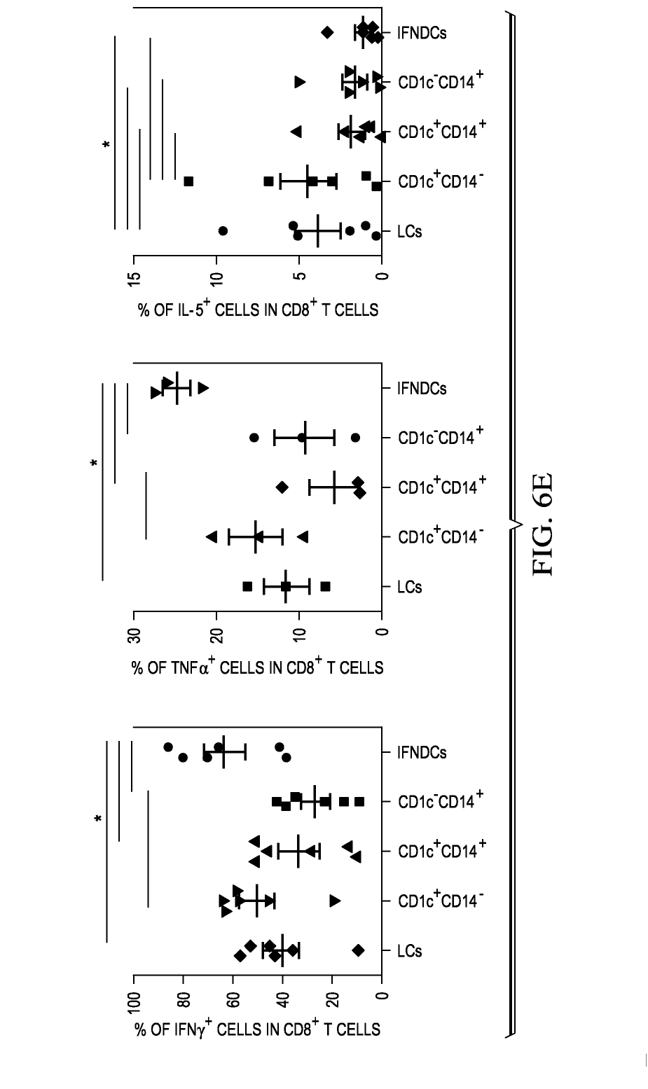

Subsets of vaginal APCs display common and distinct functions in eliciting CD8⁺ T cell responses. The inventors compared the capacity of vaginal APC subsets for inducing naïve CD8⁺ T cell responses by measuring proliferation and intracellular cytokine expression in CD8⁺ T cells. Compared to CD1c⁻CD14⁺ macrophages, LCs and submucosal DCs resulted in enhanced CD8⁺ T cell proliferation (FIG. 6A). Although LCs and CD1c⁺CD14⁻ DCs induced greater naïve CD4⁺ T cell proliferation than CD1c⁺CD14⁺ DCs (FIG. 5A), all three subsets of human vaginal DCs were able to induce similar levels of naïve CD8⁺ T cell proliferation. After 7 days of co-culture of T cells and APC subsets, CD8⁺ T cells were stimulated with PMA and ionomycin in the presence of BFA. CD8⁺ T cells were then stained for intracellular IFNγ, TNFα, and IL-5 (FIG. 6B). Both LCs and CD1c⁺CD14⁻ DCs resulted in similar percentages of IFNγ⁺ and TNFα⁺ CD8⁺ T cells. CD1c⁻CD14⁺ macrophages and CD1c⁺CD14⁺ DCs resulted in lower percentages of IFNγ⁺ and TNFα⁺ CD8⁺ T cells than the other two vaginal DC subsets did (FIG. 6B). The data presented herein also shows that LCs and CD1c⁺CD14⁻ DCs were also efficient at inducing IL-5⁺CD8⁺ T cells. Data from six (IFNγ and IL-5) and 3 (TNFα) independent studies are summarized in FIG. 6E.

The patterns of intracellular cytokines expressed in CD8⁺ T cells were further analyzed in FIGS. 6C and 6D. Majority of CD8⁺ T cells induced by LCs or CD1c⁺CD14⁻ DCs are IFNγ single-positivr (FIG. 6C). However, the majority of TNFα⁺CD8⁺ T cells induced by LCs or CD1c⁺CD14⁻ DCs also express IFNγ⁺. Approximately half of the IL-5⁺CD8⁺ T cells also express IFNγ⁺ (1.94±1% IL-5⁺IFNγ⁺ vs. 1.87±2% IL-5⁺IFNγ⁻ after co-culture with LCs) (FIG. 6D). CD8⁺ T cells expressing IL-13, IL-21, or IL-17 were not detected or were present at minimal levels (<0.1%) (data not shown). Thus, it can be concluded that LCs and submucosal DCs are more potent than CD1c⁻CD14⁺ macrophages for priming naïve CD8⁺ T cell responses. The three subsets of DCs resulted in similar levels of CD8⁺ T cell proliferation. Furthermore, LCs and CD1c⁺CD14⁻ DCs are more efficient than CD1c⁺CD14⁺ DCs and CD1c⁻CD14⁺ macrophages at inducing IL-5-producing CD8⁺ T cell responses. However, the majority of IL-5⁺CD8⁺ T cells are also able to express IFNγ.

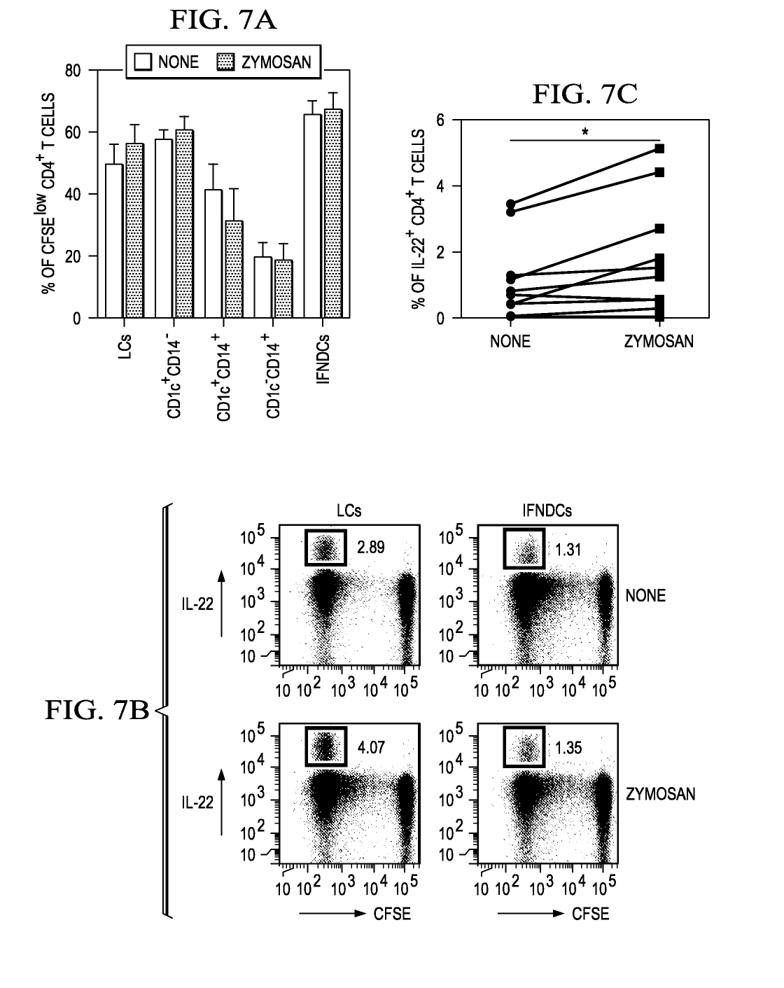

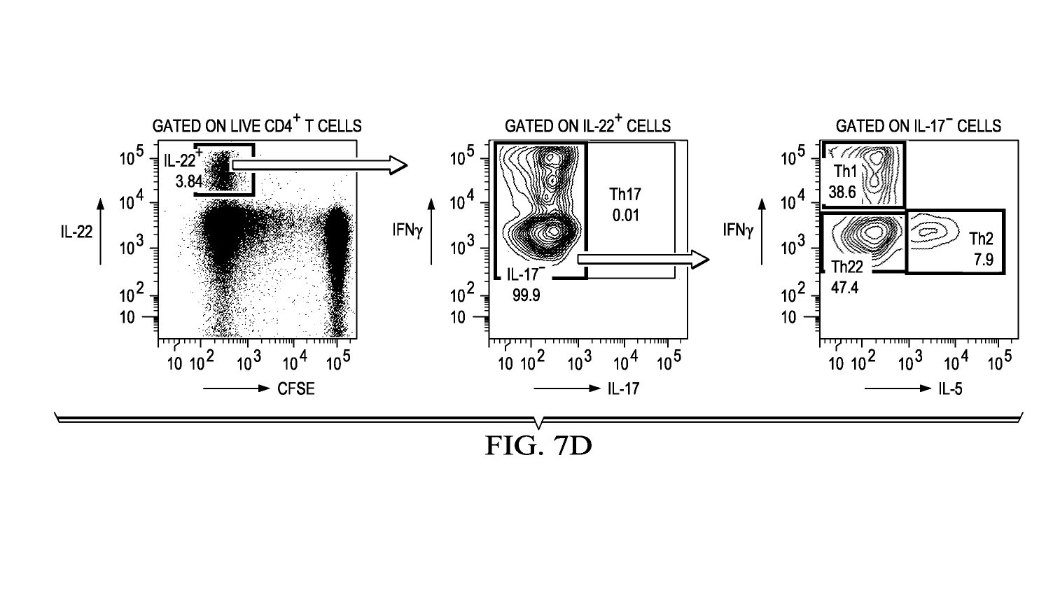

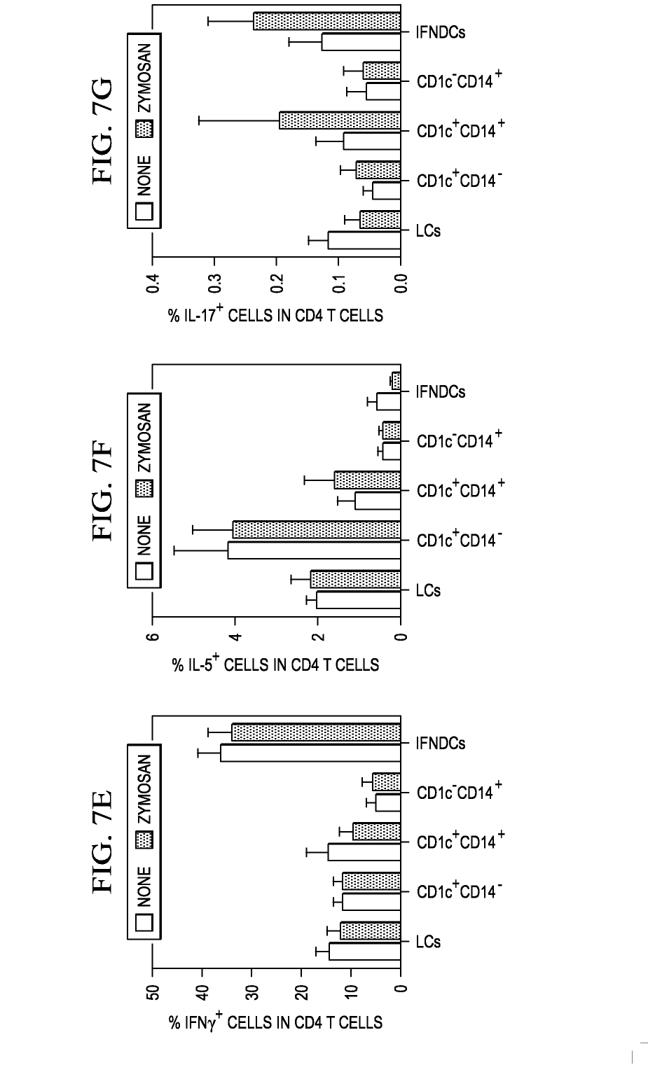

Zymosan can enhance LC-mediated IL-22-producing CD4+ T cell responses: The human vagina carries commensal microbes, including yeasts. In this study, therefore, the inventors hypothesized that yeasts could contribute to the mucosal immunity as well as the maintenance of the vaginal mucosa by acting through the APCs in the vagina. To test this, vaginal APC subsets were treated with zymosan, yeast cell wall components, and co-cultured with naïve T cells. At day 7 of the co-culture, CD4+ T cell responses induced by zymosan-activated vaginal APC subsets were assessed by measuring their proliferation and intracellular cytokine expression (FIGS. 7A-7D). None of the APC subsets treated with zymosan resulted in enhanced allogeneic naïve CD4+ T cell proliferation (FIG. 7A). In addition, zymosan did not significantly alter the numbers of IFNγ+ or IL-5+CD4+ T cells (FIGS. 7E and 7F, respectively). CD1c+CD14− and CD1c+CD14+DCs from only half of the donors tested (5 out of 10 donors) enhanced IL-17-producing CD4+ T cell responses. However, APCs from the remaining donors did not. Combined data from studies using vaginal APC subsets from 10 donors are presented in FIG. 7G. Interestingly, zymosan-treated LCs resulted in an enhanced IL-22-producing CD4+ T cell response (FIGS. 7B and 7C) which is known to contribute to mucosal immunity against infections (De Luca et al., 2010; Malmberg and Ljunggren, 2009; Vivier et al., 2009), partly by maintaining epithelial integrity and homeostasis. Minor fractions of IL-22+CD4+ T cells express IL-5 or IFNγ (right panel in FIG. 7D), indicating that they are mainly Th22 cells.

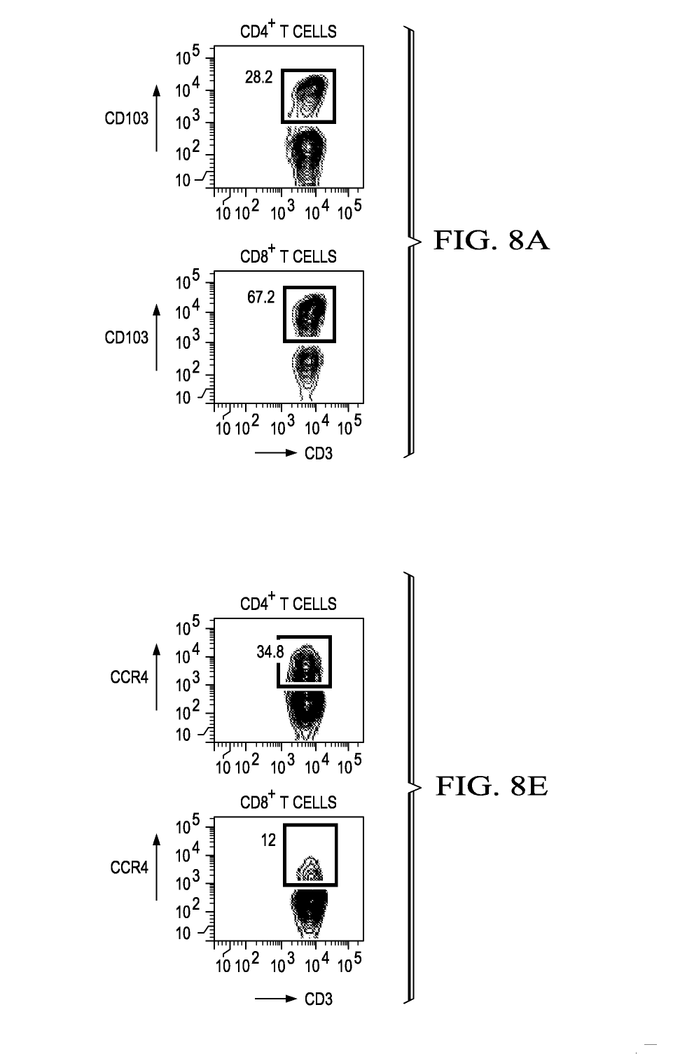

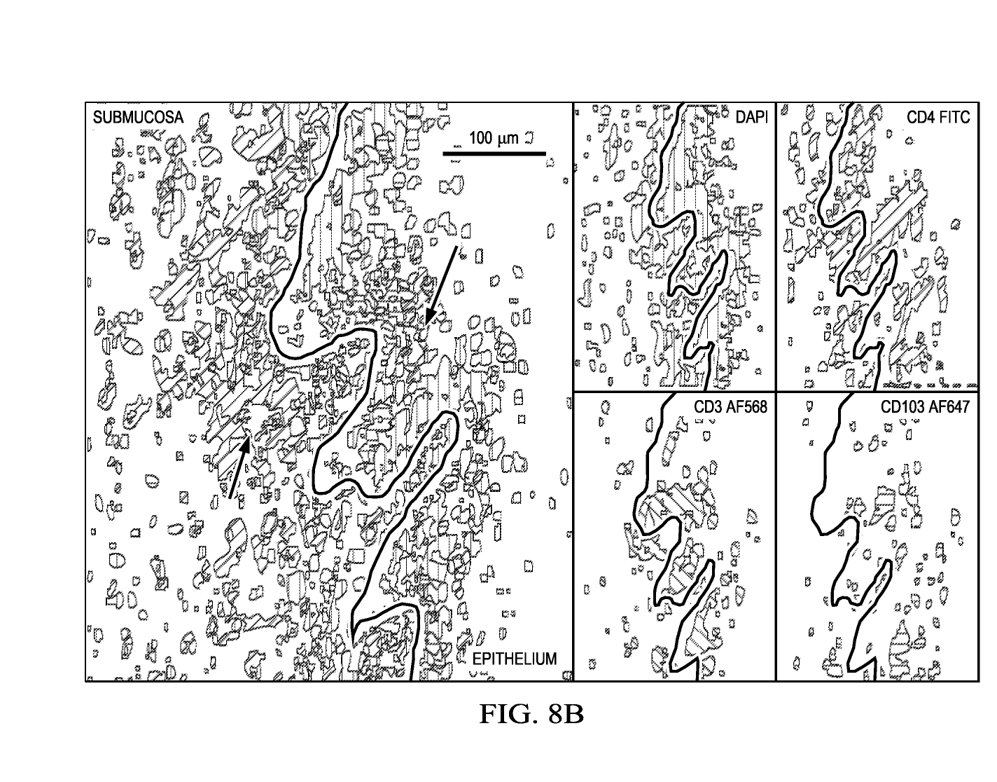

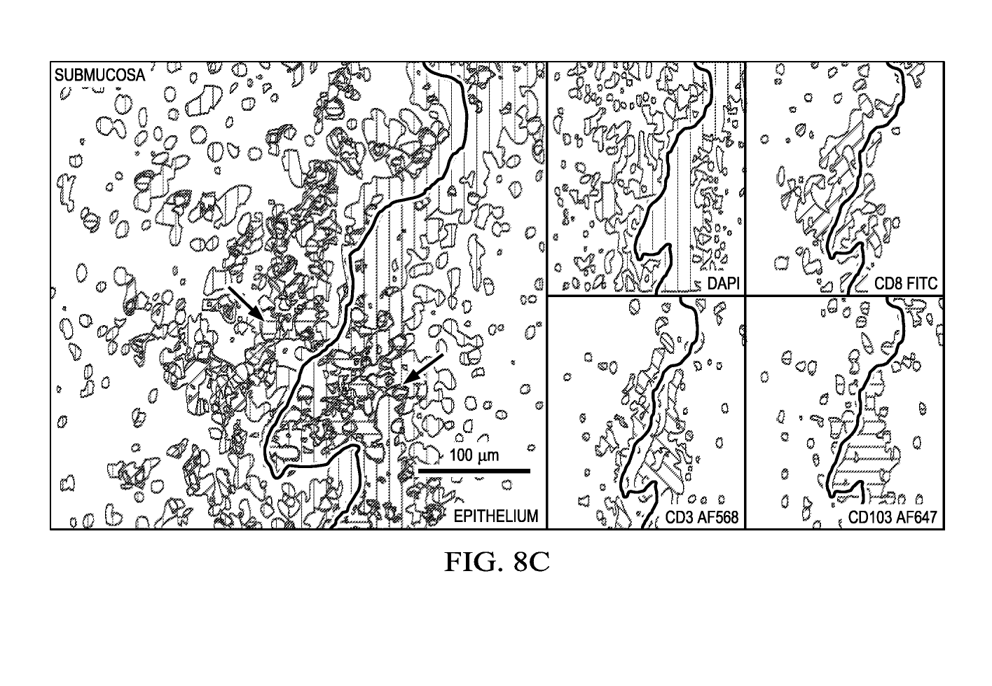

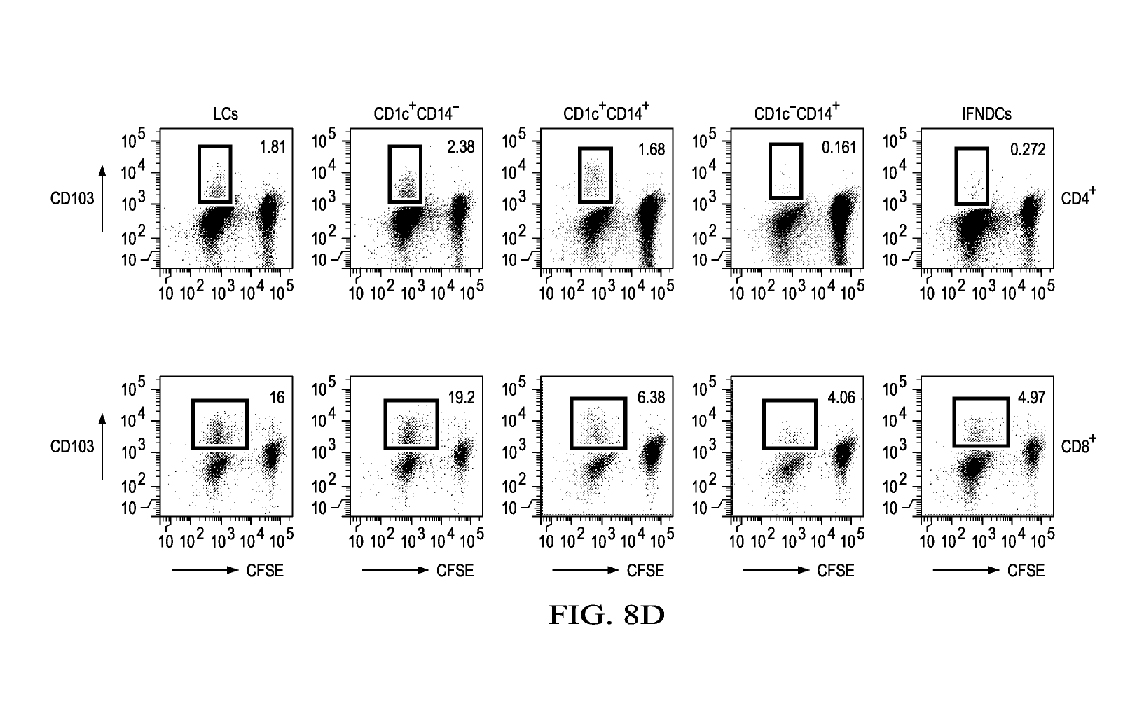

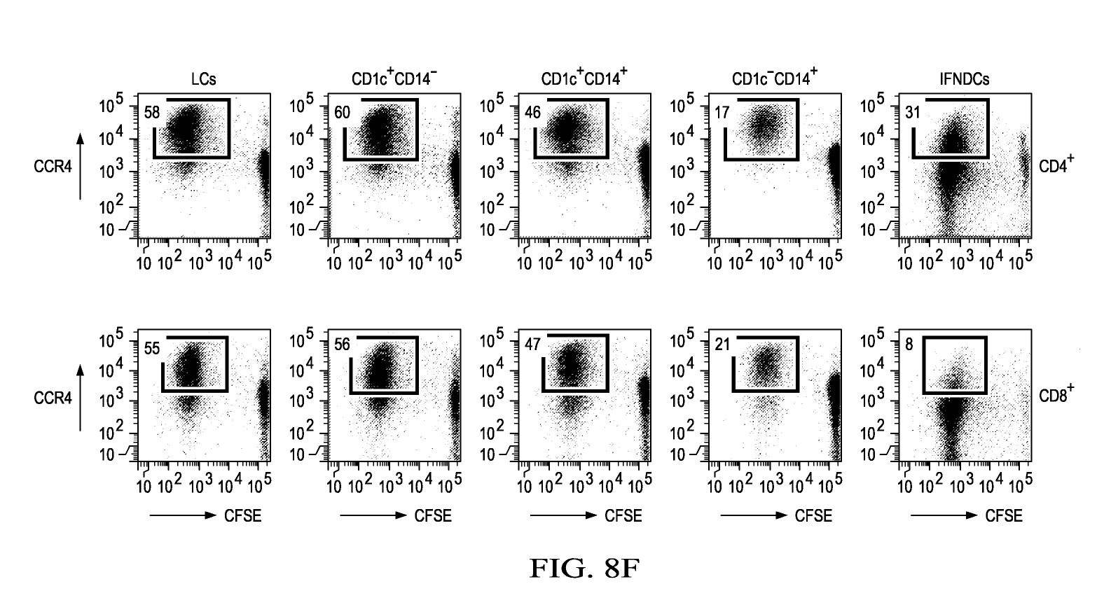

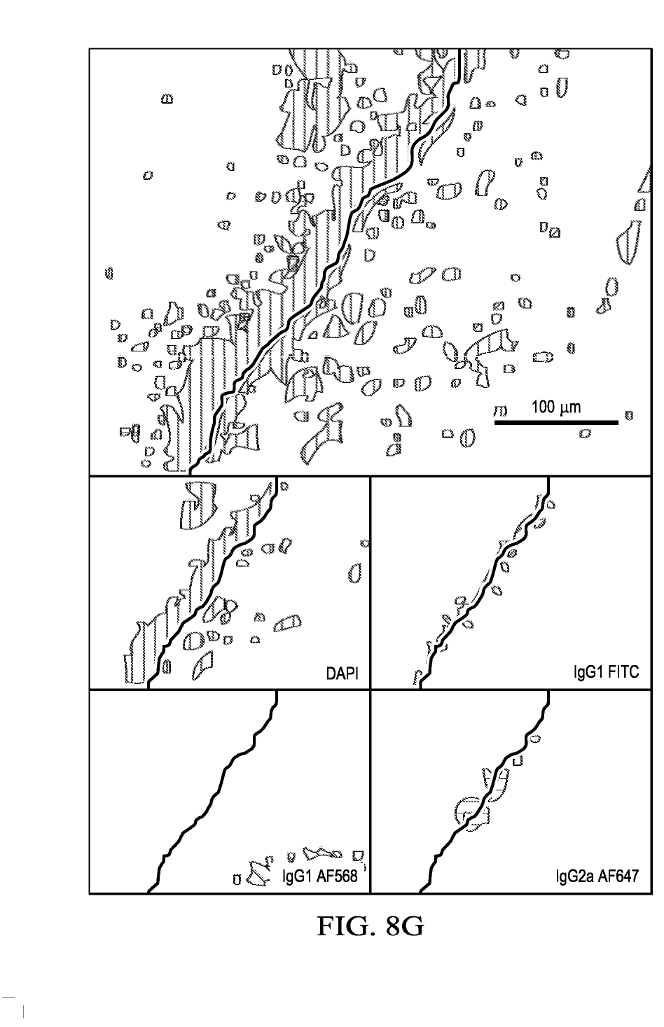

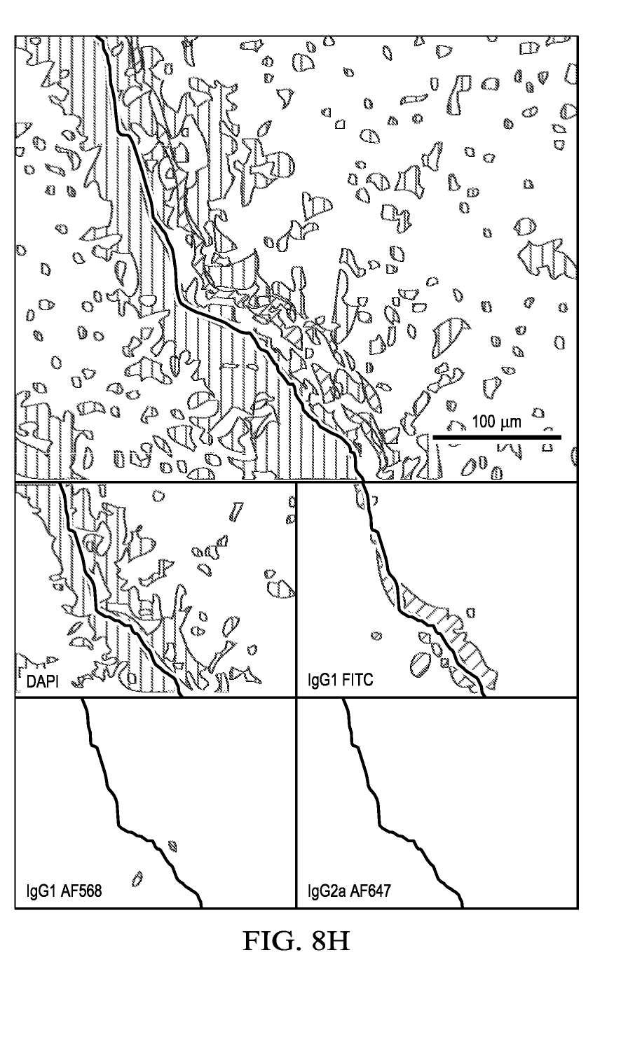

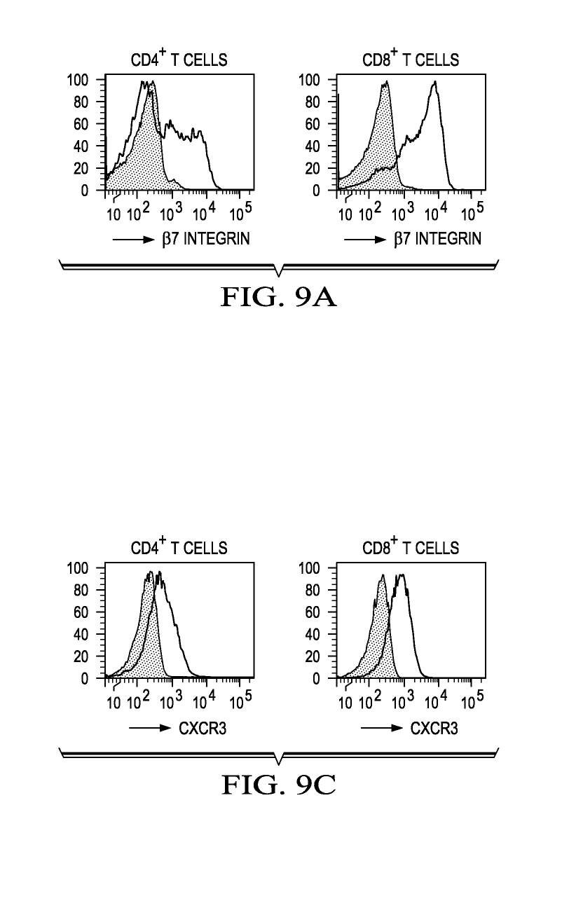

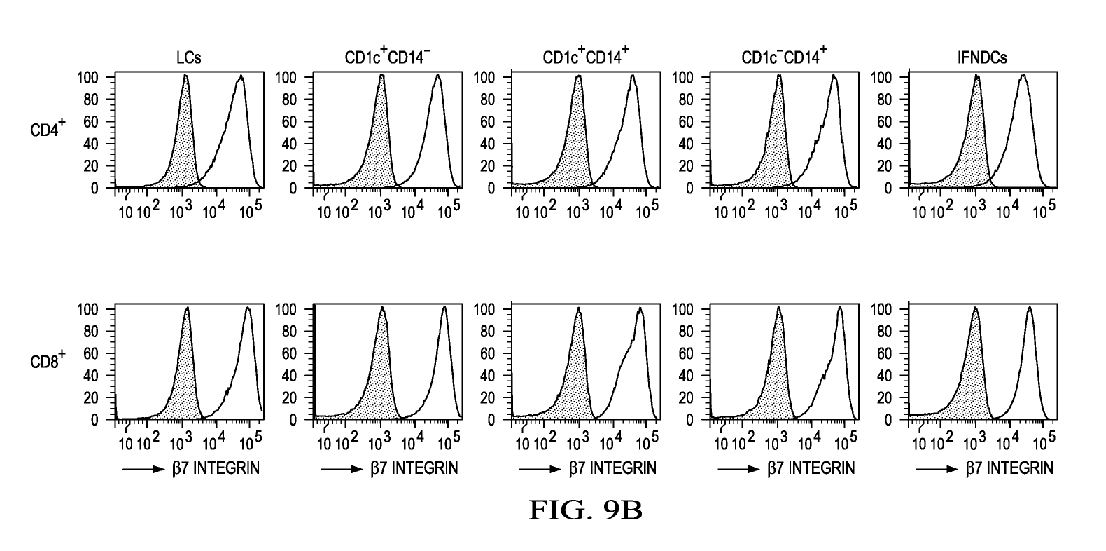

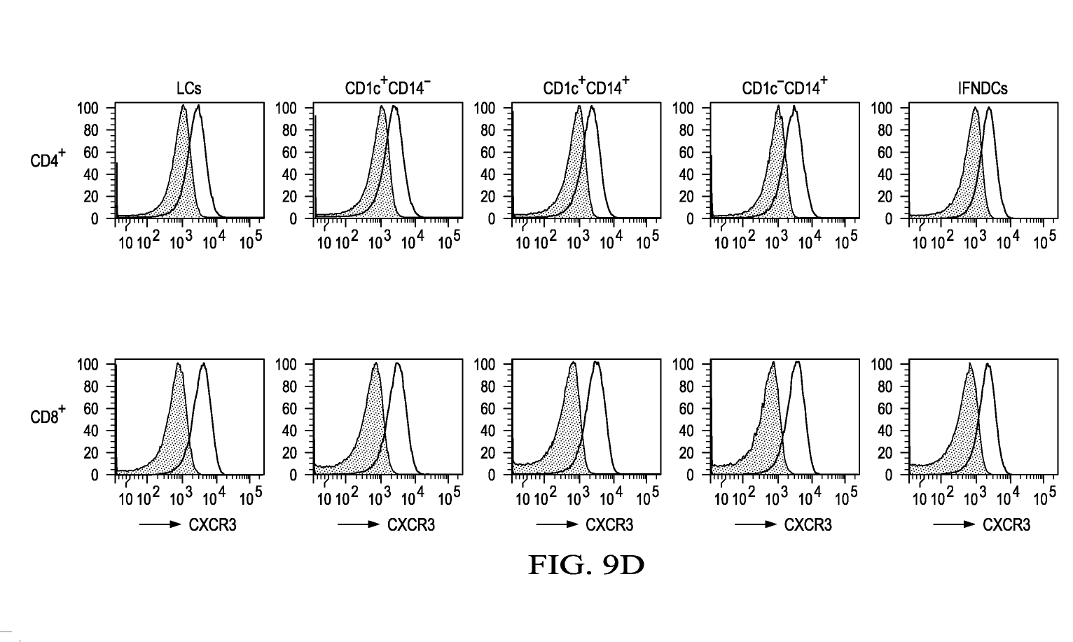

Vaginal APCs can induce CD103, β7 integrin, CCR4, and CXCR3 expression on T cells: The interaction of T cells with adhesion molecules is one of the important processes for T cell migration into local tissues. The αEβ7 (or CD103/β7) integrin allows lymphocytes to migrate into local mucosal tissues and contributes to retention within the epithelial layers of mucosa (Schon et al., 1999). FIG. 8A shows that fractions of CD4+ T cells (28.2%) and CD8+ T cells (67.2%) from human vaginal mucosa express CD103. This was further confirmed by examining CD4+ (FIG. 8B) and CD8+ T cells (FIG. 8C) localized in epithelium and submucosa of the human vagina. Tissue sections stained with isotype control antibodies for FIGS. 8B and 8C are presented in FIGS. 8G and 8H, respectively. Fractions of both CD4+ and CD8+ T cells from the vagina also expressed β7 integrin (FIG. 9A).

To determine whether vaginal APCs are able to induce CD103 and β7 integrin on the surface of T cells, CFSE-labeled naïve CD4+ and CD8+ T cells were co-cultured with APC subsets for 7 days. T cells were then stained with anti-CD103 and anti-β7 integrin antibodies. FIG. 8D shows that LCs and CD1c+CD14− DCs are able to induce CD103 on the surface of both CD4+ (upper panels) and CD8+ T cells (lower panel). CD1c+CD14+ DCs were also capable of inducing CD103, but were less potent than the other two DC subsets. Compared to the three subsets of vaginal DCs, CD1c-CD14+ Mφ and IFNDCs were less efficient for the induction of CD103 expression on both CD4+ and CD8+ T cells. All four subsets of vaginal APCs were almost equally able to induce β7 integrin on the surface of both CD4+ and CD8+ T cells (FIG. 9B).

The inventors also found that fractions of CD4+ and CD8+ T cells from vaginal mucosa express CCR4 (FIG. 8E), which had not been previously described. Interestingly, all four subsets of vaginal APCs induced high number of CCR4+CD4+ and CCR4+CD8+ T cells after 7 days of co-culture (FIG. 8F). IFNDCs also induced CCR4 expression on both CD4+ and CD8+ T cells, but less efficiently than did the vaginal APC subsets. Although the percentage of CCR4+ T cells induced by CD1c-CD14+ macrophages was less than those induced by the three subsets of vaginal DCs, the majority of CFSElow T cells induced with CD1c-CD14+ macrophages expressed CCR4.

T cells from the vaginal tissues also expressed CXCR3 (FIG. 9C). In addition, both CD4+ and CD8+ T cells co-cultured with vaginal APC subsets expressed CXCR3 on their surface (FIG. 9D). Taken together, the data presented herein demonstrate that T cells from human vagina express CD103, β7 integrin, CCR4, and CXCR3 as potential receptors for either migration into vaginal mucosa or retention of T cells in the vagina. More importantly, vaginal APCs, particularly DCs, are able to induce such receptors on the surface of both CD4+ and CD8+ T cells.

Understanding the immunology of human vagina will be crucial to overcoming the major challenges remaining in the prevention or treatment of STDs. In this study we demonstrate, for the first time, that human vaginal mucosa harbors four major myeloid-originated subsets of APCs (LCs in epithelium, CD1c+CD14− DCs, CD1c+CD14+ DCs, and CD1c-CD14+ macrophages in the submucosa) that show distinct phenotypes and functions. Although pDCs and B cells could contribute to immunity in the human vagina, as shown in mice infected with HSV-2 (Iijima et al., 2008a; Lund et al., 2003; Shen and Iwasaki, 2006), the human vagina contains minimal numbers of pDCs and B cells in a steady state.

CD207+ cells, localized mainly in the epithelium of human vagina, are defined as LCs. In addition to their morphology, CD207+ cells express CD1a, CD1c, and E-cadherin, which were also observed in skin LCs (Klechevsky et al., 2008). Although LCs and submucosal DCs express similar patterns of costimulatory molecules and chemokine receptors, only LCs express high levels of CD205, like LCs in skin (Blauvelt and Katz, 1995). Vaginal LCs do not contain Birbeck granules (Iwasaki, 2007; Parr et al., 1991). Both human skin dermis (Klechevsky et al., 2008) and vaginal submucosa contain subsets of DCs that can be subdivided based upon CD14 expression. CD1c+CD14− and CD1c+CD14+ cells are defined as submucosal DCs based upon the presence of dendrites as well as CD1c, CD11c and CD83 expression. CD1c-CD14+ macrophages express CD163 and contain large vacuoles in the cytoplasm. In addition, CD1c+CD14− DCs and CD1c+CD14+ DCs are mainly localized in the proximal site of epithelium, whereas CD1c-CD14+ macrophages are found throughout the submucosa. CCR2 expression on CD1c-CD14+ cells further support their classification as Mφ. Submucosal DCs and macrophages are also distinguished by LLRs and CD1d expression. Both CD209 and Dectin-1 are mainly expressed on the two subsets of submucosal DCs, whereas CD1d and LOX-1 were mainly expressed on CD1c-CD14+CD163+ macrophages. None of the vaginal APC subsets express CCR7, suggesting these cells would need further activation for their homing to the draining lymph nodes. However, DCs purified after in vitro migration expressed CCR7 (data not shown).

Although female hormones could influence the biology in the vagina (Kaushic et al., 2000; Kozlowski et al., 2002; Prieto and Rosenstein, 2006; Wira et al., 2002), the frequency of submucosal APCs were relatively stable compared to LCs. Epithelial layers in tissues from certain donors (less than 15%) were thinner and contain fewer numbers of LCs than in the tissues from other donors. This suggests female hormones might exert more influence on the epithelium and LCs than submucosal APCs (Wieser et al., 2001). Neither LCs or submucosal DCs from the donors tested in this study expressed estrogen or progesterone receptor (data not shown). However, a large fractions of vimentin+stromal cells express female hormone receptors, suggesting that female hormone could exert their influence on the vaginal DCs indirectly through non-APC cell types. However, the effect of female hormones in the frequency and biological function of APCs in the human vagina needs to be studied.

Some functional specialties of other tissue-resident and DCs in lymphoid organs have been previously described (Allan et al., 2006; den Haan et al., 2000; Villadangos and Schnorrer, 2007) (Allan et al., 2006; den Haan et al., 2000; Dudziak et al., 2007; Itano et al., 2003; Jaensson et al., 2008; Klechevsky et al., 2008; Maldonado-Lopez et al., 1999; Shortman and Liu, 2002; Soares et al., 2007; Villadangos and Schnorrer, 2007). In this study, the inventors demonstrate that DC subsets in the human vagina have common as well as distinct functions in directing mucosal T cell responses. Vaginal LCs and submucosal CD1c+CD14− DCs polarize naïve T cells into Th2-type, whereas CD1c+CD14+ DCs and CD1c-CD14+ macrophages polarize them toward Th1-type. These characteristics of APC subsets in the vagina are partially comparable with those of human skin DCs, where LCs and CD1a+CD14− dermal DCs can efficiently elicit Th2-type CD4+ T cell responses, while CD14+ dermal DCs elicit T follicular helper (Tfh) CD4+ T cell responses (Klechevsky et al., 2008). However, vaginal APC subsets did not induce significant levels of Tfh responses. Vaginal LCs and CD1c+CD14− DCs were able to induce naïve CD8+ T cells to express IL-5. The physiologic function of IL-5+CD8+ T cells is largely unknown. Epithelial/endothelial cells expressing MHC II can also activate CD4+ T cells responses (Hershberg et al., 1997; Taflin et al., 2011), and thus might also contribute to the immune responses in the vagina and female genital tract.

IL-22, an IL-10 family cytokine, plays an important role in antimicrobial immunity, inflammation, and tissue repair (Aujla and Kolls, 2009; Zelante et al., 2011) (De Luca et al., 2010; Zelante et al., 2011). IL-22 is produced by multiple cell types including NK cells, CD8+ T cells, Th1, Th17 and the specialized Th22 cells (De Luca et al., 2010; Malmberg and Ljunggren, 2009; Vivier et al., 2009; Zelante et al., 2011). In this study, it was found that vaginal zymosan-activated LCs, enhance IL-22-producing CD4+ T cells, which is in accordance with previous work demonstrating skin LCs can induce Th22 cells (Fujita et al., 2009). IL-22-producing CD4+ T cells induced by LCs do not express IFNγ or IL-17, suggesting they are Th22 cells. Vaginal LCs also induced CD8+ T cells to express IL-22, but the percentages of IL-22+CD8+ T cells were minimal (<1%) (data not shown). The findings of the present invention on zymosan-activated LC-mediated Th22 cell induction is particularly relevant to the protective role of vaginal LCs in enhancing innate immunity against infections. In consideration of yeasts as commensal microbes in the vagina, LCs also play an important role in the maintenance and recovery of epithelial barriers in the vagina where multiple physical or biological factors, (e.g., sexual intercourses and microbial infections followed by inflammation), could result in tissue damage.

The present inventors further demonstrate that fractions of T cells in the human vagina express, CD103, CCR4, CXCR3, and β7 integrin. CD103 is expressed on human and simian vaginal T cells (Hladik et al., 1999; Stevceva et al., 2002) and αEβ7 (CD103/(37) contribute to the recruitment of lymphocytes into local mucosa (Schon et al., 1999), especially in the vagina (Csencsits et al., 2001; Stevceva et al., 2002). More importantly, the inventors demonstrate that vaginal DCs, specifically LCs and CD1c+CD14− DCs, can efficiently induce CD103 expression on the surface of CD4+ and CD8+ T cells. All subsets of vaginal APCs are able to induce similar levels of β7 integrin and CXCR3 (Nakanishi et al., 2009) on T cells. It is further demonstrated that fractions of CD4+ and CD8+ T cells from the vagina express CCR4, a chemokine receptor expressed on Th2-type CD4+ T cells. APCs from the vagina could induce CCR4 on both CD4+ and CD8+ T cells. The numbers of CCR4+CD4+ T cells induced by LCs or CD1c+CD14−DCs were higher than those induced by CD1c+CD14+ DCs or CD1c-CD14+ macrophages. The inventors also observed that vaginal tissues secrete a significant amount of the CCR4 ligand CCL20 (data not shown). Thus, CCR4 might be another important T cell homing receptor to the vagina. CCR4 may also contribute to the retention or recruitment of APCs in the vagina, as all four subsets of vaginal APCs express high levels of CCR4. Vaginal epithelial cells can also express CCL22 (Cremel et al., 2005; Williams, 2004), a ligand for CCR6 that is expressed on the four vaginal APC subsets. Taken together, the data obtained in the present invention demonstrates that vaginal APC subsets have the unique capacity for mounting immunity in the vagina by inducing receptors that may allow effector cells to migrate into the vagina. Although vaginal epithelial cells express CCL25, CCL27, and CCL28 in a steady state (Iwasaki, 2007), T cells in the vagina from the majority of tissue donors did not express CCR9 or CCR10. However, CCR10+B cells were detected in the vagina tissues from certain donors (data not shown).

The invention described hereinabove demonstrates that human vaginal mucosa contains at least four major subsets of myeloid-derived APCs in a steady state. Each of the subsets displays common as well as unique phenotypes and functions that contribute to the immune responses in the vagina. Studies conducted herein provide foundational knowledge for understanding the immunology of human vagina and enable the design of advanced immunotherapeutics and vaccines against STDs.

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

European Patent Application No. EP0779817: Enteral Vaccines for Vaginal Infections.
Allan, R. S., J. Waithman, S. Bedoui, C. M. Jones, J. A. Villadangos, Y. Zhan, A. M. Lew, K. Shortman, W. R. Heath, and F. R. Carbone. 2006. Migratory dendritic cells transfer antigen to a lymph node-resident dendritic cell population for efficient CTL priming. Immunity 25:153-162.
Aujla, S. J., and J. K. Kolls. 2009. IL-22: a critical mediator in mucosal host defense. J Mol Med 87:451-454.
Blauvelt, A., and S. I. Katz. 1995. The skin as target, vector, and effector organ in human immunodeficiency virus disease. J Invest Dermatol 105:1225-1265.
Brown, G. D. 2006. Dectin-1: a signalling non-TLR pattern-recognition receptor. Nat Rev Immunol 6:33-43.
Cremel, M., W. Berlier, H. Hamzeh, F. Cognasse, P. Lawrence, C. Genin, J. C. Bernengo, C. Lambert, M. C. Dieu-Nosjean, and O. Delezay. 2005. Characterization of CCL20 secretion by human epithelial vaginal cells: involvement in Langerhans cell precursor attraction. J Leukoc Biol 78:158-166.
Csencsits, K. L., N. Walters, and D. W. Pascual. 2001. Cutting edge: dichotomy of homing receptor dependence by mucosal effector B cells: alpha(E) versus L-selectin. J Immunol 167:2441-2445.
De Luca, A., T. Zelante, C. D'Angelo, S. Zagarella, F. Fallarino, A. Spreca, R. G. Iannitti, P. Bonifazi, J. C. Renauld, F. Bistoni, P. Puccetti, and L. Romani. 2010. IL-22 defines a novel immune pathway of antifungal resistance. Mucosal Immuno13:361-373.
Delneste, Y., G. Magistrelli, J. Gauchat, J. Haeuw, J. Aubry, K. Nakamura, N. Kawakami-Honda, L. Goetsch, T. Sawamura, J. Bonnefoy, and P. Jeannin. 2002. Involvement of LOX-1 in dendritic cell-mediated antigen cross-presentation. Immunity 17:353-362.
den Haan, J. M., S. M. Lehar, and M. J. Bevan. 2000. CD8(⁺) but not CD8(−) dendritic cells cross-prime cytotoxic T cells in vivo. J Exp Med 192:1685-1696.
Dudziak, D., A. O. Kamphorst, G. F. Heidkamp, V. R. Buchholz, C. Trumpfheller, S. Yamazaki, C. Cheong, K. Liu, H. W. Lee, C. G. Park, R. M. Steinman, and M. C. Nussenzweig. 2007. Differential antigen processing by dendritic cell subsets in vivo. Science 315:107-111.
Echchannaoui, H., M. Bianchi, D. Baud, M. Bobst, J. C. Stehle, and D. Nardelli-Haefliger. 2008. Intravaginal immunization of mice with recombinant Salmonella enterica serovar Typhimurium expressing human papillomavirus type 16 antigens as a potential route of vaccination against cervical cancer. Infect Immun 76:1940-1951.
Figdor, C. G., Y. van Kooyk, and G. J. Adema. 2002. C-type lectin receptors on dendritic cells and Langerhans cells. Nat Rev Immunol 2:77-84.
Fujita, H., K. E. Nograles, T. Kikuchi, J. Gonzalez, J. A. Carucci, and J. G. Krueger. 2009. Human Langerhans cells induce distinct IL-22-producing CD4⁺ T cells lacking IL-17 production. Proc Natl Acad Sci USA 106:21795-21800.
Geijtenbeek, T. B., and S. I. Gringhuis. 2009. Signalling through C-type lectin receptors: shaping immune responses. Nat Rev Immunol 9:465-479.
Geijtenbeek, T. B., R. Torensma, S. J. van Vliet, G. C. van Duijnhoven, G. J. Adema, Y. van Kooyk, and C. G. Figdor. 2000. Identification of DC-SIGN, a novel dendritic cell-specific ICAM-3 receptor that supports primary immune responses. Cell 100:575-585.
Geijtenbeek, T. B., S. J. van Vliet, A. Engering, B. A. t Hart, and Y. van Kooyk. 2004. Self- and nonself-recognition by C-type lectins on dendritic cells. Annu Rev Immunol 22:33-54.
Gupta, S., R. Janani, Q. Bin, P. Luciw, C. Greer, S. Perri, H. Legg, J. Donnelly, S. Barnett, D. O'Hagan, J. M. Polo, and M. Vajdy. 2005. Characterization of human immunodeficiency virus Gag-specific gamma interferon-expressing cells following protective mucosal immunization with alphavirus replicon particles. J Virol 79:7135-7145.
Haneberg, B., D. Kendall, H. M. Amerongen, F. M. Apter, J. P. Kraehenbuhl, and M. R. Neutra. 1994. Induction of specific immunoglobulin A in the small intestine, colon-rectum, and vagina measured by a new method for collection of secretions from local mucosal surfaces. Infect Immun 62:15-23.
Hedges, S. R., D. A. Sibley, M. S. Mayo, E. W. Hook, 3rd, and M. W. Russell. 1998. Cytokine and antibody responses in women infected with Neisseria gonorrhoeae: effects of concomitant infections. J Infect Dis 178:742-751.
Hershberg, R. M., P. E. Framson, D. H. Cho, L. Y. Lee, S. Kovats, J. Beitz, J. S. Blum, and G. T. Nepom. 1997. Intestinal epithelial cells use two distinct pathways for HLA class II antigen processing. Clin Invest 100:204-215.
Hladik, F., G. Lentz, E. Delpit, A. McElroy, and M. J. McElrath. 1999. Coexpression of CCR5 and IL-2 in human genital but not blood T cells: implications for the ontogeny of the CCR5⁺ Th1 phenotype. J Immunol 163:2306-2313.
Hladik, F., P. Sakchalathorn, L. Ballweber, G. Lentz, M. Fialkow, D. Eschenbach, and M. J. McElrath. 2007. Initial events in establishing vaginal entry and infection by human immunodeficiency virus type-1. Immunity 26:257-270.
Hu, J., M. Pope, C. Brown, U. O'Doherty, and C. J. Miller. 1998. Immunophenotypic characterization of simian immunodeficiency virus-infected dendritic cells in cervix, vagina, and draining lymph nodes of rhesus monkeys. Lab Invest 78:435-451.

Huang, F. P., N. Platt, M. Wykes, J. R. Major, T. J. Powell, C. D. Jenkins, and G. G. MacPherson. 2000. A discrete subpopulation of dendritic cells transports apoptotic intestinal epithelial cells to T cell areas of mesenteric lymph nodes. J Exp Med 191:435-444.

Iijima, N., M. M. Linehan, M. Zamora, D. Butkus, R. Dunn, M. R. Kehry, T. M. Laufer, and A. Iwasaki. 2008a. Dendritic cells and B cells maximize mucosal Th1 memory response to herpes simplex virus. J Exp Med 205:3041-3052.

Iijima, N., J. M. Thompson, and A. Iwasaki. 2008b. Dendritic cells and macrophages in the genitourinary tract. Mucosal Immunol 1:451-459.
Itano, A. A., S. J. McSorley, R. L. Reinhardt, B. D. Ehst, E. Ingulli, A. Y. Rudensky, and M. K. Jenkins 2003. Distinct dendritic cell populations sequentially present antigen to CD4 T cells and stimulate different aspects of cell-mediated immunity. Immunity 19:47-57.
Iwasaki, A. 2007. Mucosal dendritic cells. Annu Rev Immunol 25:381-418.
Iwasaki, A. 2010. Antiviral immune responses in the genital tract: clues for vaccines. Nat Rev Immunol 10:699-711.
Jaensson, E., H. Uronen-Hansson, O. Pabst, B. Eksteen, J. Tian, J. L. Coombes, P. L. Berg, T. Davidsson, F. Powrie, B. Johansson-Lindbom, and W. W. Agace. 2008. Small intestinal CD103+ dendritic cells display unique functional properties that are conserved between mice and humans. J Exp Med 205:2139-2149.
Jameson, B., F. Baribaud, S. Pohlmann, D. Ghavimi, F. Mortari, R. W. Doms, and A. Iwasaki. 2002. Expression of DC-SIGN by dendritic cells of intestinal and genital mucosae in humans and rhesus macaques. J Virol 76:1866-1875.
Johansson, C., and B. L. Kelsall. 2005. Phenotype and function of intestinal dendritic cells. Semin Immunol 17:284-294.
Jongbloed, S. L., A. J. Kassianos, K. J. McDonald, G. J. Clark, X. Ju, C. E. Angel, C. J. Chen, P. R. Dunbar, R. B. Wadley, V. Jeet, A. J. Vulink, D. N. Hart, and K. J. Radford. 2010. Human CD141+ (BDCA-3)+dendritic cells (DCs) represent a unique myeloid DC subset that cross-presents necrotic cell antigens. J Exp Med 207:1247-1260.
Kaushic, C., F. Zhou, A. D. Murdin, and C. R. Wira. 2000. Effects of estradiol and progesterone on susceptibility and early immune responses to Chlamydia trachomatis infection in the female reproductive tract. Infect Immun 68:4207-4216.
Klechevsky, E., R. Morita, M. Liu, Y. Cao, S. Coquery, L. Thompson-Snipes, F. Briere, D. Chaussabel, G. Zurawski, A. K. Palucka, Y. Reiter, J. Banchereau, and H. Ueno. 2008. Functional Specializations of Human Epidermal Langerhans Cells and CD14(+) Dermal Dendritic Cells. Immunity.
Kozlowski, P. A., S. Cu-Uvin, M. R. Neutra, and T. P. Flanigan. 1997. Comparison of the oral, rectal, and vaginal immunization routes for induction of antibodies in rectal and genital tract secretions of women. Infect Immun 65:1387-1394.
Kozlowski, P. A., S. B. Williams, R. M. Lynch, T. P. Flanigan, R. R. Patterson, S. Cu-Uvin, and M. R. Neutra. 2002. Differential induction of mucosal and systemic antibody responses in women after nasal, rectal, or vaginal immunization: influence of the menstrual cycle. J Immunol 169:566-574.
Kwant, A., and K. L. Rosenthal. 2004. Intravaginal immunization with viral subunit protein plus CpG oligodeoxynucleotides induces protective immunity against HSV-2. Vaccine 22:3098-3104.
Lee, H. K., M. Zamora, M. M. Linehan, N. Iijima, D. Gonzalez, A. Haberman, and A. Iwasaki. 2009. Differential roles of migratory and resident DCs in T cell priming after mucosal or skin HSV-1 infection. J Exp Med 206:359-370.
Lindqvist, M., J. Persson, K. Thorn, and A. M. Harandi. 2009. The mucosal adjuvant effect of alpha-galactosylceramide for induction of protective immunity to sexually transmitted viral infection. J Immunol 182:6435-6443.
Lund, J., A. Sato, S. Akira, R. Medzhitov, and A. Iwasaki. 2003. Toll-like receptor 9-mediated recognition of Herpes simplex virus-2 by plasmacytoid dendritic cells. J Exp Med 198:513-520.
Maldonado-Lopez, R., T. De Smedt, P. Michel, J. Godfroid, B. Pajak, C. Heirman, K. Thielemans, O. Leo, J. Urbain, and M. Moser. 1999. CD8alpha+ and CD8alpha− subclasses of dendritic cells direct the development of distinct T helper cells in vivo. J Exp Med 189:587-592.
Malmberg, K. J., and H. G. Ljunggren. 2009. Spotlight on IL-22-producing NK cell receptor-expressing mucosal lymphocytes. Nat Immunol 10:11-12.
Mestecky, J., Z. Moldoveanu, and M. W. Russell. 2005. Immunologic uniqueness of the genital tract: challenge for vaccine development. Am J Reprod Immunol 53:208-214.
Mestecky, J., Z. Moldoveanu, P. D. Smith, Z. Hel, and R. C. Alexander. 2009. Mucosal immunology of the genital and gastrointestinal tracts and HIV-1 infection. J Reprod Immunol 83:196-200.
Miller, C. J., McChesney. M., and Moore, P. F. 1992. Langerhans cells, macrophages and lymphocyte subsets in the cervix and vagina of rhesus macaques. Lab Invest 67(5):628-634.
Nakanishi, Y., B. Lu, C. Gerard, and A. Iwasaki. 2009. CD8(+) T lymphocyte mobilization to virus-infected tissue requires CD4(+) T-cell help. Nature 462:510-513.
Ohara-Inagaki, K., Daikoku, T., Goshima, F., and Nishiyama, Y. 2000. Impaired induction of protective immunity by highly virulent herpes simplex virus type 2 in a murine model of genital herpes. Archives of Virology 145(10): 1989-2002.
Parr, M. B., L. Kepple, and E. L. Parr. 1991. Langerhans cells phagocytose vaginal epithelial cells undergoing apoptosis during the murine estrous cycle. Biol Reprod 45:252-260.
Parr, M. B., and E. L. Parr. 1991. Langerhans cells and T lymphocyte subsets in the murine vagina and cervix. Biol Reprod 44:491-498.
Poulin, L. F., M. Salio, E. Griessinger, F. Anjos-Afonso, L. Craciun, J. L. Chen, A. M. Keller, O. Joffre, S. Zelenay, E. Nye, A. Le Moine, F. Faure, V. Donckier, D. Sancho, V. Cerundolo, D. Bonnet, and C. Reis e Sousa. 2010. Characterization of human DNGR-1+BDCA3+ leukocytes as putative equivalents of mouse CD8alpha+dendritic cells. J Exp Med 207:1261-1271.
Prieto, G. A., and Y. Rosenstein. 2006. Oestradiol potentiates the suppressive function of human CD4 CD25 regulatory T cells by promoting their proliferation. Immunology 118:58-65.
Rosenthal, K. L., and W. S. Gallichan. 1997. Challenges for vaccination against sexually-transmitted diseases: induction and long-term maintenance of mucosal immune responses in the female genital tract. Semin. Immunol. 9:303-314.
Schon, M. P., A. Arya, E. A. Murphy, C. M. Adams, U. G. Strauch, W. W. Agace, J. Marsal, J. P. Donohue, H. Her, D. R. Beier, S. Olson, L. Lefrancois, M. B. Brenner, M. J. Grusby, and C. M. Parker. 1999. Mucosal T lymphocyte numbers are selectively reduced in integrin alpha E (CD103)-deficient mice. J Immunol 162:6641-6649.
Seavey, M. M., and Mosmann, T. R. 2009. Estradiol-induced vaginal mucus inhibits antigen penetration and CD8+ T cell priming in response to intravaginal immunization. Vaccine 27(17):2342-2349.
Serbina, N. V., T. Jia, T. M. Hohl, and E. G. Pamer. 2008. Monocyte-mediated defense against microbial pathogens. Annu Rev Immunol 26:421-452.
Shen, H., and A. Iwasaki. 2006. A crucial role for plasmacytoid dendritic cells in antiviral protection by CpG ODN-based vaginal microbicide. J Clin Invest 116:2237-2243.
Shortman, K., and Y. J. Liu. 2002. Mouse and human dendritic cell subtypes. Nat Rev Immunol 2:151-161.
Soares, H., H. Waechter, N. Glaichenhaus, E. Mougneau, H. Yagita, O. Mizenina, D. Dudziak, M. C. Nussenzweig, and R. M. Steinman. 2007. A subset of dendritic cells induces CD4+ T cells to produce IFN-gamma by an IL-12-independent but CD70-dependent mechanism in vivo. J Exp Med 204:1095-1106.
Stevceva, L., B. Kelsall, J. Nacsa, M. Moniuszko, Z. Hel, E. Tryniszewska, and G. Franchini 2002. Cervicovaginal lamina propria lymphocytes: phenotypic characterization and their importance in cytotoxic T-lymphocyte responses to simian immunodeficiency virus SIVmac251. J Virol 76:9-18.
Taflin, C., B. Favier, J. Baudhuin, A. Savenay, P. Hemon, A. Bensussan, D. Charron, D. Glotz, and N. Mooney. 2011. Human endothelial cells generate Th17 and regulatory T cells under inflammatory conditions. Proc Natl Acad Sci USA 108:2891-2896.
Valladeau, J., V. Duvert-Frances, J. J. Pin, M. J. Kleijmeer, S. Ait-Yahia, O. Ravel, C. Vincent, F. Vega, Jr., A. Helms, D. Gorman, S. M. Zurawski, G. Zurawski, J. Ford, and S. Saeland. 2001. Immature human dendritic cells express asialoglycoprotein receptor isoforms for efficient receptor-mediated endocytosis. J Immunol 167:5767-5774.
Villadangos, J. A., and P. Schnorrer. 2007. Intrinsic and cooperative antigen-presenting functions of dendritic-cell subsets in vivo. Nat Rev Immunol 7:543-555.
Vivier, E., H. Spits, and T. Cupedo. 2009. Interleukin-22-producing innate immune cells: new players in mucosal immunity and tissue repair? Nat Rev Immunol 9:229-234.
Wassen, L., K. Schon, J. Holmgren, M. Jertborn, and N. Lycke. 1996. Local intravaginal vaccination of the female genital tract. Scand J Immuno144:408-414.
Wieser, F., J. Hosmann, W. Tschugguel, K. Czerwenka, R. Sedivy, and J. C. Huber. 2001. Progesterone increases the number of Langerhans cells in human vaginal epithelium. Fertil Steril 75:1234-1235.
Williams, I. R. 2004. Chemokine receptors and leukocyte trafficking in the mucosal immune system. Immunol Res 29:283-292.
Wira, C. R., M. A. Roche, and R. M. Rossoll. 2002. Antigen presentation by vaginal cells: role of TGFbeta as a mediator of estradiol inhibition of antigen presentation. Endocrinology 143:2872-2879.
Yuan, W., A. Dasgupta, and P. Cresswell. 2006. Herpes simplex virus evades natural killer T cell recognition by suppressing CD1d recycling. Nat Immunol 7:835-842.
Zaba, L. C., J. Fuentes-Duculan, R. M. Steinman, J. G. Krueger, and M. A. Lowes. 2007. Normal human dermis contains distinct populations of CD11c+BDCA-1+ dendritic cells and CD163+FXIIIA+ macrophages. J Clin Invest 117:2517-2525.
Zelante, T., R. Iannitti, A. De Luca, and L. Romani. 2011. IL-22 in antifungal immunity. Eur J Immunol 41:270-275.
Zhao, X., E. Deak, K. Soderberg, M. Linehan, D. Spezzano, J. Zhu, D. M. Knipe, and A. Iwasaki. 2003. Vaginal submucosal dendritic cells, but not Langerhans cells, induce protective Th1 responses to herpes simplex virus-2. J Exp Med 197:153-162.

What is claimed is:

1. An isolated immunogenic composition comprising at least one subset of antigen presenting cells (APCs), wherein the APCs possess a distinct phenotype and the subset is selected from at least one of Langerhans cells (LCs) E-cadherin⁺CD207⁺CD205⁺, vaginal CD1c⁺CD14⁻DC− ASGPR⁺CD209^{+/-}Dectin-1^{+/-} dendritic cells (DCs), vaginal CD1c⁺CD14⁺CD209^{+/-}DC-ASGPR^{+/-} DCs, CD1c⁻CD14⁺CD163⁺CD209⁺ DC-ASGPR^{+/-}Dectin-1^{+/-}LOX-1⁺CD1d⁺macrophages, or vaginal CD1c⁻CD14⁻DCs.

2. The composition of claim 1, wherein the APCs are myeloid-originated APCs.

3. The composition of claim 1, wherein the APCs are isolated from a vaginal tissue or a vaginal mucosa from a human or animal subject.

4. The composition of claim 1, wherein the composition induces proliferation of one or more T cells towards a Th-1 type, or a Th2-type, or both.

5. The composition of claim 4, wherein T cell proliferation results in an induction in expression of mucosal homing receptors, CD103, β7 integrin, CCR4, CXCR3, or any combinations thereof by the T cells.

6. A composition comprising at least one antigen presenting cell (APC) subset, wherein the composition comprises at least one of:
one or more isolated vaginal Langerhans cells (LCs) E-cadherin⁺CD207⁺CD205⁺, wherein the LCs express one or more surface molecules selected from the group consisting of CD11a, E-cadherin, or both, CD86, CD83 or any combinations thereof;
one or more isolated vaginal CD1c⁺CD14⁻ DC-ASGPR′CD209^{+/-}Dectin-1^{+/-} dendritic cells (DCs), wherein the CD1c⁺CD14⁻ DCs express CD86, CD83, or both;
one or more isolated vaginal CD1c⁺CD14⁺ CD209^{+/-}DC-ASGPR^{+/-}DCs, wherein the CD1c⁺CD14⁺ DCs express CD86, CD83, or both; and
one or more isolated vaginal CD1c⁻CD14⁺CD163⁺CD209⁺ DC-ASGPR^{+/-}Dectin-1^{+/-}LOX-1⁺CD1d⁺ macrophages, wherein the CD1c⁻CD14⁺ macrophages express CD86, CD163, or both.

7. The composition of claim 6, wherein the composition induces proliferation of one or more T cells towards Th-1 type, Th2-type, or both.

8. The composition of claim 7, wherein T cell proliferation results in an induction in expression of mucosal homing receptors, CD103, β7 integrin, CCR4, CXCR3, or any combinations thereof by the T cells.

9. An immunostimulatory composition for generating a vaginal immune response, for a prophylaxis, a therapy or any combination thereof in a human or animal subject comprising:
one or more isolated vaginal anti-dendritic cell (DC)-specific antibodies or fragments thereof directed towards one or more specific vaginal DC subsets/macrophages loaded or chemically coupled with one or more antigenic peptides, wherein the antigenic peptides are representative of one or more epitopes of the one or more antigens implicated or involved in a disease or a condition against which the immune response, the prophylaxis, the therapy, or any combination thereof is desired, wherein the antibodies or fragments are directed towards one or more antigens selected from the group consisting of E-cadherin⁺, CD207⁺, CD205⁺ CD1c⁺, CD14⁻, DC-ASGPR^{+/-}, CD209^{+/-}, Dectin-1^{+/-}, CD86, CD83, CD209^{+/-}, CD1c^{-x, CD}14⁺, CD163⁺, LOX-1, CD1d⁺, CD1c⁻, CD14⁻, CD103, β7 integrin, CCR4, CXCR3, and any combinations thereof; and
a pharmaceutically acceptable carrier, wherein the composition is effective to produce the vaginal immune response, for prophylaxis, for therapy or any combination thereof in the human or animal subject in need of vaginal immunostimulation.

10. The composition of claim 9, wherein the DC subsets/macrophages are selected from the group consisting of Langerhans cells (LCs) E-cadherin⁺CD207⁺CD205⁺, vaginal CD1c⁺CD14⁻ DC-ASGPR⁺CD209^{+/-}Dectin-1^{+/-} DCs, vaginal CD1c⁺CD41⁺ CD209^{+/-} DC-ASGPR^{+/-} DCs, CD1c⁻CD14⁺ CD163⁺CD209⁺DC-ASGPR^{+/-}Dectin-1^{+/-}LOX-1⁺CD1d⁻ macrophages, CD1c⁻CD14⁻ DCs, and any combinations thereof.

11. The composition of claim 9, wherein the antigenic peptides comprise human immunodeficiency virus (HIV) antigens and gene products selected from the group consisting of gag, pol, and env genes, the Nef protein, reverse transcriptase, string of HIV peptides (Hipo5), a HIVgag-derived p24-PLA HIV gag p24 (gag), and other HIV components, cytomegaloviral antigens, herpes simplex viral antigens, human papilloma virus (HPV) E6 and E7 antigens, antigens from bacteria and fungi selected from the group consisting of Prevotella bivia, Prevotella melaminogenica, Gardnerella vaginalis, Trichomonas vaginalis, Mycoplasma hominis, Mobiluncus species, Neisseria gonorrhoeae, Chlamydia trachomatis, Ureaplasma urealyticus, Candida species, Streptococcus species, and Enterobacteriaceae, and cancer peptides selected from tumor associated antigens comprising antigens from genitourinary tumors such cervix, uterus, ovarian cancer, vaginal cancer, head and neck cancers caused by HPV infection, or combinations and modifications thereof.

12. The composition of claim 9, wherein the anti-DC-specific antibody is humanized.

13. The composition of claim 9, wherein the composition is adapted for intravaginal administration.

14. The composition of claim 9, wherein T cell proliferation results in an induction in expression of mucosal homing receptors, CD103, β7 integrin, CCR4, CXCR3, or any combinations thereof by the T cells.

15. A method for increasing effectiveness of antigen presentation by a vaginal antigen presenting cell (APC) in vitro or in vivo comprising:
contacting one or more isolated vaginal dendritic cell (DC) subsets/macrophages with a composition in vitro or administering the composition to a human or animal subject, selected from at least one of Langerhans cells (LCs) E-cadherin⁺CD207⁺CD205⁺, vaginal CD1c⁺CD14⁻DC-ASGPR⁺CD209^{+/-}Dectin-1^{+/-} dendritic cells (DCs), vaginal CD1c⁺CD14⁺CD209^{+/-}DC-ASGPR^{+/-} DCs, CD1c⁻CD14⁺CD163⁺CD209⁺CD-ASGPR^{+/-}Dectin-1^{+/-}LOX-1⁺CD1d⁺ macrophages, or CD1c⁻CD14⁻ DCs, and wherein the composition comprises:
one or more vaginal anti-dendritic cell (DC)-specific antibodies or fragments thereof directed towards one or more specific vaginal DC subsets/macrophages, wherein the antibodies or fragments are directed towards one or more antigens selected from the group consisting of E-cadherin⁺, CD207⁺, CD205⁺ CD1c⁺, CD14⁻, DC-ASGPR^{+/-}, CD209^{+/-}, Dectin-1^{+/-}, CD86, CD83, CD209^{+/-}, CD1c⁻, CD14⁺, CD163⁺, LOX-1, CD1d⁺, CD1c⁻, CD14⁻, CD103, β7 integrin, CCR4, CXCR3, and any combinations thereof; and
one or more native or engineered antigenic peptides chemically coupled or linked to the vaginal DC-specific antibody or fragment to form an antibody-antigen conjugate;
measuring a level of one or more agents following contact with the one or more vaginal DC subsets/macrophages in vitro or in a biological sample obtained from the human or animal subject, wherein the agents are selected from the group consisting of IFN-γ, TNF-α, IL-5, IL-17, and IL-13; and
determining increased effectiveness of antigen presentation by the conjugate, wherein a change in the level of the one or more agents is indicative of the increase in the effectiveness antigen presentation by the vaginal APCs.

16. A method for increasing effectiveness of antigen presentation by one or more dendritic cells (DCs) in a human subject comprising the steps of:
isolating one or more DCs or DC subsets from the human subject, wherein the DCs or the DC subsets are isolated from a vaginal tissue or a vaginal mucosa in the human subject;
exposing the isolated vaginal DCs or DC subsets to activating amounts of an immunostimulatory composition or a vaccine comprising:
one or more vaginal anti-dendritic cell (DC)-specific antibodies or fragments thereof directed towards one or more specific vaginal DC subsets/macrophages, wherein the antibodies or fragments are directed towards one or more antigens selected from the group consisting of E-cadherin⁺, CD207⁺, CD205⁺CD1c⁺, CD14⁻, DC-ASGPR^{+/-}, CD209^{+/-}, Dectin-1^{+/-}, CD86, CD83, CD209^{+/-}, CD1c⁻, CD14⁺ CD163⁺, LOX-1, CD1d⁺, CD1c⁻, CD14⁻, CD103, β7 integrin, CCR4, CXCR3, and any combinations thereof;
one or more antigenic peptides loaded or chemically coupled with the DC-specific antibodies or fragments thereof; and
a pharmaceutically acceptable carrier to form an activated complex; and
reintroducing the activated DC complex into the human subject.

17. The method of claim 16, further comprising the optional step of measuring a level of one or more agents selected from the group consisting of IFN-γ, TNF-α, IL-5, IL-17, and IL-13, wherein a change in the level of the one or more agents is indicative of the increase in the effectiveness of the one or more DCs or DC subsets.

18. The method of claim 16, further comprising the optional steps of:
adding one or more Toll-Like Receptor (TLR) agonist which is selected from the group consisting of TLR1, TLR2, TLR3, TLR4, TLR5, TLR6, TLR7, and TLR8 agonists;
adding one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines or combinations and modifications thereof to activated complex prior to exposing the DCs or DC subsets; and
adding one or more optional anti-DC-specific antibodies or fragments thereof selected from antibodies specifically binding to MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASGPR.

19. The method of claim 16, wherein the antigenic peptides comprise antigens produced by organisms selected from the group consisting of Prevotella bivia, Prevotella melaminogenica, Gardnerella vaginalis, Trichomonas vaginalis, Mycoplasma hominis, Mobiluncus species, Neisseria gonorrhoeae, Chlamydia trachomatis, Ureaplasma urealyticus, Candida species, Treponema pallidum, Streptococcus species, and Enterobacteriaceae, tumor associated antigens comprising antigens from genitourinary tumors such cervix, uterus, ovarian cancer, vaginal cancer, or combinations and modifications thereof, and human immunodeficiency virus (HIV) antigens and gene products selected from the group consisting of gag, pol, and env genes, the Nef protein, reverse transcriptase, string of HIV peptides (Hipo5), a HIV gag-derived p24-PLA HIV gag p24 (gag), and other HIV components, cytomegaloviral antigens, herpes simplex viral antigens, human papilloma virus (HPV) E6 and E7 antigens, one or more bacterial, viral, or fungal vaginal infections, one or more sexually transmitted diseases, genitourinary cancers, or combinations and modifications thereof.

20. The method of claim 16, wherein the composition enhances proliferation of one or more T cells towards a Th22-type response.

21. A vaginal immunostimulatory composition comprising:
one or more vaginal anti-dendritic cell (DC)-specific antibodies or fragments thereof directed towards one or more specific vaginal DC subsets/macrophages loaded or chemically coupled with one or more antigenic peptides, wherein the antibodies or fragments are directed towards one or more antigens selected from the group consisting of E-cadherin⁺, CD207⁺, CD205⁺CD1c⁺, CD14⁻, DC-ASGPR^{+/-}, CD209^{+/-}, Dectin-1^{+/-}, CD86, CD83, CD209^{+/-}, CD1c, CD14⁺, CD163⁺, LOX-1, CD1d⁺, CD1c⁻, CD14⁻, CD103, β7 integrin, CCR4, CXCR3, and any combinations thereof;
one or more additional ligands selected from the group consisting of heat-killed bacteria, lipoglycans, lipopolysaccharide, lipoteichoic acids, peptidoglycans, synthetic lipoproteins, zymosan, yeast cell wall components, or combinations and modifications thereof and one or more optional pharmaceutically acceptable carriers and adjuvants, wherein the composition is effective to produce an immune response, for a prophylaxis, a therapy or any combination thereof in a human or an animal subject, wherein the DC subsets/macrophages are selected from the group consisting of isolated Langerhans cells (LCs) E-cadherin⁺CD207⁺CD205⁺, isolated vaginal CD1c⁺CD14⁻ DC-ASGPR⁺CD209^{+/-}Dectin-1^{+/-} DCs, isolated vaginal CD1c⁺CD14⁺ CD209^{+/-}DC-ASGPR^{+/-} DCs, CD1c⁻CD14⁺CD163⁺CD209⁺DC-ASGPR^{+/-}Dectin-1^{+/-}LOX-1⁺CD1d⁺ macrophages, and isolated vaginal CD1c⁻CD14⁻ DCs.

22. The composition of claim 21, wherein the antigenic peptides comprise human immunodeficiency virus (HIV) antigens and gene products selected from the group consisting of gag, pol, and env genes, the Nef protein, reverse transcriptase, string of HIV peptides (Hipo5), a HIVgag-derived p24-PLA HIV gag p24 (gag), and other HIV components, cytomegaloviral antigens, herpes simplex viral antigens, human papilloma virus (HPV) E6 and E7 antigens, antigens from bacteria and fungi selected from the group consisting of Prevotella bivia, Prevotella melaminogenica, Gardnerella vaginalis, Trichomonas vaginalis, Mycoplasma hominis, Mobiluncus species, Neisseria gonorrhoeae, Chlamydia trachomatis, Ureaplasma urealyticus, Candida species, Streptococcus species, and Enterobacteriaceae, and cancer peptides are selected from tumor associated antigens comprising antigens from genitourinary tumors such cervix, uterus, ovarian cancer, vaginal cancer, head and neck cancers caused by HPV infections, or combinations and modifications thereof.

23. The composition of claim 21, wherein the composition enhances proliferation of one or more T cells towards a Th22-type response.

24. A method of providing vaginal immunostimulation by activation of one or more vaginal dendritic cell (DC) subsets/macrophages in a human subject for a prophylaxis, a therapy, amelioration of symptoms or any combinations thereof against one or more bacterial, viral, or fungal vaginal infections, one or more sexually transmitted diseases, genitourinary cancers, or any combinations thereof comprising the steps of:
identifying the human subject in need of vaginal immunostimulation for the prophylaxis, the therapy, or a combination thereof against the one or more bacterial, viral, or fungal vaginal infections, one or more sexually transmitted diseases, genitourinary cancers, or any combinations thereof;
isolating one or more vaginal DC subsets/macrophages from the human subject;
exposing the isolated vaginal DC subsets/macrophages to activating amounts of an immunostimulatory composition or a vaccine comprising:
one or more vaginal anti-dendritic cell (DC)-specific antibodies or fragments thereof directed towards one or more specific vaginal DC subsets/macrophages, wherein the antibodies or fragments are directed towards one or more antigens selected from the group consisting of E-cadherin⁺, CD207⁺, CD205⁺ CD1c⁺, CD14⁻, DC-ASGPR^{+/-, CD}209^{+/-}, Dectin-1^{+/-}, CD86, CD83, CD209^{+/-}, CD1c⁻, CD14⁺, CD163⁺, LOX-1, CD1d⁺, CD1c⁻, CD14⁻, CD103, β7 integrin, CCR4, CXCR3, and any combinations thereof;
one or more antigenic peptides loaded or chemically coupled with the DC-specific antibodies or fragments thereof;
one or more ligands selected from the group consisting of heat-killed bacteria, lipoglycans, lipopolysaccharide, lipoteichoic acids, peptidoglycans, synthetic lipoproteins, zymosan, yeast cell wall components, or combinations and modifications thereof; and
a pharmaceutically acceptable carrier to form an activated complex; and
reintroducing the activated DC complex into the human subject.

25. The method of claim 24, further comprising the optional step of measuring a level of one or more agents selected from the group consisting of IFN-γ, TNF-α, IL-5, IL-17, IL-22, and IL-13, wherein a change in the level of the one or more agents is indicative of immunostimulation.

26. The method of claim 24, wherein the ligand is zymosan.

27. The method of claim 24, wherein the DC-specific antibody is humanized

28. The method of claim 24, wherein the reintroduction of the activated DC complex is done intravaginally.

29. The method of claim 24, wherein the antigenic peptides comprise antigens produced by organisms selected from the group consisting of Prevotella bivia, Prevotella melaminogenica, Gardnerella vaginalis, Trichomonas vaginalis, Mycoplasma hominis, Mobiluncus species, Neisseria gonorrhoeae, Chlamydia trachomatis, Ureaplasma urealyticus, Candida species, Treponema pallidum, Streptococcus species, and Enterobacteriaceae.

30. The method of claim 24, wherein the antigenic peptides comprise human immunodeficiency virus (HIV) antigens and gene products selected from the group consisting of gag, pol, and env genes, the Nef protein, reverse transcriptase, string of HIV peptides (Hipo5), a HIVgag-derived p24-PLA HIV gag p24 (gag), and other HIV components, cytomegaloviral antigens, herpes simplex viral antigens, human papilloma virus (HPV) E6 and E7 antigens, or combinations and modifications thereof.

31. The method of claim 24, wherein the antigenic peptides are cancer peptides are selected from tumor associated antigens comprising antigens from genitourinary tumors such cervix, uterus, ovarian cancer, vaginal cancer, head and neck cancers caused by HPV infections, or combinations and modifications thereof.

32. A method of performing a clinical trial to evaluate a candidate drug believed to be useful in treating vaginal diseases, the method comprising:
a) isolating at least one subset of antigen presenting cells (APCs), wherein the APCs possess a distinct phenotype, wherein the subset is selected from at least one of Langerhans cells (LCs) E-cadherin⁺CD207⁺CD205⁺, vaginal CD1c⁺CD14⁻DC-ASGPR⁺CD209^{+/-}Dectin-1^{+/-} dendritic cells (DCs), vaginal CD1c⁺CD14⁺CD209^{+/-} DC-ASGPR^{+/-} DCs, CD1c⁻CD14⁺CD163⁺CD209⁺ DC-ASGPR^{+/-}Dectin-1^{+/-}LOX-1⁺CD1d⁺ macrophages, or vaginal CD1c⁻CD14⁻ DCs,
b) determining the T cell activating activity of the antigen presenting cells isolated from the patient;
c) administering a candidate drug to a first subset of the patients, and
a placebo to a second subset of the patients;
a comparator drug to a second subset of the patients; or
a drug combination of the candidate drug and another active agent to a second subset of patients;
d) repeating step a) after the administration of the candidate drug or the placebo, the comparator drug or the drug combination; and
e) monitoring the T cell activating activity of the antigen presenting cells, wherein a statistically significant change in T cell activating activity indicates that the candidate drug is useful for treating the vaginal disease.
